(19) United States
(12) Patent Application Publication
(10) Pub. No.: US 2012/0020876 A1
(43) Pub. Date: Jan. 26, 2012

(54) HEDGEHOG PATHWAY INHIBITORS

(76) Inventors: Kenneth Paul Olive, New York, NY (US); David Tuveson, Cambridge (GB)

(21) Appl. No.: 13/144,992

(22) PCT Filed: Jan. 22, 2010
(86) PCT No.: PCT/US2010/021816

§ 371 Date: Oct. 6, 2011

Related U.S. Application Data
(60) Provisional application No. 61/205,837, filed on Jan. 23, 2009.

(51) Int. Cl. A61K 51/00; A61K 31/7068; A61K 31/704; A61K 49/10; G01N 33/566; C12N 5/071; A61K 49/04; C12N 5/09; C12N 5/079; A61K 49/22; A61K 31/4418; A61K 31/496; A61K 31/395; A61P 35/00; A61P 35/04; A61P 9/00; A61K 31/4355

(52) U.S. Cl. 424/1.11; 514/278; 514/49; 514/34; 424/9.364; 435/7.21; 435/375; 424/9.4; 424/9.5; 514/357; 514/254.01; 514/183

(57) ABSTRACT

This disclosure generally relates to methods useful for improving, for example, blood vessel density and/or blood vessel patency to a tissue by administration of a hedgehog pathway inhibitor. In certain embodiments, the hedgehog pathway inhibitor is administered with an agent to improve the delivery of the agent to the tissue. In certain embodiments, the tissue comprises tumor tissue.

### TECHNICAL FIELD

This disclosure generally relates to methods useful for improving delivery of agents, e.g., therapeutic and imaging agents, to tissues and more particularly to methods for treating cancerous and non-cancerous tissues, imaging tissues, increasing blood vessel density and patency, and improving drug delivery to tissues, e.g., poorly permeable tissues.

### BACKGROUND

Poor tissue vascularization can hinder blood flow as well as the delivery of both endogenous and exogenous agents, e.g., small molecule and macromolecular compounds to certain tissues. Proangiogenic therapies have been explored to improve tissue vascularization with limited success. Blood flow and drug delivery in poorly vascularized tissues can be improved, in certain instances, by increasing blood vessel patency and/or blood vessel density. Agents having such activity can be used, for example, to diagnose and/or treat cancer by improving delivery of agents (e.g., diagnostic or therapeutic agents) to tumors. Agents having such activity can also be used, for example, to treat occlusive vascular diseases by improving the delivery of blood to an ischemic tissue and/or improving drug delivery to poorly permeable tissues. Accordingly, there exists a need for compositions and methods useful in enhancing tissue vascularization and/or improving delivery of therapeutic and imaging agents.

### SUMMARY

Provided herein are methods useful for improving blood vessel density, blood vessel patency, drug delivery and/or radiation penetration to a tissue. The methods described herein comprise administering a hedgehog pathway inhibitor to a tissue, for example, an ischemic tissue, tumor tissue, non-tumor tissue, and/or poorly permeable tissue. In certain embodiments, administering a hedgehog pathway inhibitor to a tissue increases blood vessel patency and/or blood vessel density in the tissue, thereby enhancing blood flow to the tissue, and/or improving endogenous and/or exogenous agent permeability to the tissue. In certain embodiments, a hedgehog pathway inhibitor is administered with an agent, e.g., a therapeutic and/or imaging agent, to improve the delivery of the agent to the tissue.

In one embodiment, provided is a method of increasing delivery of an agent to a tissue comprising administering a hedgehog pathway inhibitor and the agent to the tissue. In certain embodiments, the hedgehog pathway inhibitor and the agent are administered concurrently. In certain embodiments the hedgehog pathway inhibitor and the agent are administered sequentially.

In certain embodiments, the agent is a therapeutic agent or an imaging agent. In certain embodiments, the imaging agent is a magnetic resonance imaging (MRI) contrast agent, computerized axial tomography (CAT) contrast agent, or positron emission tomography (PET) contrast agent. In certain embodiments, the therapeutic agent is a chemotherapeutic agent.

In cetain embodiments, the tissue comprises autochthonous tissue, stromal tissue, ischemic tissue, or tumor tissue. In certain embodiments the tumor tissue exhibits Hedgehog pathway activation. In certain embodiments, the Hedgehog pathway activation is characterized by one or more of phenotypes selected from group consisting of a Patched (Ptc) loss-of-function phenotype or a Smoothened (Smo) gain-of-function phenotype.

Another embodiment relates to a method of treating a tumor in a mammal, comprising administering to the mammal a therapeutically effective amount of a hedgehog pathway inhibitor and a therapeutically effective amount of a chemotherapeutic agent. In certain embodiments the hedgehog pathway inhibitor and the chemotherapeutic agent are administered concurrently or sequentially. In certain embodiments, the tumor is an autochthonous tumor. In certain embodiments, the autochthonous tumor is a pancreatic tumor, a prostate tumor, a breast tumor, a desmoplastic small round cell tumor, a colon tumor, an ovarion tumor, a bladder tumor, or an osteocarcinoma.

In certain embodiments, the administering comprises administering the hedgehog pathway inhibitor prior to initiating administration of the chemotherapeutic agent. In certain embodiments, the administering comprises administering the hedgehog pathway inhibitor from about 3 days to about 21 days. In certain embodiments, the administering comprises administering the hedgehog pathway inhibitor from about 3 days to about 21 days prior to initiating administration of the chemotherapeutic agent. In certain embodiments, the administering comprises administering the hedgehog pathway inhibitor from about 3 days to about 14 days prior to initiating administration of the chemotherapeutic agent. In certain embodiments, the tumor exhibits Hedgeghog pathway activation. In certain embodiments, the Hedgehog pathway activation is characterized by one or more phenotypes selected from group consisting of a Patched (Ptc) loss-of-function phenotype or a Smoothened (Smo) gain-of-function phenotype.

Certain embodiments relate to any one of the aforementioned methods, where the chemotherapeutic agent is selected from the group consisting of gemcitabine, capecitabine, 5-fluorouracil, floxuridine, doxifluridine, ratitrexed, methotrexate, trimetrexate, thapsigargin, taxol, paclitaxel, docetaxel, actinomycin D, dactinomycin, mercaptopurine, thioguanine, lovastatin, cytosine arabinoside, fludarabine, hydroxyurea, cytarabine, cytarabine, teniposide, topotecan, 9-aminocamptothecin, camptoirinotecan, crisnatol, busulfan, mytomycin C, treosulfan, staurosporine, 1-methyl-4-phenylpyridinium, mercaptopurine, thioguanine, cyclophosphamide, ifosfamide, EB 1089, CB 1093, KH 1060, carmustine, lomustine, mycophenolic acid, tiazofurin, ribavirin, EICAR, cisplatin, carboplatin, oxaliplatin, bevacizumab, mitomycin, dacarbazine, procarbizine, etoposides, prednisolone, trofosfamide, chlorambucil, melphalan, estramustine, dexamethasone, cytarbine, campathecins, bleomycin, doxorubicin, idarubicin, daunorubicin, doxorubicin, epirubicin, pirarubicin, zorubicin, verapamil, mitoxantrone, temozolomide, dactinomycin, plicamycin, bleomycin A2, bleomycin B2, peplomycin, asparaginase, vinblastine, vincristine, vindesine, vinorelbine, imatinib, thalidomide, leucovirin, deferoxamine, lenalidomide, bortezomib, erlotinib, gefitinib, sorafenib, erbitux, and sutinib.

In certain embodiments, provided is a method of increasing blood vessel density in a tissue comprising administering a hedgehog pathway inhibitor to the tissue. In certain embodiments, the administering occurs in vivo.

In certain embodiments, the tissue comprises ischemic tissue, cardiac tissue, brain tissue, comprises stromal tissue, or comprises tumor tissue. In certain embodiments, the tumor tissue exhibits Hedgehog pathway activation. In certain embodiments, the Hedghog pathway activation is characterized by one or more of phenotypes selected from the group consisting of a Patched (Ptc) loss-of-function phenotype or a Smoothened (Smo) gain-of-function phenotype.

In another embodiment, provided is a method of imaging a tissue comprising the steps of administering a hedgehog pathway inhibitor and an imaging agent to the tissue and using an imaging technique to image the tissue. In certain embodiments, the administering comprises administering the hedgehog pathway inhibitor prior to initiating administration of the imaging agent. In certain embodiments, the tissue is cardiac, brain tissue, or tumor tissue. In certain embodiments the tumor tissue exhibits Hedgehog pathway activation. In certain embodiments, the Hedgehog pathway activation is characterized by one or more of phenotypes selected from group consisting of a Patched (Ptc) loss-of-function phenotype or a Smoothened (Smo) gain-of-function phenotype. In certain embodiments, the administering occurs in vivo.

In certain embodiments, the imaging technique is ultrasound, X-ray, MRI, CAT, or PET. In certain embodiments, the imaging agent is an MRI contrast agent, a CAT contrast agent, or a PET contrast agent.

In another embodiment, provided is a method of reducing stromal content in a tissue comprising administering a hedgehog pathway inhibitor to the tissue. In certain embodiments, the tissue comprises ischemic tissue, an autochthonous tissue, or tumor tissue. In certain embodiments, the tumor tissue exhibits Hedgehog pathway activation. In certain embodiments, the Hedgehog pathway activation is characterized by one or more of phenotypes selected from group consisting of a Patched (Ptc) loss-of-function phenotype or a Smoothened (Smo) gain-of-function phenotype.

In another embodiment, provided is a method of increasing blood vessel patency in a tissue comprising administering a hedgehog pathway inhibitor to the tissue. In certain embodiments, the administering occurs in vivo. In certain embodiments, the tissue comprises ischemic tissue, cardiac tissue, brain tissue, tumor tissue. In certain embodiments, the tumor tissue exhibits Hedgehog pathway activation. In certain embodiments, the Hedgehog pathway activation is characterized by one or more of phenotypes selected from the group consisting of a Patched (Ptc) loss-of-function phenotype or a Smoothened (Smo) gain-of-function phenotype.

In another embodiment, provided is a method of promoting angiogenesis in a tissue comprising administering a hedgehog pathway inhibitor to the tissue. In certain embodiments, the administering occurs in vivo. In certain embodiments, the tissue comprises ischemic tissue, cardiac tissue, brain tissue, or tumor tissue. In certain embodiments, the tumor tissue exhibits Hedgehog pathway activation. In certain embodiments, the Hedgehog pathway activation is characterized by one or more of phenotypes selected from the group consisting of a Patched (Ptc) loss-of-function phenotype or a Smoothened (Smo) gain-of-function phenotype.

In another embodiment, provided is a method of imaging a tissue comprising the steps of administering a hedgehog pathway inhibitor to the tissue and using an imaging technique to image the tissue. In certain embodiments, the tissue comprises tumor tissue. In certain embodiments, the tumor tissue exhibits Hedgehog pathway activation. In certain embodiments, the Hedgehog pathway activation is characterized by one or more of phenotypes selected from group consisting of a Patched (Ptc) loss-of-function phenotype or a Smoothened (Smo) gain-of-function phenotype. In certain embodiments, administrating occurs in a mammal. In certain embodiments, the imaging technique is ultrasound or X-ray. In certain embodiments, the tissue is cardiac or brain tissue.

In another embodiment, provided is a method of treating or preventing tumor metastasis, comprising administering to a mammal in need thereof a hedgehog pathway inhibitor and a chemotherapeutic agent. In certain embodiments, the hedgehog pathway inhibitor and the chemotherapeutic agent are administered concurrently or sequentially.

In certain embodiments, the tumor is a pancreatic tumor, a prostate tumor, a breast tumor, a desmoplastic small round cell tumor, a colon tumor, an ovarion tumor, a bladder tumor, or an osteocarcinoma.

In certain embodiments, the chemotherapeutic agent is selected from the group consisting of gemcitabine, capecitabine, 5-fluorouracil, floxuridine, doxifluridine, ratitrexed, methotrexate, trimetrexate, thapsigargin, taxol, paclitaxel, docetaxel, actinomycin D, dactinomycin, mercaptopurine, thioguanine, lovastatin, cytosine arabinoside, fludarabine, hydroxyurea, cytarabine, cytarabine, teniposide, topotecan, 9-aminocamptothecin, camptoirinotecan, crisnatol, busulfan, mytomycin C, treosulfan, staurosporine, 1-methyl-4-phenylpyridinium, mercaptopurine, thioguanine, cyclophosphamide, ifosfamide, EB 1089, CB 1093, KH 1060, carmustine, lomustine, mycophenolic acid, tiazofurin, ribavirin, EICAR, cisplatin, carboplatin, oxaliplatin, bevacizumab, mitomycin, dacarbazine, procarbizine, etoposides, prednisolone, trofosfamide, chlorambucil, melphalan, estramustine, dexamethasone, cytarbine, campathecins, bleomycin, doxorubicin, idarubicin, daunorubicin, doxorubicin, epirubicin, pirarubicin, zorubicin, verapamil, mitoxantrone, temozolomide, dactinomycin, plicamycin, bleomycin A2, bleomycin B2, peplomycin, asparaginase, vinblastine, vincristine, vindesine, vinorelbine, imatinib, thalidomide, leucovirin, deferoxamine, lenalidomide, bortezomib, erlotinib, gefitinib, sorafenib, erbitux, and sutinib.

Certain embodiments relate to any of the aforementioned methods, where the hedgehog pathway inhibitor is selected from the group consisting of a compound of Formula I, Formula II, or Formula III:

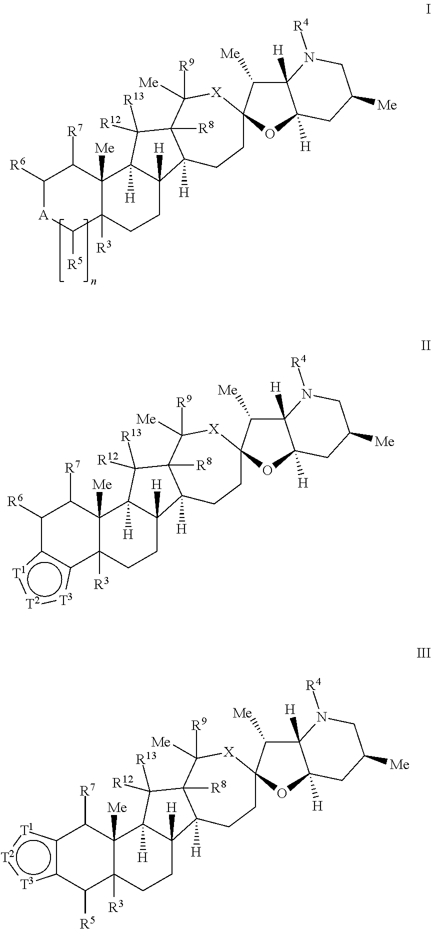

or a pharmaceutically acceptable salt thereof;

wherein A is:

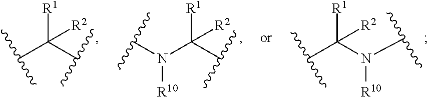

n is 0 or 1;

X is a bond or —CH₂—;

R¹ is selected from —H, optionally substituted alkyl, optionally substituted alkenyl, optionally substituted alkynyl, optionally substituted aryl, optionally substituted aralkyl, optionally substituted cycloalkyl, nitrile, optionally substituted heterocycloalkyl, —OR¹⁰, —N(R¹⁰)(R¹⁰), —NR¹⁰SO₂R¹⁰, —N(R¹⁰)CO₂R¹⁰, —N(R¹⁰)C(O)R¹⁰, —OC(O)R¹⁰, and a sugar;

R² is selected from —H, optionally substituted alkyl, optionally substituted alkenyl, optionally substituted alkynyl, optionally substituted aryl, optionally substituted aralkyl, optionally substituted cycloalkyl, nitrile, and optionally substituted heterocycloalkyl; or R¹ and R² taken together form ═O, ═S, ═N(OR), ═N(R)—, ═N(NR₂), ═C(R)₂;

R³ and R⁵, are, independently, selected from —H, optionally substituted alkyl, optionally substituted aralkyl, optionally substituted alkenyl, and optionally substituted alkynyl; or R³ and R⁵ taken together form a bond;

R⁶ and R⁷ are, independently, selected from —H, optionally substituted alkyl, optionally substituted aralkyl, optionally substituted alkenyl, and optionally substituted alkynyl; or R⁶ and R⁷ taken together form a bond;

R⁸ and R⁹ taken together form a bond;

R⁴ is selected from —H, optionally substituted alkyl, optionally substituted alkenyl, optionally substituted alkynyl, optionally substituted aryl, optionally substituted aralkyl, optionally substituted cycloalkyl, optionally substituted heterocycloalkyl, optionally substituted aralkyl, optionally substituted heteroaryl, optionally substituted heteroaralkyl, optionally substituted haloalkyl, —OR¹⁰, —C(O)R¹⁰, —CO₂R¹⁰, —SO₂R¹⁰, —C(O)N(R¹⁰)(R¹⁰), —[C(R)₂]_{q}—R¹⁰, —[(W)—N(R¹⁰)C(O)]_{q}R¹⁰, —[(W)—C(O)]_{q}R¹⁰, —[(W)—C(O)O]_{q}R¹⁰, —[(W)—OC(O)]_{q}R¹⁰, —[(W)—SO₂]_{q}R¹⁰, —[(W)—N(R¹⁰)SO₂]_{q}R¹⁰, —[(W)—C(O)N(R¹⁰)]_{q}R¹⁰, —[(W)—O]_{q}R¹⁰, —[(W)—N(R)]_{q}R¹⁰, and —[(W)—S]_{q}R¹⁰;

each q, independently, for each occurrence, is 1, 2, 3, 4, 5, or 6;

each R¹⁰ is, independently, selected from —H, optionally substituted alkyl, optionally substituted alkenyl, optionally substituted aralkyl, optionally substituted alkynyl, optionally substituted aryl, optionally substituted cycloalkyl, optionally substituted heterocycloalkyl, optionally substituted aralkyl, optionally substituted heteroaryl, optionally substituted heteroaralkyl and —[C(R)₂]ₚ—R¹¹; wherein p is 0-6; or any two occurrences of R¹⁰ on the same substituent can be taken together to form a 4-8 membered optionally substituted ring which contains 0-3 heteroatoms selected from nitrogen, oxygen, sulfur, and phosphorus;

each R¹¹ is, independently, selected from hydroxyl, —N(R)COR, —N(R)C(O)OR, —N(R)SO₂(R), —C(O)N(R)₂, —OC(O)N(R)(R), —SO₂N(R)(R), —N(R)(R), —COOR, —C(O)N(OH)(R), —OS(O)₂OR, —S(O)₂OR, —S(O)₂R, —OP(O)(OR)(OR), —NP(O)(OR)(OR), and —P(O)(OR)(OR);

each R is, independently, selected from —H, optionally substituted alkyl, optionally substituted alkenyl, optionally substituted alkynyl, optionally substituted aryl, optionally substituted cycloalkyl and optionally substituted aralkyl;

R¹² and R¹³ are, independently, selected from —H, optionally substituted alkyl, optionally substituted alkenyl, optionally substituted alkynyl, optionally substituted aryl, optionally substituted aralkyl, optionally substituted cycloalkyl, nitrile, optionally substituted heterocycloalkyl, —OR¹⁰, —N(R¹⁰)(R¹⁰), —NR¹⁰SO₂R¹⁰, —N(R¹⁰)CO₂R¹⁰, —N(R¹⁰)C(O)R¹⁰, and —OC(O)R¹⁰; or R¹² and R¹³ taken together form ═O, ═S, ═N(OR), ═N(R)—, ═N(NR₂), ═C(R)₂;

each W is, independently for each occurrence, selected from an optionally substituted alkyl diradical, optionally substituted alkenyl diradical, optionally substituted alkynyl diradical, optionally substituted aryl diradical, optionally substituted cycloalkyl diradical, optionally substituted heterocycloalkyl diradical, optionally substituted aralkyl diradical, optionally substituted heteroaryl diradical and an optionally substituted heteroaralkyl diradical;

and

T¹-T²-T³ is selected from Y-B-A, B-Y-A, and A-B-Y; wherein each of A and B is, independently, selected from nitrogen, sulfur and —C(R¹⁴)₂— and Y is selected from —O—, —S—, and —N(R¹⁵)—;

wherein R¹⁴ is, independently, selected from —H, optionally substituted alkyl, optionally substituted alkenyl, optionally substituted alkynyl, optionally substituted cycloalkyl, optionally substituted aryl, optionally substituted aralkyl, optionally substituted heteroaryl, optionally substituted heteroaralkyl, perhaloalkyl, halo, nitro, nitrile, ═O, —SR¹⁰, —OR¹⁰, —N(R¹⁰)(R¹⁰), —C(O)R¹⁰, —CO₂R¹⁰, —OC(O)R¹⁰, —C(O)N(R¹⁰)(R¹⁰), —N(R¹⁰)C(O)R¹⁰, —N(R¹⁰)C(O)N(R¹⁰)(R¹⁰), —S(O)R¹⁰, —S(O)₂R¹⁰, —S(O)₂N(R¹⁰)(R¹⁰), —N(R¹⁰)S(O)₂R¹⁰ and —[C(R¹⁰)₂]q-R¹¹; and wherein R¹⁵ is selected from —H, optionally substituted alkyl, optionally substituted alkenyl, optionally substituted alkynyl, optionally substituted cycloalkyl, optionally substituted aryl, optionally substituted aralkyl, optionally substituted heteroaryl, optionally substituted heteroaralkyl, perhaloalkyl, —C(O)R¹⁰, —CO₂R¹⁰, —C(O)N(R¹⁰)(R¹⁰), S(O)R¹⁰, —S(O)₂R¹⁰, —S(O)₂N(R¹⁰)(R¹⁰), and —[C(R)₂]_{q}—R¹¹.

The details of one or more embodiments of the methods described herein are set forth in the accompanying drawings and the description below. Other features, objects, and advantages of the disclosure will be apparent from the description and drawings, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1F. Mice bearing transplanted pancreatic tumors or KPC mice with in-situ tumors were treated Q3Dx4 with control saline or gemcitabine. Asterisks indicate P<0.05, Mann-Whitney U. FIG. 1A: Box plots indicate % change in volume over 12 days in saline-(blue) or gemcitabine-(red) treated tumors from several transplantation models. FIG. 1B: Immunohistochemistry for phospho-histone H3 was quantified, revealing significantly lower proliferative rates in gemcitabine treated transplanted tumors. Positive control: small intestines. FIG. 1C: Immunohistochemistry for cleaved caspase 3 was quantified, showing no significant changes in apoptosis in gemcitabine treated transplanted tumors. Positive control: small intestines. FIG. 1D: Percentage volume change of KPC tumors treated for 12 days with 0, 50 or 100 mg/kg gemcitabine. Two responding tumors are highlighted in yellow. Solid lines: mean volume change; dashed lines: means without responders. FIG. 1E: Proliferation (measured as above) was significantly diminished in KPC tumors treated with 100 mg/kg gemcitabine (P=0.003, Mann-Whitney U). Solid lines=mean; dashed lines=mean without responders. FIG. 1F: Apoptosis was significantly elevated in two responding KPC tumors (yellow circles) but unchanged in most KPC tumors treated with gemcitabine. Solid lines=mean; dashed lines=mean without responders.

FIGS. 2A-2H. FIGS. 2A and 2B: Infused Lycospersicon esculentum lectin (red) detected patent blood vessels and CD31 immunofluorescence (green) denoted total vascular content. Scale bars=100 μm. Widespread co-labeling of lectin and CD31 indicated a patent vasculature (arrows) in transplanted tumors (FIG. 2A, N=5) while only a minority of CD31⁺ vessels (dashed arrows) were perfused with lectin (solid arrows) in KPC tumors (FIG. 2B, N=3). FIGS. 2C and 2D: Lycospersicon esculentum lectin (red) and doxorubicin (green) were infused prior to euthanasia and visualized by direct immunofluorescence. Scale bar=200 μm. Doxorubicin was more effectively delivered to transplanted tumors (FIG. 2C, N=5), than to KPC tumors, relative to surrounding tissues (FIG. 2D, N=4). FIGS. 2E and 2F: Contrast ultrasonography using microbubbles (green) visualized the rapid perfusion of transplanted tumors (FIG. 2E, N=6) in contrast to the poor perfusion observed in KPC tumors (FIG. 2F, N=8). Tumors outlined in yellow. Scale bars=1 mm. FIGS. 2G and 2H: DCE-MRI demonstrated increased perfusion and extravasation of Gd-DTPA (high delivery=yellow/white) in transplanted tumors (FIG. 2G, N=6) compared to KPC tumors (FIG. 2H, N=6). Tumors outlined in blue. Scale bars=2 mm.

FIGS. 3A-3I. CD31 immunohistochemistry was performed on transplanted (FIGS. 3A and 3B), KPC (FIGS. 3C and 3D) and human (FIGS. 3E and 3F) pancreatic tumors and photographed at low power (FIGS. 3A, 3C, 3E; scale bar=50 μm) or high power (FIGS. 3B, 3D, 3F; scale bar=20 μm). Arrows denote blood vessels. FIG. 3A: Peripheral regions of transplanted tumors (T) were densely vascularized compared to surrounding tissues (S) and more central regions (C). FIG. 3B: Blood vessels are directly juxtaposed to tumors cells in transplanted tumors. FIG. 3C: Fewer blood vessels are apparent in the parenchyma of KPC tumors (T) despite extensive vascularization of surrounding capsular tissues (S). FIG. 3D: Neoplastic cells in KPC tumors are separated from blood vessels by the stroma. FIGS. 3E and 3F: Similarly, human pancreatic tumors (T) are poorly vascularized despite ample vascularization of surrounding tissues (S). FIG. 3G: Mean Vessel Density (MVD) was measured in KPC tumors (KPC), syngeneic autografts (Syn), orthotopic xenografts (Ortho), normal murine pancreas (Norm), adjacent surrounding tissues in KPC tumors (Adj), human normal pancreatic tissues and human pancreatic tumor tissues. KPC and human pancreatic tumors had lower mean vessel densities compared to transplanted tumors and normal tissues (P<0.004 for all four respective comparisons, Mann-Whitney U). FIG. 3H: MVD is significantly lower in the central regions of human PDAs compared to peripheral (P) and central (C) regions of normal human pancreas or chronic pancreatitis samples (**P<0.0015, ***P<0.0001, Mann-Whitney U). FIG. 3I: The distance separating blood vessels and neoplastic cells was significantly higher in KPC tumors (KPC) and human PDA (Human) than in syngeneic autografts (Syn) or orthotopic xenografts (Ortho).

FIGS. 4A-4I. Mice received one of five regimens: not treated (NT), vehicles (V), gemcitabine (G), Compound A (I), or Compound A and gemcitabine (IG). FIG. 4A: The concentration of Compound A in tumor tissues is shown for mice treated with a single dose (SD), daily for 4 days (Early) or at the end of a survival study (Endpoint) as well as in kidneys from mice treated at endpoint. FIG. 4B: Gli1 expression (measured by RTPCR) was significantly lower in Compound A and Compound A/gem treated KPC tumors than control KPC mice treated for 4 days (P<0.05). FIG. 4C: MVD was significantly elevated in Compound A and Compound A/gem treated KPC tumors after 8-12 days (P<0.05). FIG. 4D: Doxorubicin fluorescence per unit area was significantly elevated in Compound A/gem treated tumors after 8-12 days (P=0.03, Mann-Whitney U). FIG. 4E: Following treatment with the indicated regimens, all mice were administered a single dose of gemcitabine and the concentration of fluorine-bearing metabolites was determined by extracted samples by ¹⁹F NMR. The concentration of gemcitabine metabolites in KPC tumor tissues was significantly elevated in Compound A/gem treated tumors following 10 days of treatment. Plot indicates total concentration of fluorine-bearing gemcitabine metabolites detected by ¹⁹F NMR, in relative units (P<0.04, Mann-Whitney U). FIG. 4F: Proliferation of KPC tumors (determined as in FIG. 1E) was decreased in gemcitabine and Compound A/gem treated tumors after 4 days (early) or 8-12 days (intermediate) but unchanged in Compound A treated tumors. FIG. 4G: Apoptosis was elevated in a subset of Compound A/gem treated KPC tumors after 8-12 days (P=0.17), but unchanged in control cohorts. FIG. 4H: Survival of KPC mice following the detection of 5-10 mm pancreatic tumors was significantly extended in Compound A/gem treated mice compared to controls (P=0.001 Log-Rank Test, Hazard Ratio=6.36). FIG. 4I: Compound A/gem treated mice also had significantly fewer liver metastases compared to control-treated cohorts (P=0.015, Fisher's Exact).

FIGS. 5A-5D. FIG. 5A: HPLC confirms the short half-life of gemcitabine (dFdC) in the blood of normal mice. FIG. 5B: HPLC Results of FIG. 5A correlate with the accumulation of the inactive metabolite difluorodeoxyuridine (dFdU) as depicted in FIG. 5B. FIG. 5C: Quantitative RT-PCR was performed on RNA from tumor tissues for genes implicated in the cellular response to gemcitabine. P-values for Mann-Whitney U tests are indicated below each gene, showing significant differences only in dCK and RRM2. FIG. 5D: These differences were less apparent in cohorts of gemcitabine-treated tumors.

FIGS. 6A-6F. FIG. 6A: Perfusion and immunofluorescence for CD31 and lectin was performed as described in FIG. 2A. The percent of CD31⁺ blood vessels that were labeled with lectin was determined in normal pancreas (Norm) as well as KPC and transplanted tumors. KPC tumors had significantly fewer patent vessels than transplanted tumors and normal tissues (*P<0.05, Mann-Whitney U). FIGS. 6B-6F: Lectin and doxorubicin were perfused as in FIG. 2C. Normal tissues (FIG. 6B), subcutaneous transplanted tumors (FIG. 6C) and orthotopic transplanted tumors (FIG. 6D) exhibit ample vascular labeling (red, arrows) and doxorubicin content (green), with DAPI (blue) denoting nuclear content. In panel B, A=acinar, I=Islets, D=ducts, and left inset panel shows only the doxorubicin channel, demonstrating doxorubicin uptake in normal ductal cells. FIG. 6E: Pancreata from KPC mice demonstrate lectin labeling (arrows) and doxorubicin content in adjacent PanIN tissue. FIG. 6F: In the invasive area of the PDA tumor (T), doxorubicin perfusion is apparent immediately adjacent to the few lectin-labeled vessels (arrows). Yellow triangles denote the sharp demarcation between tumor and adjacent acinar pancreatic tissue. Scale bars=100 uM.

FIGS. 7A-7F. Representative images are presented of Masson's trichrome-stained tumors from subcutaneous autografts (FIG. 7A) and orthotopic xenografts (FIG. 7B), as well as gemcitabine-resistant KPC tumors (FIG. 7C) and primary human pancreatic tumors (FIG. 7D). Yellow arrows indicate stromal fibers, when detected. Tumors from the transplantation models generally exhibit little stroma while KPC tumors and human tumors have a prominent stromal component. Of note, the two gemcitabine-sensitive tumors had a lower stromal content (FIG. 7E) and a higher vascular density (FIG. 7F) than other KPC tumors. Black arrows denote blood vessels. Scale bars for all panels are 20 μm.

FIGS. 8A-8F. Representative images from normal human pancreas (FIGS. 8A and 8B) and PDA (FIGS. 8C-8F). Adjacent paraffin sections were stained with hematoxylin and eosin (FIGS. 8A, 8C and 8E) and anti-CD31 (FIGS. 8B, 8D and 8F). Dashed boxes in FIG. 8C and FIG. 8D indicate regions shown at higher magnification in FIG. 8E and FIG. 8F, respectively. Similar to observations in mice, human normal pancreatic tissue contains a dense network of fine capillaries surrounding the acini and ducts (FIG. 8B, arrows), whereas regions of invasive cancer exhibit remarkably few blood vessels (FIGS. 8D and 8F, arrow). Scale bars in panels (FIGS. 8A, 8B, 8E and 8F)=50 μm; bars in panels (FIGS. 8C and 8D)=200 μm.

FIGS. 9A-9L. KPC tumors were treated for 8-12 days with vehicle (FIGS. 9A, 9E and 9I), gemcitabine (FIGS. 9B, 9F and 9J), Compound A (FIGS. 9C, 9G and 9J) or Compound A/gem (FIGS. 9D, 9H and 9L). FIGS. 9A-9D H&E stained sections demonstrate the loss of cellular and acellular stroma following treatment with Compound A and Compound A/gem, resulting in densely packed tumor cells. Those treated with Compound A/gem contained regions of severe nuclear and cellular atypia (arrows). FIGS. 9E-9H: CD31 immunohistochemistry demonstrates increased MVD following Compound A and Compound A/gem treatment. FIGS. 9I-9L: Doxorubicin immunofluorescence (green) demonstrates increased content in Compound A treated tumors. Blue=DAPI. A heterogeneous pattern of doxorubicin staining was noted in Compound A and Compound A/gem treated tumors. Scale bars=100 μm.

FIGS. 10A-10D. Tumors in mice treated with saline (FIG. 10A), gemcitabine 100 mg/kg twice weekly (FIG. 10B), Compound A 40 ug/kg/day (FIG. 10C) and Compound A/gem (FIG. 10D) were monitored by 3D high resolution ultrasonography. No objective responses were observed in saline treated mice. 2/10 gemcitabine treated mice exhibited an objective response (example in first panel). 2/10 Compound A treated mice exhibited an objective response (example in first panel). Most Compound A/gem treated tumors (8/10) responded at least transiently to treatment, with some showing prolonged stable disease (red tracing, second panel, fourth panel).

FIG. 11. dFdCTP and ATP were detected by HPLC in spleen, normal pancreas and pancreatic tumor tissue from mice on study. Suitability of the tissue was determined by the level of ATP in the sample.

FIG. 12. Partial restoration of vessel patency in Compound A/gem treated pancreatic tumors. Normal mice (Norm), untreated KPC mice (NT) or KPC mice treated with gemcitabine (G), Compound A (I) or Compound A/gem (I/G) for 10 days were perfused with lectin for 15 minutes prior, and then immunohistochemistry for CD31 was performed on isolated pancreas or tumor tumor tissues. The percent of CD31 positive vessels that were perfused with lectin was scored, showing that Compound A and Compound A/gem treated mice had increased vessel patency compared to untreated or gemcitabine tumors.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in, for example, Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Certain compounds of the present invention can comprise one or more asymmetric centers, and thus can exist in various isomeric forms, e.g., stereoisomers and/or diastereomers. Thus, inventive compounds and pharmaceutical compositions thereof may be in the form of an individual enantiomer, diastereomer or geometric isomer, or may be in the form of a mixture of stereoisomers. Enantiomers, diastereomers and geometric isomers may be isolated from racemic mixtures by any method known to those skilled in the art, including chiral high pressure liquid chromatography (HPLC) and the formation and crystallization of chiral salts or prepared by asymmetric syntheses; see, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a substituent at each substitutable position of the group, and when more than one position in any given structure may be substituted with more than one substituent selected from a specified group, the substituent may be either the same or different at every position. Combinations of substituents envisioned by this invention are those that result in the formation of stable compounds. The term “stable”, as used herein, refers to compounds that are not substantially altered when subjected to conditions to allow for their synthesis, manufacture, purification and/or storage.

The term “optionally substituted” refers to any chemical group, such as alkyl, cycloalkyl aryl, and the like, wherein one or more hydrogen may be replaced with another substituent, which includes, but is not limited to, halo, azide, alkyl, alkenyl, alkynyl, cycloalkyl, hydroxyl, alkoxyl, amino, nitro, nitrile, sulfhydryl, imino, amido, phosphonate, phosphinate, —CO₂H, —CHO, silyl, alkoxy, alkylthio, sulfonyl, sulfonamido, ester, ═O, ═S, heterocyclyl, aryl, aralkyl, heteroaryl, heteroaralkyl, perfluoroalkyl (e.g., —CF₃) or the like.

When a range of values is listed, it is intended to encompass each value and sub-range within the range. For example, an alkyl group containing 1-6 carbon atoms (C₁₋₆ alkyl) is intended to encompass, C₁, C₂, C₃, C₄, C₅, C₆, C₁₋₆, C₂₋₆, C₃₋₆, C₄₋₆, C₅₋₆, C₁₋₅, C₂₋₅, C₃₋₅, C₄₋₅, C₁₋₄, C₂₋₄, C₃₋₄, C₁₋₃, C₂₋₃, and C₁₋₂ alkyl.

The term “alkyl,” as used herein, refers to saturated, straight- or branched-chain hydrocarbon radical containing between one and thirty carbon atoms. In certain embodiments, the alkyl group contains 1-20 carbon atoms. In certain embodiments, the alkyl group contains 1-10 carbon atoms. In certain embodiments, the alkyl group contains 1-9 carbon atoms. In certain embodiments, the alkyl group contains 1-8 carbon atoms. In certain embodiments, the alkyl group contains 1-7 carbon atoms. In certain embodiments, the alkyl group contains 1-6 carbon atoms. In certain embodiments, the alkyl group contains 1-5 carbon atoms. In certain embodiments, the alkyl group contains 1-4 carbon atoms. In certain embodiments, the alkyl group contains 1-3 carbon atoms. In certain embodiments, the alkyl group contains 1-2 carbon atoms. In certain embodiments, the alkyl group contains 1 carbon atom. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like.

The term “alkenyl,” as used herein, denotes a straight- or branched-chain hydrocarbon radical having at least one carbon-carbon double bond by the removal of a single hydrogen atom, and containing between two and thirty carbon atoms. In certain embodiments, the alkenyl group contains 2-20 carbon atoms. In certain embodiments, the alkenyl group contains 2-10 carbon atoms. In certain embodiments, the alkenyl group contains 2-9 carbon atoms. In certain embodiments, the alkenyl group contains 2-8 carbon atoms. In certain embodiments, the alkenyl group contains 2-7 carbon atoms. In certain embodiments, the alkenyl group contains 2-6 carbon atoms. In certain embodiments, the alkenyl group contains 2-5 carbon atoms. In certain embodiments, the alkenyl group contains 2-4 carbon atoms. In certain embodiment, the alkenyl group contains 2-3 carbon atoms. In certain embodiments, the alkenyl group contains 2 carbon atoms. Alkenyl groups include, for example, ethenyl, propenyl, butenyl, 1-methyl-2-buten-1-yl, and the like.

The term “alkynyl,” as used herein, denotes a straight- or branched-chain hydrocarbon radical having at least one carbon-carbon triple bond by the removal of a single hydrogen atom, and containing between two and thirty carbon atoms. In certain embodiments, the alkynyl group contains 2-20 carbon atoms. In certain embodiments, the alkynyl group contains 2-10 carbon atoms. In certain embodiments, the alkynyl group contains 2-9 carbon atoms. In certain embodiments, the alkynyl group contains 2-8 carbon atoms. In certain embodiments, the alkynyl group contains 2-7 carbon atoms. In certain embodiments, the alkynyl group contains 2-6 carbon atoms. In certain embodiments, the alkynyl group contains 2-5 carbon atoms. In certain embodiments, the alkynyl group contains 2-4 carbon atoms. In certain embodiments, the alkynyl group contains 2-3 carbon atoms. In certain embodiments, the alkynyl group contains 2 carbon atoms. Representative alkynyl groups include, but are not limited to, ethynyl, 2-propynyl (propargyl), 1-propynyl, and the like.

The terms “cycloalkyl”, used alone or as part of a larger moiety, refer to an optionally substituted saturated monocyclic or bicyclic hydrocarbon ring system having from 3-15 carbon ring members. In certain embodiments, cycloalkyl groups contain 3-10 carbon ring members. In certain embodiments, cycloalkyl groups contain 3-9 carbon ring members. In certain embodiments, cycloalkyl groups contain 3-8 carbon ring members. In certain embodiments, cycloalkyl groups contain 3-7 carbon ring members. In certain embodiments, cycloalkyl groups contain 3-6 carbon ring members. In certain embodiments, cycloalkyl groups contain 3-5 carbon ring members. Cycloalkyl groups include, without limitation, cyclopropyl, cyclobutyl, cyclopentyl, cyclohexyl, cycloheptyl, and cyclooctyl. The term “cycloalkyl” also includes saturated hydrocarbon ring systems that are fused to one or more aryl or heteroaryl rings, such as decahydronaphthyl or tetrahydronaphthyl, where the point of attachment is on the saturated hydrocarbon ring.

The term “aryl” used alone or as part of a larger moiety as in “aralkyl”, refers to an optionally substituted aromatic monocyclic and bicyclic hydrocarbon ring system having a total of 6-10 carbon ring members. The term “aryl” may be used interchangeably with the term “aryl ring”. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aryl ring is fused to one or more non-aromatic rings, such as indanyl, phthalimidyl or tetrahydronaphthalyl, and the like, where the point of attachment is on the aryl ring.

The term “aralkyl” refers to an alkyl group substituted by aryl group wherein the point of attachment is on the alkyl group, and wherein the alkyl and aryl groups independently are optionally substituted.

The term “heteroatom” refers to boron, phosphorus, selenium, nitrogen, oxygen, or sulfur, and includes any oxidized form of nitrogen or sulfur, and any quaternized form of a basic nitrogen.

The terms “heteroaryl” used alone or as part of a larger moiety, e.g., “heteroaralkyl”, refer to an optionally substituted aromatic monocyclic or bicyclic hydrocarbon ring system having 5-10 ring atoms wherein the ring atoms comprise, in addition to carbon atoms, from one to five heteroatoms. When used in reference to a ring atom of a heteroaryl group, the term “nitrogen” includes a substituted nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaryl ring is fused to one or more aryl, cycloalkyl or heterocycloalkyl rings, wherein the point of attachment is on the heteroaryl ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl.

The term “heteroaralkyl” refers to an alkyl group substituted by a heteroaryl group wherein the point of attachment is on the alkyl group, and wherein the alkyl and heteroaryl portions independently are optionally substituted.

As used herein, the terms “heterocycloalkyl” or “heterocyclyl” refer to a stable non-aromatic optionally substituted 5-7 membered monocyclic hydrocarbon or stable non-aromatic optionally substituted 7-10 membered bicyclic hydrocarbon that is either saturated or partially unsaturated, and having, in addition to carbon atoms, one or more heteroatoms. When used in reference to a ring atom of a heterocycloalkyl group, the term “nitrogen” includes a substituted nitrogen. The point of attachment of a heterocycloalkyl group may be at any of its heteroatom or carbon ring atoms that results in a stable structure. Examples of heterocycloalkyl groups include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. “Heterocycloalkyl” also include groups in which the heterocycloalkyl ring is fused to one or more aryl, heteroaryl or cycloalkyl rings, such as indolinyl, chromanyl, phenanthridinyl, or tetrahydroquinolinyl, where the radical or point of attachment is on the heterocycloalkyl ring.

The term “unsaturated”, as used herein, means that a moiety has one or more double and/or triple bonds.

As used herein, the term “partially unsaturated” refers to a ring moiety that includes at least one double or triple bond. The term “partially unsaturated” is intended to encompass rings having multiple sites of unsaturation, but is not intended to include aromatic groups, such as aryl or heteroaryl moieties, as defined herein.

The term “diradical” as used herein refers to optionally substituted alkyl, alkenyl, alkynyl, aryl, cycloalkyl, heterocycloalkyl, aralkyl, heteroaryl, and heteroaralkyl groups, wherein 2 hydrogen atoms are removed to form a divalent moiety. Diradicals are typically end with a suffix of “-ene”. For example, alkyl diradicals are referred to as alkylenes (for example:

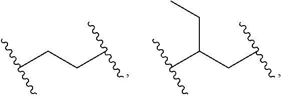

and —(CR′₂)ₓ— wherein R′ is hydrogen or other substituent and x is 1-6, inclusive); alkenyl diradicals are referred to as “alkenylenes”; alkynyl diradicals are referred to as “alkynylenes”; aryl and aralkyl diradicals are referred to as “arylenes” and “aralkylenes”, respectively (for example:

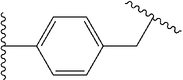

); heteroaryl and heteroaralkyl diradicals are referred to as “heteroarylenes” and “heteroaralkylenes”, respectively (for example:

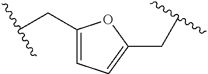

); cycloalkyl diradicals are referred to as “cycloalkylenes”; heterocycloalkyl diradicals are referred to as “heterocycloalkylenes”; and the like.

The terms “halo” and “halogen” as used herein refer to an atom selected from fluorine (fluoro, —F), chlorine (chloro, —Cl), bromine (bromo, —Br), and iodine (iodo, —I).

As used herein, the term “haloalkyl” refers to an alkyl group, as described herein, wherein one or more of the hydrogen atoms of the alkyl group is replaced with one or more halogen atoms. In certain embodiments, the haloalkyl group is a perhaloalkyl group, that is, having all of the hydrogen atoms of the alkyl group replaced with halogens (e.g., such as the perfluoroalkyl group —CF₃).

The term “sugar” as used herein refers to a natural or an unnatural monosaccharide, disaccharide or polysaccharide. The sugar may be covalently bonded to the compound of the present invention through an oxygen, nitrogen or sulfur linkage or through an alkyl linkage. In certain embodiments the saccharide moiety may be covalently bonded to a steroidal alkaloid of the present invention at an anomeric center of a saccharide ring. Exemplary sugars include, but are not limited to, 1,2 and 1,3 hydroxy sugars (e.g., glycerol, erythritol, threitol, ribitol, arabinitol, xylitol, allitol, altritol, galactitol, sorbitol, mannitol and iditol), hexoses (e.g., allose, altrose, glucose, mannose, gulose, idose, galactose and talose), pentoses (e.g., ribose, arabinaose, xylose and lyxose), maltitol, lactitol and isomalt.

As used herein, the term “nitrile” refers to the group —CN.

As used herein, the term “nitro” refers to the group —NO₂.

As used herein, the term “hydroxyl” or “hydroxy” refers to the group —OH.

As used herein, the ther “amino” refers to the group —NR′₂, wherein each R′ is, independently, hydrogen or a carbon moiety, such as, for example, an alkyl, alkenyl, alkynyl, aryl or heteroaryl group.

As used herein, the term “carbonyl” refers to the group —C(═O)R′, wherein R′ is, independently, a carbon moiety, such as, for example, an alkyl, alkenyl, alkynyl, aryl or heteroaryl group.

As used herein, the term “ester” refers to the group —C(═O)OR′ or —OC(═O)R′ wherein each R′ is, independently, a carbon moiety, such as, for example, an alkyl, alkenyl, alkynyl, aryl or heteroaryl group.

As used herein, the term “amide” or “amido” refers to the group —C(═O)N(R′)₂ or —NR′C(═O)R′ wherein each R′ is, independently, hydrogen or a carbon moiety, such as, for example, an alkyl, alkenyl, alkynyl, aryl or heteroaryl group.

As used herein, the term “imide” or “imido” refers to the group —C(═NR′)N(R′)₂ or —NR′C(═NR′)R′ wherein each R′ is, independently, hydrogen or a carbon moiety, such as, for example, an alkyl, alkenyl, alkynyl, aryl or heteroaryl group.

As used herein “ether” refers to the group —OR′ wherein R′ is a carbon moiety, such as, for example, an alkyl, alkenyl, alkynyl, aryl or heteroaryl group.

As used herein “silyl” refers to the group —SiR′ wherein R′ is a carbon moiety, such as, for example, an alkyl, alkenyl, alkynyl, aryl or heteroaryl group.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Compounds useful in the methods described herein, e.g., hedgehog pathway inhibitors, therapeutic agents, and imaging agents, may contain a basic functional group, such as amino or alkylamino, and are thus capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately treating the compound in its free base form with a suitable organic or inorganic acid, and isolating the salt thus formed during subsequent purification. Representative salts include salts derived from suitable inorganic and organic acids, e.g., hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable acid addition salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like (see, for example, Berge et al. (1977) “Pharmaceutical Salts”, J. Pharm. Sci. 66:1-19)

In certain cases, the compounds useful in the methods described herein may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately treating the compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali to or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

As used herein, the term “tautomer” includes two or more interconvertable compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may be catalyzed by acid or base. Exemplary tautomerizations include keto-to-enol; amide-to-imide; lactam-to-lactim; enamine-to-imine; and enamine-to-(a different)-enamine tautomerizations.

“Hedgehog pathway activation” refers to an aberrant modification or mutation of a Hedgehog ligand (aka hedgehog protein), Patched (Ptc) gene or Smoothened (Smo) gene, or a change in the level of expression of a Ptc gene or Smo gene (e.g., a decrease or increase, respectively), which results in a phenotype which resembles contacting a cell with a hedgehog ligand, e.g., aberrant activation of a hedgehog pathway.

“Patched (Ptc) loss-of-function” refers to an aberrant modification or mutation of a Ptc gene or a decrease (or loss) in the level of expression of the Ptc gene, which results in a phenotype which resembles contacting a cell with a hedgehog ligand, e.g., aberrant activation of a hedgehog pathway.

“Smoothened (Smo) gain-of-function” refers to an aberrant modification or mutation of a Smo gene or an increase in the level of expression of the Smo gene, which results in a phenotype which resembles contacting a cell with a hedgehog ligand, e.g., aberrant activation of a hedgehog pathway.

### DETAILED DESCRIPTION OF CERTAIN EMBODIMENTS

The inventors have discovered that administering a hedgehog pathway inhibitor to a tissue can alter the tissues morphology, such as, for example, by increasing blood vessel patency, increasing blood vessel density and/or reducing stromal density. Blood flow can also be improved in ischemic tissues upon administering a hedgehog pathway inhibitor to the tissue. Thus, in certain embodiments, hedgehog pathway inhibitors can be employed to increase delivery of an agent (such as a therapeutic agent or an imaging agent) to a tissue and improve imaging of a tissue (such as, for example, via X-rays and ultrasound). In certain embodiments, the hedgehog pathway inhibitors can be employed to promote new blood vessel formation (e.g., angiogenesis) in a tissue.

### Hedgehog Pathway Inhibitors

The hedgehog pathway inhibitor can be any agent (e.g., small molecule, antibody, small interfering RNA, etc) that exerts its inhibitory affect on the pathway through an interaction with one or more components of the pathway, e.g., the hedgehog ligand, smoothened, patched, or Gli.

Suitable hedgehog inhibitors include, for example, those described and disclosed in U.S. Pat. No. 7,230,004, U.S. Patent Application Publication No. 2008/0293754, U.S. Patent Application Publication No. 2008/0287420, and U.S. Patent Application Publication No. 2008/0293755, the entire disclosures of which are incorporated by reference herein.

Examples of other suitable hedgehog inhibitors include those described in U.S. Patent Application Publication Nos. US 2002/0006931, US 2007/0021493 and US 2007/0060546, and International Application Publication Nos. WO 2001/19800, WO 2001/26644, WO 2001/27135, WO 2001/49279, WO 2001/74344, WO 2003/011219, WO 2003/088970, WO 2004/020599, WO 2005/013800, WO 2005/033288, WO 2005/032343, WO 2005/042700, WO 2006/028958, WO 2006/050351, WO 2006/078283, WO 2007/054623, WO 2007/059157, WO 2007/120827, WO 2007/131201, WO 2008/070357, WO 2008/110611, WO 2008/112913, and WO 2008/131354.

In certain embodiments, the hedgehog pathway inhibitor is represented by a compound selected from the group consisting of Formula I, Formula II, and Formula III:

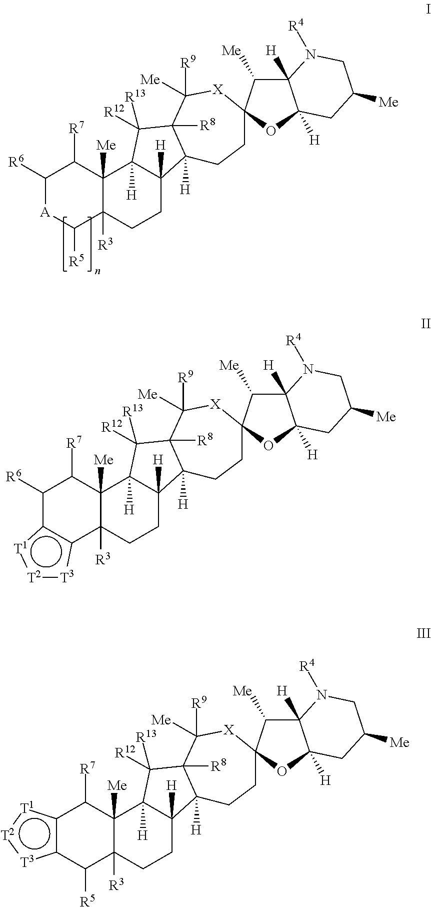

or a pharmaceutically acceptable salt thereof;

wherein A is:

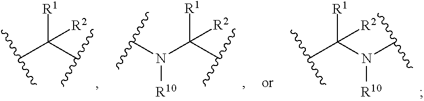

n is 0 or 1;

X is a bond or —CH₂—;

R¹ is selected from —H, optionally substituted alkyl, optionally substituted alkenyl, optionally substituted alkynyl, optionally substituted aryl, optionally substituted aralkyl, optionally substituted cycloalkyl, nitrile, optionally substituted heterocycloalkyl, —OR¹⁰, —N(R¹⁰)(R¹⁰), —NR¹⁰SO₂R¹⁰, —N(R¹⁰)CO₂R¹⁰, —N(R¹⁰)C(O)R¹⁰, —OC(O)R¹⁰, and a sugar;

R² is selected from —H, optionally substituted alkyl, optionally substituted alkenyl, optionally substituted alkynyl, optionally substituted aryl, optionally substituted aralkyl, optionally substituted cycloalkyl, nitrile, and optionally substituted heterocycloalkyl; or R¹ and R² taken together form ═O, ═S, ═N(OR), ═N(R)—, ═N(NR₂), ═C(R)₂;

R³ and R⁵, are, independently, selected from —H, optionally substituted alkyl, optionally substituted aralkyl, optionally substituted alkenyl, and optionally substituted alkynyl; or R³ and R⁵ taken together form a bond;

R⁶ and R⁷ are, independently, selected from —H, optionally substituted alkyl, optionally substituted aralkyl, optionally substituted alkenyl, and optionally substituted alkynyl; or R⁶ and R⁷ taken together form a bond;

R⁸ and R⁹ taken together form a bond;

R⁴ is selected from —H, optionally substituted alkyl, optionally substituted alkenyl, optionally substituted alkynyl, optionally substituted aryl, optionally substituted aralkyl, optionally substituted cycloalkyl, optionally substituted heterocycloalkyl, optionally substituted aralkyl, optionally substituted heteroaryl, optionally substituted heteroaralkyl, optionally substituted haloalkyl, —OR¹⁰, —C(O)R¹⁰, —CO₂R¹⁰, —SO₂R¹⁰, —C(O)N(R¹⁰)(R¹⁰), —[C(R)₂]_{q}—R¹⁰, —[(W)—N(R¹⁰)C(O)]_{q}R¹⁰, —[(W)—C(O)]_{q}R¹⁰, —[(W)—C(O)O]_{q}R¹⁰, —[(W)—OC(O)]_{q}R¹⁰, —[(W)—SO₂]_{q}R¹⁰, —[(W)—N(R¹⁰)SO₂]_{q}R¹⁰, —[(W)—C(O)N(R¹⁰)]_{q}R¹⁰, —[(W)—O]_{q}R¹⁰, —[(W)—N(R)]_{q}R¹⁰, and —[(W)—S]_{q}R¹⁰;

each q, independently, for each occurrence, is 1, 2, 3, 4, 5, or 6;

each R¹⁰ is, independently, selected from —H, optionally substituted alkyl, optionally substituted alkenyl, optionally substituted aralkyl, optionally substituted alkynyl, optionally substituted aryl, optionally substituted cycloalkyl, optionally substituted heterocycloalkyl, optionally substituted aralkyl, optionally substituted heteroaryl, optionally substituted heteroaralkyl and —[C(R)₂]ₚ—R¹¹; wherein p is 0-6; or any two occurrences of R¹⁰ on the same substituent can be taken together to form a 4-8 membered optionally substituted ring which contains 0-3 heteroatoms selected from nitrogen, oxygen, sulfur, and phosphorus;

each R¹¹ is, independently, selected from hydroxyl, —N(R)COR, —N(R)C(O)OR, —N(R)SO₂(R), —C(O)N(R)₂, —OC(O)N(R)(R), —SO₂N(R)(R), —N(R)(R), —COOR, —C(O)N(OH)(R), —OS(O)₂OR, —S(O)₂OR, —S(O)₂R, —OP(O)(OR)(OR), —NP(O)(OR)(OR), and —P(O)(OR)(OR);

each R is, independently, selected from —H, optionally substituted alkyl, optionally substituted alkenyl, optionally substituted alkynyl, optionally substituted aryl, optionally substituted cycloalkyl and optionally substituted aralkyl;

R¹² and R¹³ are, independently, selected from —H, optionally substituted alkyl, optionally substituted alkenyl, optionally substituted alkynyl, optionally substituted aryl, optionally substituted aralkyl, optionally substituted cycloalkyl, nitrile, optionally substituted heterocycloalkyl, —OR¹⁰, —N(R¹⁰)(R¹⁰), —NR¹⁰SO₂R¹⁰, —N(R¹⁰)CO₂R¹⁰, —N(R¹⁰)C(O)R¹⁰, and —OC(O)R¹⁰; or R¹² and R¹³ taken together form ═O, ═S, ═N(OR), ═N(R)—, ═N(NR₂), ═C(R)₂;

each W is, independently for each occurrence, selected from an optionally substituted alkyl diradical, optionally substituted alkenyl diradical, optionally substituted alkynyl diradical, optionally substituted aryl diradical, optionally substituted cycloalkyl diradical, optionally substituted heterocycloalkyl diradical, optionally substituted aralkyl diradical, optionally substituted heteroaryl diradical and an optionally substituted heteroaralkyl diradical;

and

T¹-T²-T³ is selected from Y-B-A, B-Y-A, and A-B-Y; wherein each of A and B is, independently, selected from nitrogen, sulfur and —C(R¹⁴)₂— and Y is selected from —O—, —S—, and —N(R¹⁵)—;

R¹⁴ is, independently, selected from —H, optionally substituted alkyl, optionally substituted alkenyl, optionally substituted alkynyl, optionally substituted cycloalkyl, optionally substituted aryl, optionally substituted aralkyl, optionally substituted heteroaryl, optionally substituted heteroaralkyl, perhaloalkyl, halo, nitro, nitrile, ═O, —SR¹⁰, —OR¹⁰, —N(R¹⁰)(R¹⁰), —C(O)R¹⁰, —CO₂R¹⁰, —OC(O)R¹⁰, —C(O)N(R¹⁰)(R¹⁰), —N(R¹⁰)C(O)R¹⁰, —N(R¹⁰)C(O)N(R¹⁰)(R¹⁰), —S(O)R¹⁰, —S(O)₂R¹⁰, —S(O)₂N(R¹⁰)(R¹⁰), —N(R¹⁰)S(O)₂R¹⁰ and —[C(R¹⁰)₂]_{q}—R¹¹; and

R¹⁵ is selected from —H, optionally substituted alkyl, optionally substituted alkenyl, optionally substituted alkynyl, optionally substituted cycloalkyl, optionally substituted aryl, optionally substituted aralkyl, optionally substituted heteroaryl, optionally substituted heteroaralkyl, perhaloalkyl, —C(O)R¹⁰, —CO₂R¹⁰, —C(O)N(R¹⁰)(R¹⁰), —S(O)R¹⁰, —S(O)₂R¹⁰, —S(O)₂N(R¹⁰)(R¹⁰), and —[C(R)₂]_{q}—R¹¹.

In certain embodiments, the hedgehog pathway inhibitor is a compound of Formula I.

For example, in certain embodiments, A is

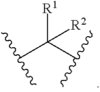

In certain embodiments, X is —CH₂—.

In certain embodiments, R¹ is, —OR¹⁰, —N(R¹⁰)(R¹⁰), —NR¹⁰SO₂R¹⁰, —N(R¹⁰)CO₂R¹⁰, —N(R¹⁰)C(O)R¹⁰, or —OC(O)R¹⁰. In certain embodiments, R¹ is —NR¹⁰SO₂R¹⁰.

In certain embodiments, R² is —H or optionally substituted alkyl. In certain embodiments, R² is —H.

In certain embodiments, R¹ and R² taken together form ═O.

In certain embodiments, R³ and R⁵ are —H or R³ and R⁵ form a bond.

In certain embodiments, R⁶ and R⁷ are —H or R⁶ and R⁷ form a bond.

In certain embodiments, R¹² and R¹³ are —H.

In certain embodiments, R⁴ is selected from —H, optionally substituted aryl, optionally substituted heterocycloalkyl, optionally substituted heteroaryl, —OR¹⁰, —C(O)R¹⁰, —CO₂R¹⁰, —SO₂R¹⁰, and —C(O)N(R¹⁰)(R¹⁰). In certain embodiments, R⁴ is selected from —H, —OR¹⁰, —C(O)R¹⁰, —CO₂R¹⁰, —SO₂R¹⁰, and —C(O)N(R¹⁰)(R¹⁰). In certain embodiments, R⁴ is —H.

In certain embodiments, each R¹⁰ is, independently for each occurrence, selected from —H, optionally substituted alkyl, optionally substituted alkenyl, optionally substituted aralkyl, optionally substituted alkynyl, optionally substituted aryl, optionally substituted cycloalkyl, optionally substituted heterocycloalkyl, optionally substituted heteroaryl, optionally substituted heteroaralkyl and —[C(R)₂]ₚ—R¹¹. In certain embodiments, each R¹⁰ is, independently for each occurrence, selected from —H, optionally substituted alkyl, optionally substituted aralkyl, optionally substituted aryl, optionally substituted heteroaryl, and optionally substituted heteroaralkyl. In certain embodiments, each R¹⁰ is —H. In certain embodiments, each R¹¹ is —H. In certain embodiments, n is 1.

In certain embodiments, a compound of Formula I has the Formula I-A:

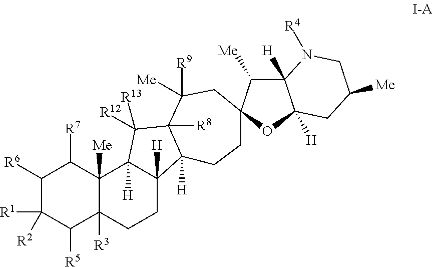

In certain embodiments, a compound of Formula I has the Formula I-B:

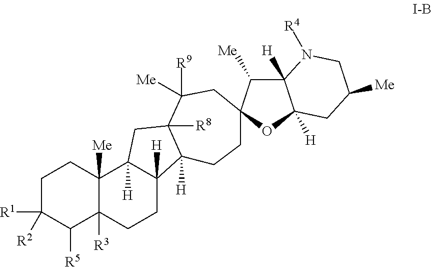

In certain embodiments, a compound of Formula I has the Formula I-C:

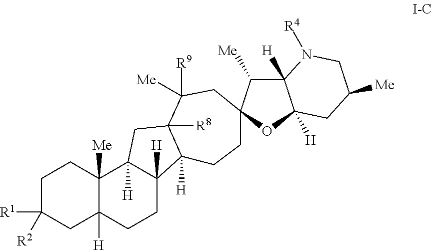

In certain embodiments, a compound of Formula I has the Formula I-D:

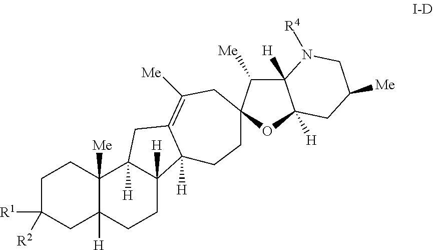

Exemplary compounds of Formula I include, but are not limited to, compounds of Table 1:

TABLE 1

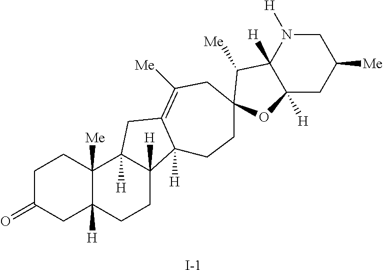

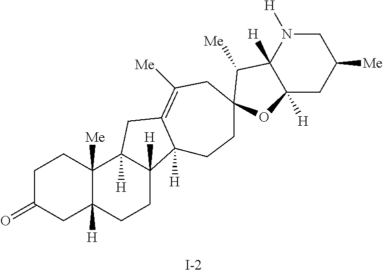

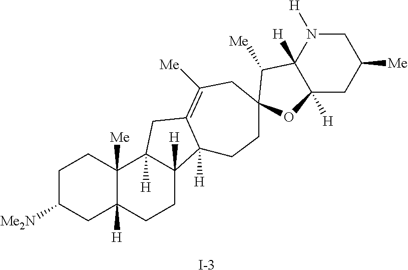

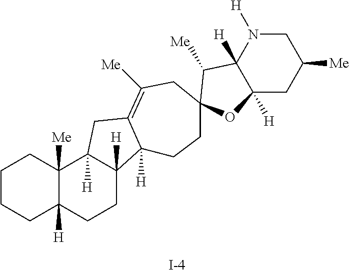

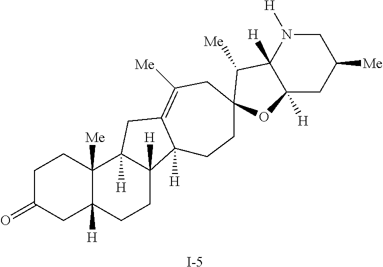

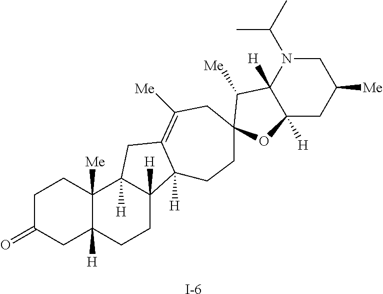

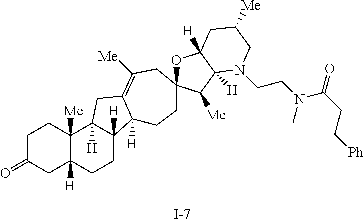

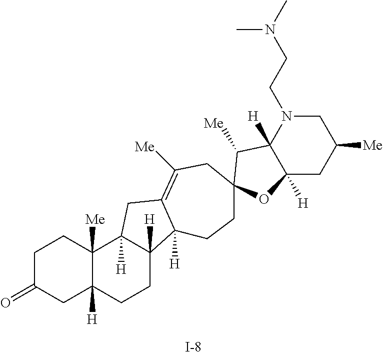

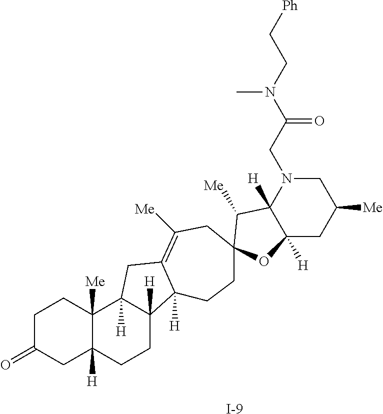

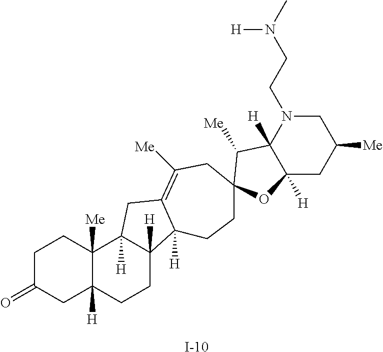

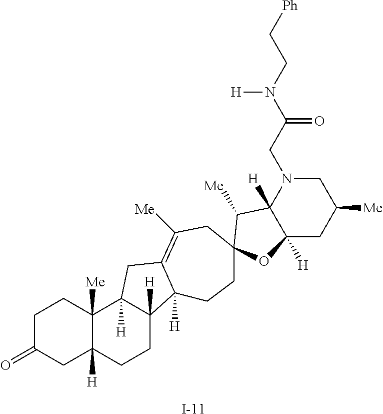

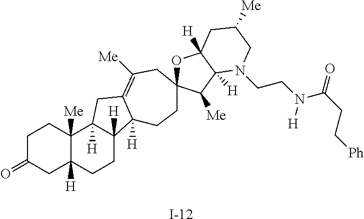

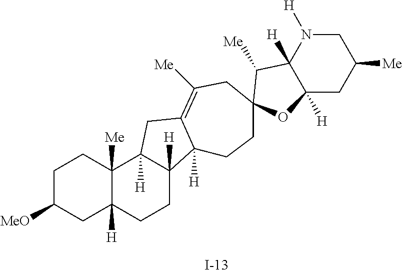

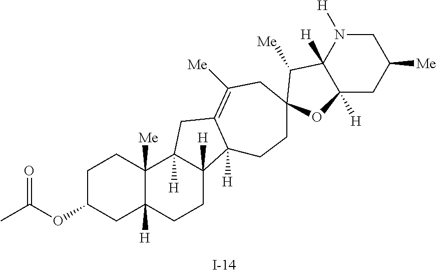

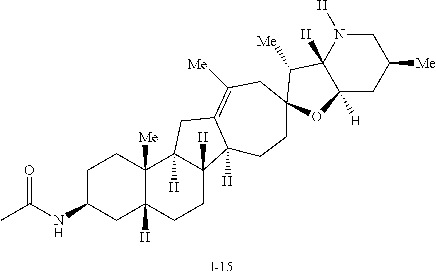

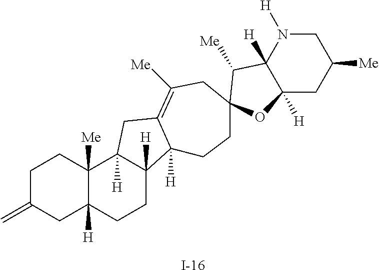

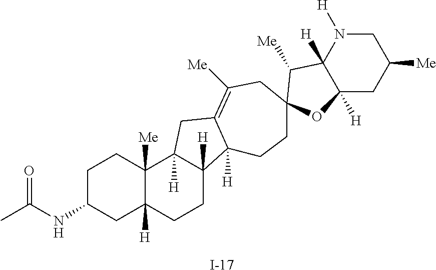

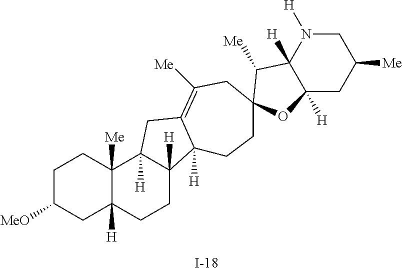

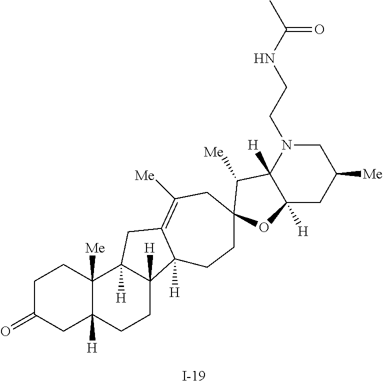

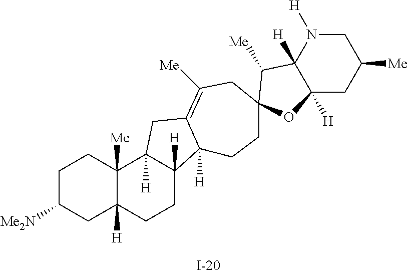

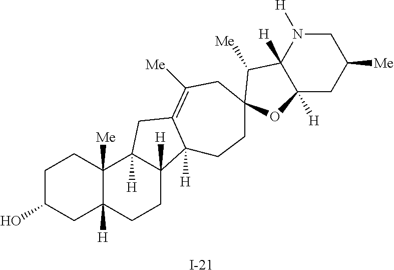

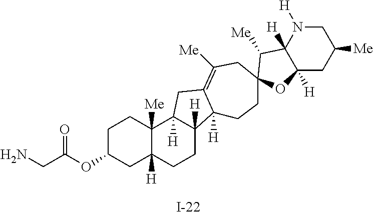

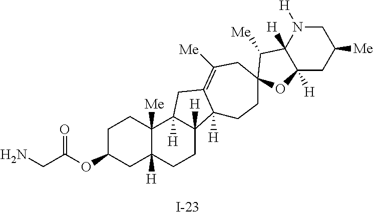

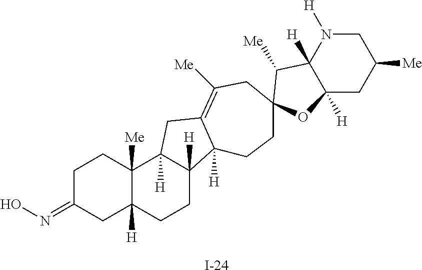

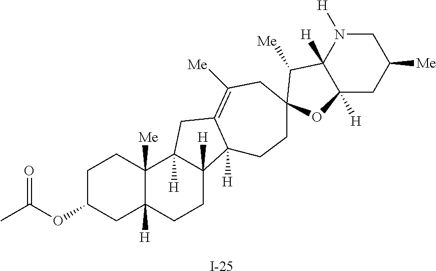

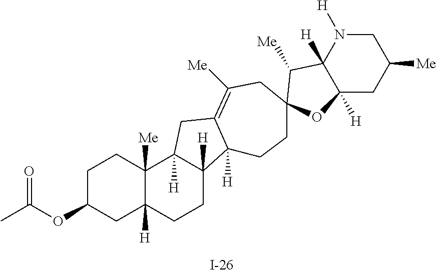

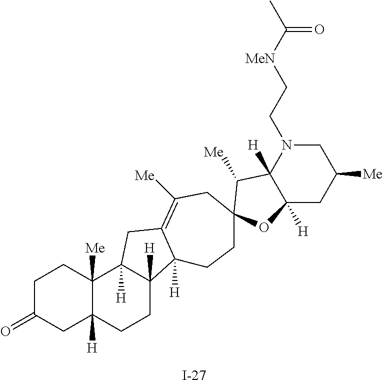

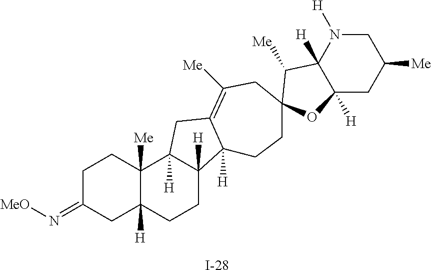

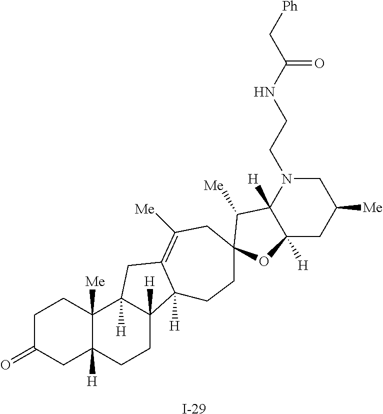

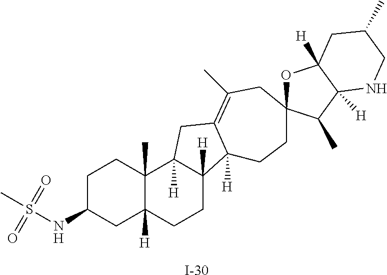

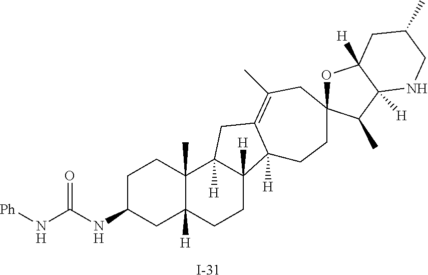

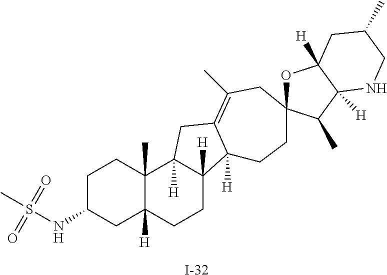

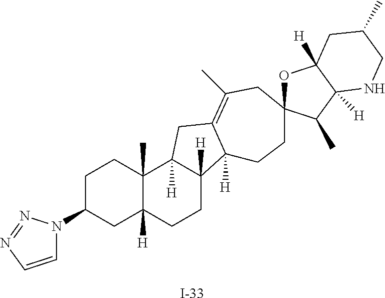

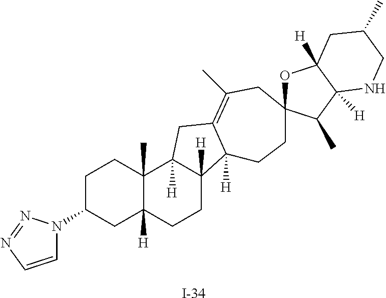

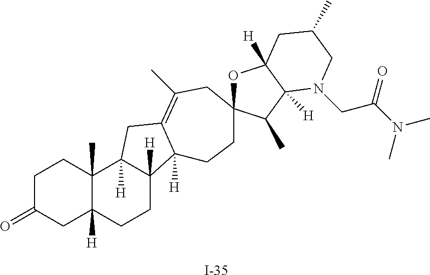

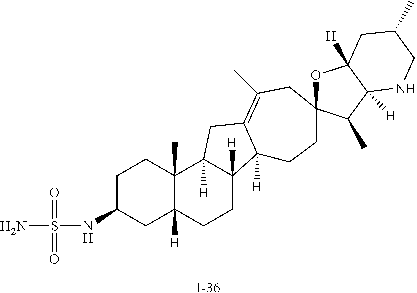

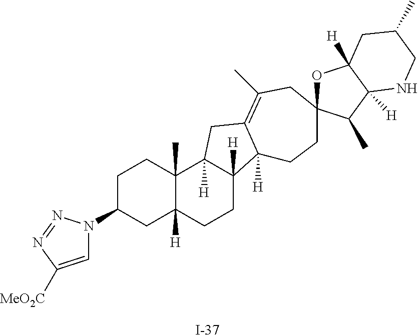

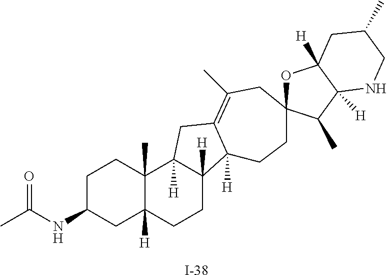

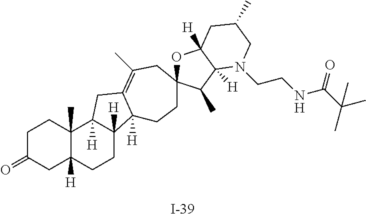

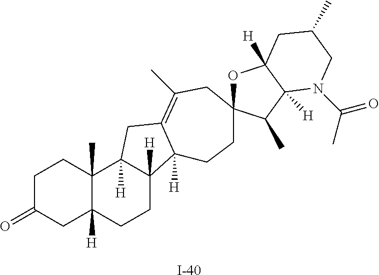

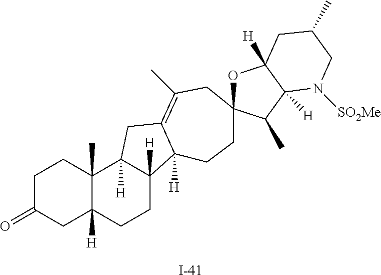

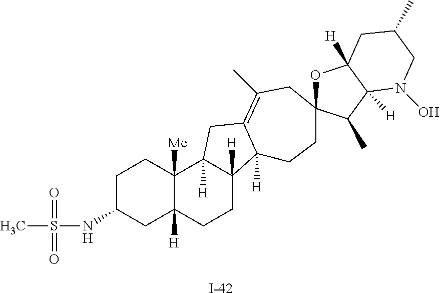

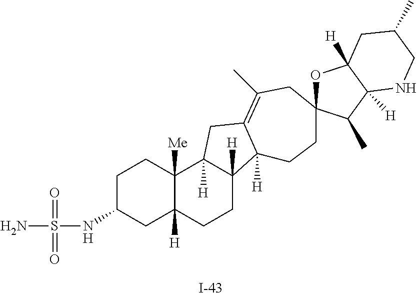

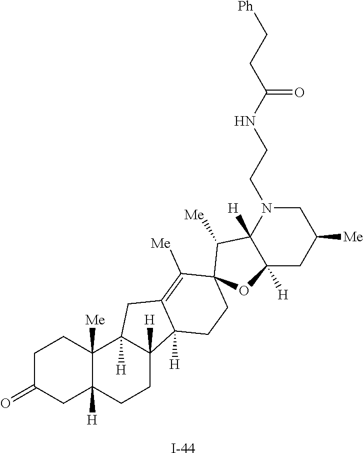

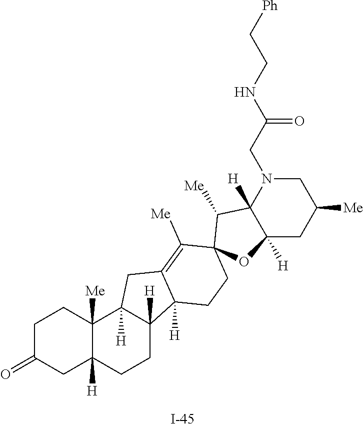

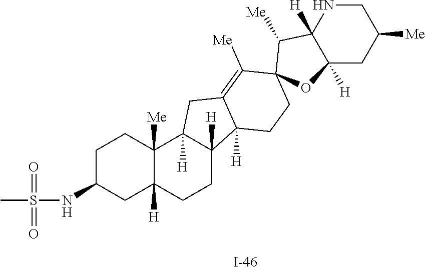

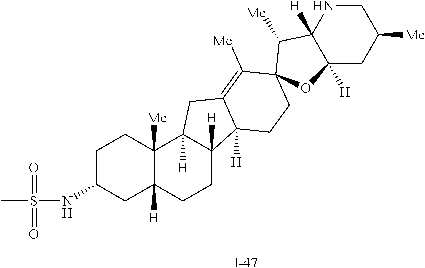

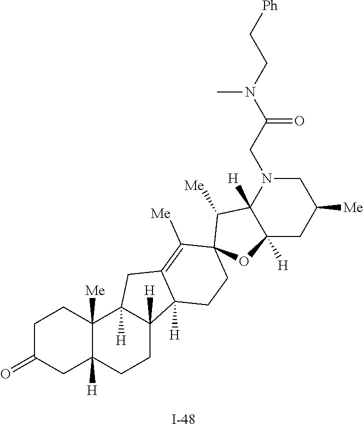

and pharmaceutically acceptable salts thereof.

Exemplary compounds of Formula I also include, but are not limited to, compounds of Table 2:

TABLE 2

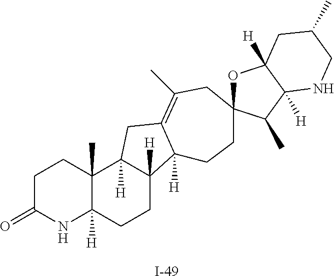

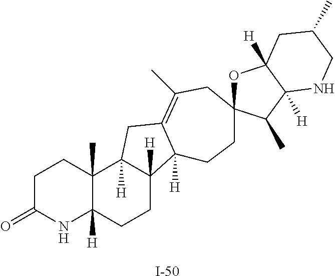

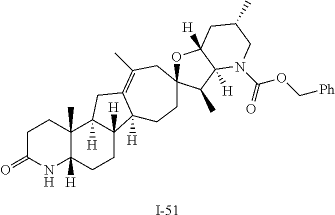

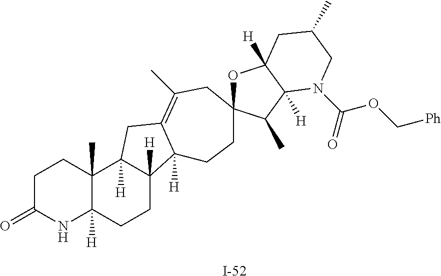

and pharmaceutically acceptable salts thereof.

Exemplary compounds of Formula I also include, but are not limited to, compounds of Table 3:

TABLE 3

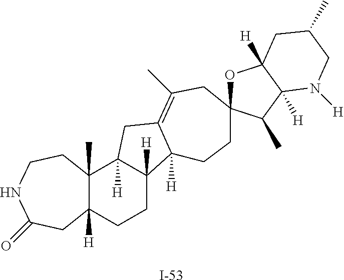

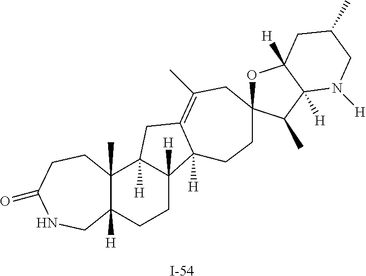

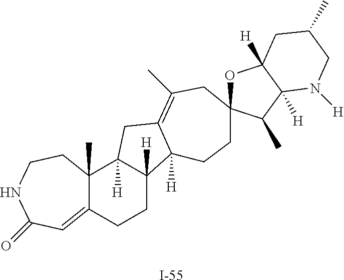

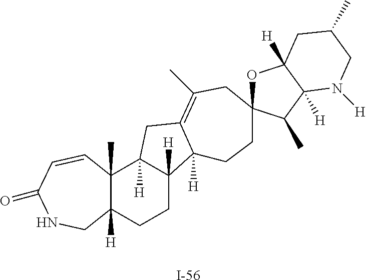

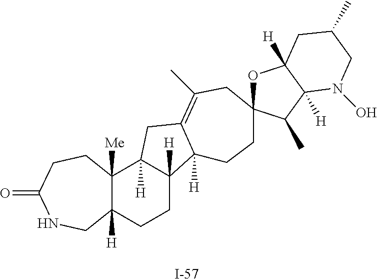

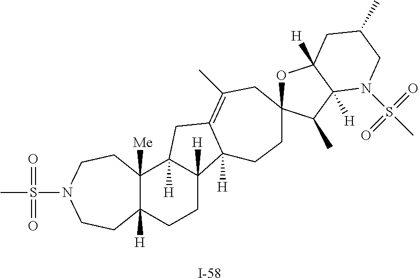

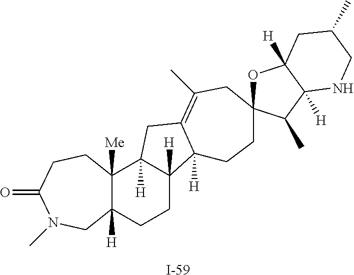

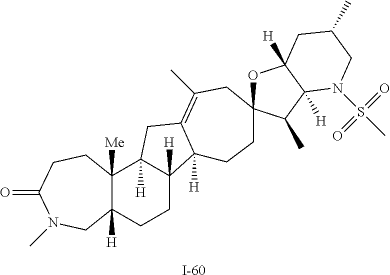

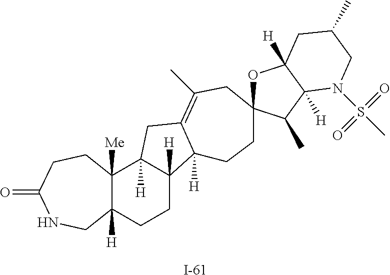

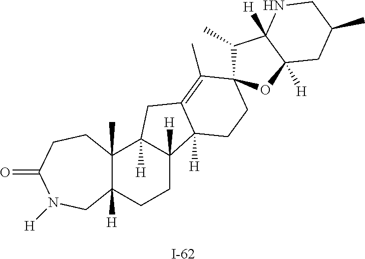

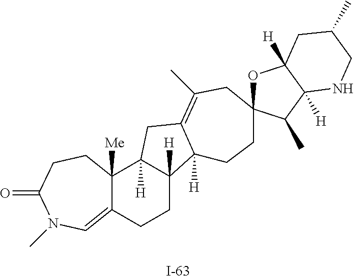

and pharmaceutically acceptable salts thereof.

Exemplary compounds of Formula II include, but are not limited to, the compound of Table 4:

TABLE 4

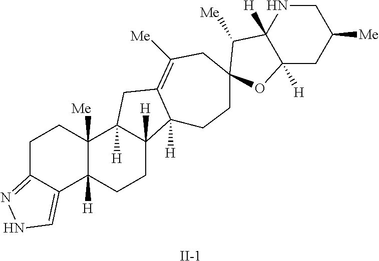

and tautomers and/or pharmaceutically acceptable salts thereof.

Exemplary compounds of Formula III include, but are not limited to, compounds of Table 5:

TABLE 5

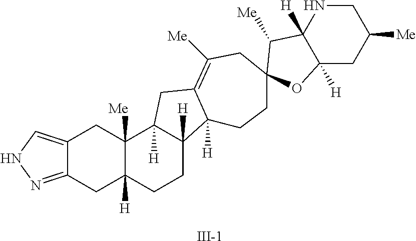

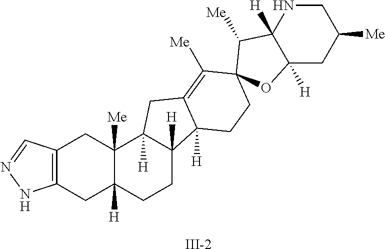

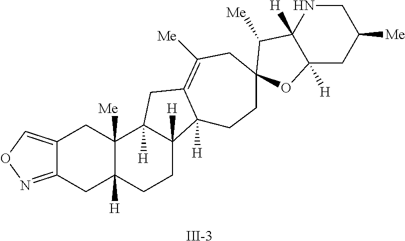

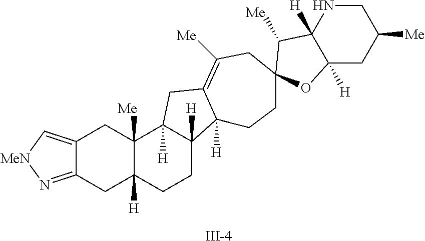

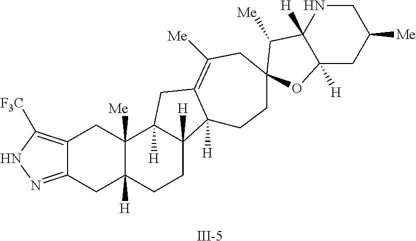

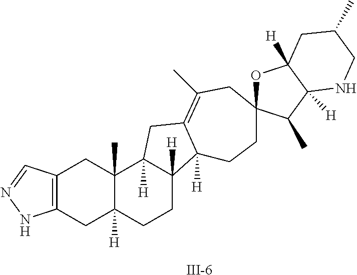

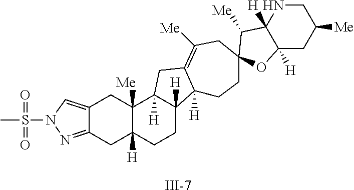

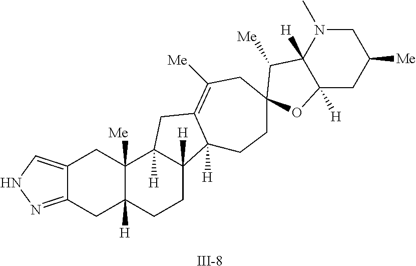

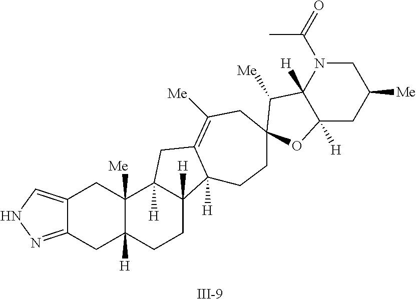

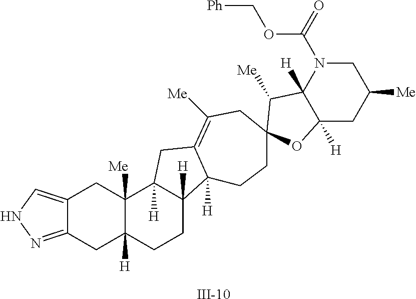

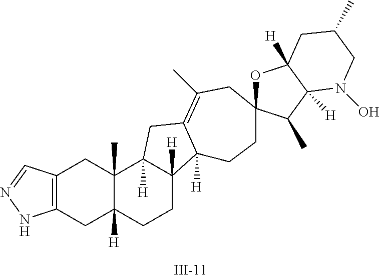

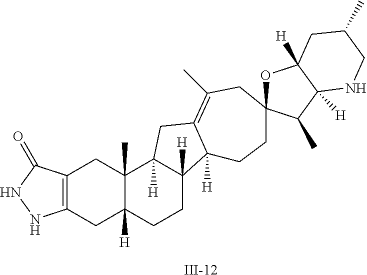

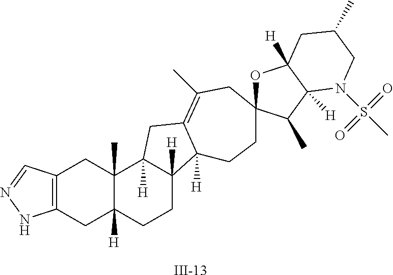

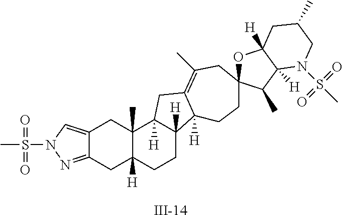

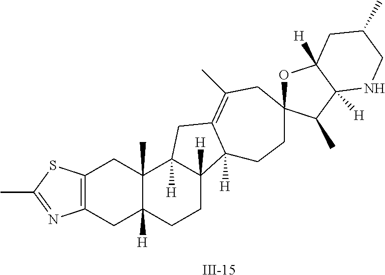

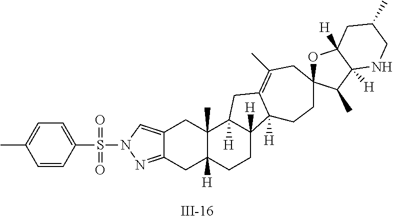

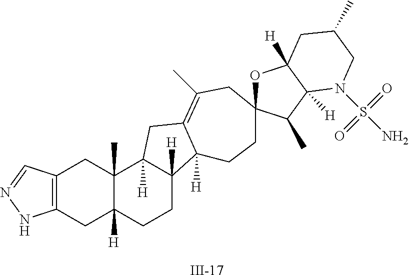

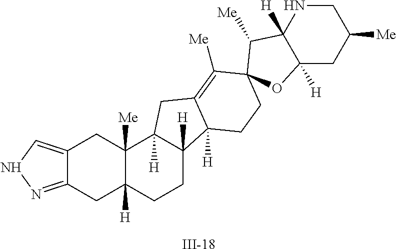

and tautomers and/or pharmaceutically acceptable salts thereof.

In certain embodiments, the hedgehog pathway inhibitor is a compound of Formula I.

In certain embodiments, the hedgehog pathway inhibitor is a compound as provided in Table 1, or a pharmaceutically acceptable salt thereof.

In certain embodiments, the hedgehog pathway inhibitor is compound I-32, or a pharmaceutically acceptable salt thereof.

### Methods of Use and Treatment

As generally described above and herein, hedgehog pathway inhibitors can be used to improve delivery of an agent, such as a therapeutic or imaging agent, to a tissue.

Thus, in one aspect, the present invention provides a method of increasing delivery of an agent (e.g., a therapeutic agent or an imaging agent) to a tissue, comprising administering a hedgehog pathway inhibitor and the agent to the tissue. In certain embodiments, the method further comprises administering one or more additional agents, such as a second, third, fourth, fifth, etc. agent, to the tissue.

For example, in certain embodiments, the present invention provides a method of imaging a tissue, comprising administering a hedgehog pathway inhibitor and an imaging agent to said tissue and using said imaging agent to image the tissue. In other embodiments, the present invention provides a method of increasing delivery of a therapeutic agent (e.g., a chemotherapeutic agent) to a tissue (e.g., a tumor or cancerous tissue) comprising administering a hedgehog pathway inhibitor and the therapeutic agent to said tissue.

In another aspect, provided are methods of altering tissue morphology (e.g., reducing stromal density, increasing blood vessel density and/or increasing blood vessel patency) in a tissue. Such methods comprise administering a hedgehog pathway inhibitor to a tissue. In certain embodiments, the method further comprises administering an agent (e.g., a therapeutic agent or an imaging agent) to the tissue.

For example, in certain embodiments, the present invention provides a method of reducing the stromal density in a tissue, comprising administering a hedgehog pathway inhibitor to the tissue. In certain embodiments, the method further comprises administering an agent (e.g., a therapeutic agent or an imaging agent) to the tissue. Stromal cells can include fibroblasts, immune cells, pericytes, endothelial cells, and inflammatory cells, as well as other cells present in the tumor but not derived from the initiating neoplastic cell. For example, in certain embodiments, the method of reducing stromal density comprises reducing the fibroblast (i.e., fibroblast and/or fibrocyte) content in a tissue. In certain embodiments, the fibroblast is a tumor-related fibroblast. In certain embodiments, the fibroblast is a non-tumor-related fibroblast. In certain embodiments, the fibroblast is a tumor-related fibroblast and the agent is a chemotherapeutic. In some embodiments, the method of reducing the stromal density in a tissue can be used to treat cancer (for example, breast cancer, ovarian cancer, prostate cancer, pancreatic cancer, gastrointestinal tract cancer, lung cancer, or squamous cell carcinomas) by administering a hedgehog pathway inhibitor and a chemotherapeutic agent.

In certain embodiments, the present invention provides a method of increasing blood vessel density in a tissue, comprising administering a hedgehog pathway inhibitor to said tissue. In certain embodiments, the method further comprises administering an agent (e.g., a therapeutic agent or an imaging agent) to the tissue. In some embodiments, the method of increasing blood vessel density in a tissue can be used to treat cancer (for example, breast cancer, ovarian cancer, prostate cancer, pancreatic cancer, gastrointestinal tract cancer, lung cancer, or squamous cell carcinomas) by administering a hedgehog pathway inhibitor and a chemotherapeutic agent.

In certain embodiments, the present invention provides a method of increasing blood vessel patency in a tissue, comprising administering a hedgehog pathway inhibitor to said tissue. In certain embodiments, the method further comprises administering an agent (e.g., a therapeutic agent or an imaging agent) to the tissue. In some embodiments, the method of increasing blood vessel patency in a tissue can be used to treat cancer (for example, breast cancer, ovarian cancer, prostate cancer, pancreatic cancer, gastrointestinal tract cancer, lung cancer, or squamous cell carcinomas) by administering a hedgehog pathway inhibitor and a chemotherapeutic agent.

In certain embodiments, the methods of increasing blood vessel density and/or blood vessel patency can be used to treat ischemia (e.g., ischemia as a result of, tachycardia, atherosclerosis, hypotension, thromboembolism, embolism, and the like) in, e.g., a limb, heart, brain, etc. The blood vessel can be any type of blood vessel, including for example, arteries, arterioles, capillaries, venules, and veins. In certain embodiments the blood vessel is a microvessel.

In certain embodiments, the hedgehog pathway inhibitor can be used to promote the growth of new blood vessels from pre-existing vessels (i.e., angiogenesis). Thus, in certain embodiments, the present invention provides a method of promoting angiogenesis in a tissue, comprising administering a hedgehog pathway inhibitor to said tissue. In certain embodiments, the method further comprises administering an agent (e.g., a therapeutic agent or an imaging agent) to the tissue.

In certain embodiments, the invention provides methods for treating (e.g., reducing the amount or occurrence of) or preventing tumor metastasis, comprising administering to a mammal in need thereof a hedgehog pathway inhibitor and a chemotherapeutic agent. In certain embodiments, the hedgehog pathway inhibitor and chemotherapeutic agent are administered concurrently. In certain embodiments, the hedgehog pathway inhibitor and chemotherapeutic agent are administered sequentially. In certain embodiments, the tumor is a pancreatic tumor, a prostate tumor, a breast tumor, a desmoplastic small round cell tumor, a colon tumor, an ovarion tumor, a bladder tumor, or an osteocarcinoma.

### (a) Administration

As used herein, “administration” or “administering” refers to the contact of one or more components (i.e., a hedgehog pathway inhibitor and, optionally, a first, second, third, fourth, fifth etc. agent) to a tissue. Administration comprises in vivo administration (e.g., orally, parenterally, topically, intravaginally, intrarectally, sublingually, ocularly; transdermally, pulmonarily, nasally, etc. administering to a mammal one or more components provided in one or more pharmaceutical compositions) or in vitro administration (e.g., contacting one or more components to a cell culture or tissue culture). In vivo administration comprises administration of a hedgehog pathway inhibitor and, optionally, an agent (e.g., a therapeutic agent or an imaging agent) to a mammal (e.g., such as a human, a primate, a canine, a feline, or a rodent), wherein the mammal is in need of such treatment.

In certain embodiments, wherein the hedgehog inhibitor is administered in combination with an agent, the hedgehog pathway inhibitor and the agent are administered either concurrently or sequentially.

Sequential administration refers to the administration of a first component over a period of time, stopping the administration of the first component, followed by administration of a second component. For example, sequential administration includes administration of a hedgehog pathway inhibitor, stopping the administration of the hedgehog pathway inhibitor, followed by administration of the agent. Sequential administration also includes administration of an agent, stopping the administration of the agent, followed by administration of a hedgehog pathway inhibitor. Once administration of the first component is stopped, the second component can be administered immediately after stopping administration of the first component, or the second component can be administered after an effective time period after stopping administration of the first component.

Concurrent administration (e.g., simultaneously in time; “co-administration”) refers to administration of a first component and a second component over the same time period. For example, concurrent administration includes administering a first component over a period of time and then administering a second component together with the first component. Concurrent administration also includes administering the first component and the second component for an effective period of time and then stopping the administration of either the first or second component and continuing the administration of the remaining component. Concurrent administration also includes administering the first component and the second component for an effective period of time and then stopping the administration of both the first and second component.

An effective time period can be an amount of time to give a benefit from the administration of the first and/or second component.

In certain embodiments, wherein the hedgehog pathway inhibitor is administered with an agent, the hedgehog pathway inhibitor is administered to a mammal twice a day, once a day, once a week, twice a week, or three times a week, for up to about 1 day before, about three days before, five days before, about one week, about two weeks, about three weeks, or about four weeks prior to the initiating dosing of the agent.

In certain embodiments, wherein the hedgehog pathway inhibitor is administered with an agent, the hedgehog pathway inhibitor is administered to a mammal from about 3 days to about 10 days, from about 7 days to about 14 days, or from about 10 days to about 20 days prior to initiating administration of the agent. Administration of the hedgehog pathway inhibitor can be terminated when the administration of the agent is initiated or the hedgehog pathway inhibitor can be administered concurrently, for any amount of time, with the agent. In certain instances, the hedgehog pathway inhibitor is dosed for about 7 days, about 14 days, or about 21 days. At any of these points, dosing of the hedgehog pathway inhibitor may be terminated and dosing of the agent can be initiated.

### (b) Tissues

As used herein, “tissue” refers any tissue type; for example, an ischemic tissue, tumor tissue, non-tumor tissue, and/or poorly permeable tissue. In certain embodiments, the tumor tissue is hypoxic. In certain embodiments, the tissue is characterized as exhibiting Hedgehog pathway activation. In certain embodiments, the Hedgehog pathway activation is characterized by one or more phenotypes selected from group consisting of a Patched (Ptc) loss-of-function phenotype or a Smoothened (Smo) gain-of-function phenotype. Exemplary tissues include, but are not limited to, cardiac tissue, brain tissue, connective tissue, muscle tissue, nervous tissue and epithelial tissue. Examples of connective tissue include, but are not limited to areola tissue, adipose tissue, recticular tissue, regular tissue, irregular tissue, elastic tissue, hyaline tissue, fibrocartilage tissue, elastic tissue, bone, blood, and lymphatic tissue. Examples of muscle tissue include, but are not limited to skeletal muscle tissue, smooth muscle tissue (e.g., smooth muscle found in the walls of the stomach, intestines, bronchi, uterus, urethra, bladder, blood vessels, and skin), and cardiac muscle tissue. Examples of nervous tissue include, but are not limited to unipolar neurons, bipolar neurons, and multipolar neurons. Examples of epithelial tissue include, but are not limited to squamous epithelial tissue, cuboidal epithelial tissue, columnar epithelial tissue, and pseudostratified epithelial tissue. The hedgehog pathway inhibitor can be contacted with the tissue in vitro or in vivo.

The tissue to be treated can be tumor/cancerous tissue or non-cancerous tissue. Tumor tissues that can be treated using the methods described herein includes, but are not limited to, basal cell carcinoma, neuroectodermal tumor, medulloblastoma, pancreatic cancer, esophageal cancer, gastric cancer, lung cancer (e.g., non-small cell lung cancer, small cell lung cancer), breast cancer, ovarian cancer, cervical cancer, testicular cancer, prostate cancer, pancreatic cancer, hepatocellular cancer, skin cancer, gastrointestinal tract (GIST) cancer, lung cancer, squamous cell carcinoma, colorectal cancer, colon cancer, stomach cancer, desmoplastic small round cell tumor, bladder cancer, and osteocarcinoma. In certain embodiments, the cancer is pancreatic cancer.

In other embodiments, a tumor tissue can be any cancerous tissue/tumor characterized by excessive amounts of desmoplastic stroma, e.g., breast cancer, ovarian cancer, prostate cancer, pancreatic cancer, gastrointestinal tract cancer, lung cancer, and squamous cell carcinomas. In certain embodiments, the cancer is pancreatic cancer.

The tissue can also comprise an autochthonous tumor tissue. For example, in certain embodiments, the present invention provides a method for treating an autochthonous tumor in a mammal, comprising administering a hedgehog pathway inhibitor and a chemotherapeutic agent to said mammal.

Autochthonous tumors include tumors that are generated spontaneously, e.g., by germline mutation(s) and/or somatic mutation(s), or induced artificially by, e.g., chemical and/or genetic manipulation. In certain instances, autochthonous tumors include the metastasis (e.g., a bone metastasis) of such spontaneously generated and artificially induced tumors. Autochthonous tumors do not include xenograft tumors.

Autochthonous tumor tissues and/or vasculature morphology can be very different from those of ecotopic tumors, i.e., tumor xenografts. In certain instances, autochthonous tumors are characterized by prominent acellular and cellular stromal components, whereas ecotopic tumors can contain very little stroma. The transit of blood through the autochthonous tumor microvasculature can be impaired by abnormal structures, elevated interstitial fluid pressure, and leaky capillaries, which may not be present in ecotopic tumors, or may be present in a conformation that does not reflect the typical physiology of human tumors. Such impaired vascular function, can reduce the delivery of therapeutic agents to the tumor. Thus, the delivery of agents, e.g., chemotherapeutic agents, to an autochthonous tumor can be improved by co-administering a hedgehog pathway inhibitor. In certain embodiments, the autochthonous tumor is a tumor exhibiting Hedgehog pathway activation. In certain embodiments, the Hedgehog pathway activation is characterized by one or more phenotypes selected from group consisting of a Patched (Ptc) loss-of-function phenotype or a Smoothened (Smo) gain-of-function phenotype.

### (c) Therapeutic Agents

In certain embodiments, the method comprises administering a hedgehog pathway inhibitor and a therapeutic agent to a tissue. In certain embodiments, the method further comprises one or more additional therapeutic agents, such as a second, third, fourth, fifth, etc. therapeutic agent. Hedgehog pathway inhibitors can be used to improve the penetration of the therapeutic agent in the tissue, e.g., dense tissues, cancerous tissues. In certain embodiments, the tissue is a tumor tissue/cancerous tissue, as described above and herein. In certain embodiments, the tumor tissue is hypoxic. In certain embodiments, the therapeutic agent is an agent useful in the treatment of cancer.

For example, in certain embodiments, the therapeutic agent is radiation. Restored vasculature increases perfusion to an extent that hypoxia of the tumor tissue is diminished, and, in such instances, the tumor can become sensitized to radiation. Radiation useful in the methods described herein can be administered in a variety of fashions. For example, radiation may be electromagnetic or particulate in nature. Electromagnetic radiation useful in the methods described herein include, but is not limited to, x-rays and gamma rays. Particulate radiation useful in the methods described herein include, but is not limited to, electron beams, proton beams, neutron beams, alpha particles, and negative pi mesons. The radiation may be delivered using conventional radiological treatment apparatus and methods, and by intraoperative and stereotactic methods. Additional discussion regarding radiation treatments suitable for use in methods described herein may be found throughout Leibel et al., Textbook of Radiation Oncology, W. B. Saunders Co. (1998), and in Chapters 13 and 14 of that text. Radiation may also be delivered by other methods such as targeted delivery, for example by radioactive seeds, or by systemic delivery of targeted radioactive conjugates.

In certain embodiments, the therapeutic agent is a chemotherapeutic agent. Chemotherapeutic agents include, but are not limited to, small molecules, antibodies, small interfering RNA, etc. For example, chemotherapeutic agents include, but are not limited to, gemcitabine, methotrexate, taxol, mercaptopurine, thioguanine, hydroxyurea, cytarabine, cyclophosphamide, ifosfamide, nitrosoureas, mitomycin, dacarbazine, procarbizine, etoposides, prednisolone, dexamethasone, cytarbine, campathecins, bleomycin, doxorubicin, idarubicin, daunorubicin, dactinomycin, plicamycin, mitoxantrone, asparaginase, nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g., dacarbazine and temozolomide), platinum containing compounds (e.g., cisplatin, carboplatin, and oxaliplatin), vinca alkaloids (e.g., vincristine, vinblastine, vindesine, and vinorelbine), taxoids (e.g., paclitaxel, docetaxol, albumin-bound paclitaxel), epi podophyllins (e.g., etoposide, teniposide, topotecan, 9-aminocamptothecin, camptoirinotecan, crisnatol, and mytomycin C), anti-metabolites, DHFR inhibitors (e.g., methotrexate and trimetrexate), IMP dehydrogenase inhibitors (e.g., mycophenolic acid, tiazofurin, ribavirin, and EICAR), ribonucleotide reductase inhibitors (e.g., hydroxyurea and deferoxamine), uracil analogs (e.g., fluorouracil, floxuridine, doxifluridine, ratitrexed, and capecitabine), cytosine analogs (e.g., cytarabine (ara C), cytosine arabinoside, and fludarabine), purine analogs (e.g., mercaptopurine and thioguanine), anti-estrogens (e.g., tamoxifen, raloxifene, and megestrol), LHRH agonists (e.g., goserelin and leuprolide acetate), anti-androgens (e.g., flutamide and bicalutamide), vitamin D3 analogs (e.g., EB 1089, CB 1093, and KH 1060), photodyamic therapies (e.g., vertoporfin (BPD-MA), phthalocyanine, photosensitizer Pc4, and demethoxy-hypocrellin A (2BA-2-DMHA)), cytokines (e.g., interferon α, Interferon γ and tumor necrosis factor), isoprenylation inhibitors (e.g., lovastatin), dopaminergic neurotoxins (e.g., 1-methyl-4-phenylpyridinium ion), cell cycle inhibitors (e.g., staurosporine), actinomycins (e.g., actinomycin D and dactinomycin), bleomycins (e.g., bleomycin A2, bleomycin B2, and peplomycin), anthracyclines (e.g., daunorubicin, doxorubicin (adriamycin), idarubicin, epirubicin, pirarubicin, zorubicin, and mitoxantrone), MDR inhibitors (e.g., verapamil), Ca²⁺ ATPase inhibitors (e.g., thapsigargin), antibodies (e.g., avastin, erbitux, rituxan, and bexxar), corticosteroids (e.g., prednilone and predisone), imatinib, thalidomide, lenalidomide, bortezomib, gemcitabine, erlotinib, gefitinib, sorafenib, sutinib, nilotinib, lapatinib, dasatinib, bevacizumab, cetuximab, trastuzumab, capecitabine, Alimta (pemetrexed), epirubicin, bortezomib, a fluoropyrimidine analog, a nucleoside cytidine analog, a topoisomeraseinhibitor, an antimicrotubule agent, a proteasome inhibitor, a vitamin D analog, an arachidonic acid pathway inhibitor, a histone deacetylase inhibitor (e.g., Vorinostat, Valproic acid) and a farnesyltransferase inhibitor (e.g., tipifarnib, lonafamib).

In certain embodiments, the chemotherapeutic agent is selected from the group consisting of gemcitabine, capecitabine, 5-fluorouracil, floxuridine, doxifluridine, ratitrexed, mitomycin, leucovirin, cisplatin, carboplatin, oxaliplatin, erbitux or erlotinib.

In certain embodiments, the chemotherapeutic agent is gemcitabine.

### (d) Imaging Agents

In certain embodiments, the method comprises administering a hedgehog pathway inhibitor and an imaging agent to the tissue. The methods described herein can be used to image poorly permeable tissues. In certain embodiments, the tissue is a cancerous tissue, as described above and herein. In such instances, the imaging agent can be an agent useful in the treatment/analysis of the cancerous tissue.

The hedgehog pathway inhibitor can alter (e.g., improve) delivery of an imaging agent to a tissue. Imaging agents useful in the methods described herein include, but are not limited to, magnetic resonance imaging (MRI) contrast agents, computerized axial tomography (CAT) contrast agents, and positron emission tomography (PET) contrast agents.

Exemplary MRI contrast agents include, but are not limited to, paramagnetic complexes, such as gadolinium(III), iron(III), mangangese(II), mangangese(III), chromium(III), copper(II), dysprosium(II), terbium(III), terbium(IV), holmium(III), erbium(III), praseodymium(III), europium(II), and europium (III) complexes, and microcrystalline iron oxide compounds.

Exemplary CAT contrast agents include, but are not limited to, bismuth and barium salts, and soluble and insoluble iodinated organic compounds.

Exemplary PET contrast agents include, but are not limited to, any organic or inorganic positron emitting radionuclide. Such radionuclides include, C¹¹, N¹³, O¹⁵, and F¹⁸. In instances where the radionuclide is incorporated into glucose (e.g., 2-fluoro-2-deoxy-D-glucose), the concentrations of the radionuclide tracer in the tissue can be used to monitor tissue metabolic activity.

Diagnostic imaging, for example, contrast ultrasound, X-rays (e.g., fluoroscopy), and photoacoustic imaging, may also be used to evaluate the effect the hedgehog pathway inhibitor has on the tissue.

For example, after administration of a hedgehog inhibitor and an imaging agent (e.g., for example, a small molecule fluorescent probe such as doxorubicin) to a mammal (e.g., a mouse) for a period of time, the tissue of interest can be harvested and confocal microscopy can be used to visualize the perfusion of doxorubicin in the tissue. The tissue can optionally be stained with CD31 antibodies to measure total vascular content of the tissue and the extent of perfusion of the fluorescent probe therein.

In a second example, imaging with contrast ultrasound can be used to evaluate the vascular perfusion of a tissue. Accordingly, after administration of a hedgehog pathway inhibitor for a period of time to a mammal, microbubbles can be administered to the mammal, and contrast ultrasonography can be used to measure tissue perfusion of the microbubbles.

In a third example, after co-administration of a hedgehog pathway inhibitor and a magnetic resonance imaging agent (e.g., gadolinium(III) diethylenetriaminopentaacetic acid) to a mammal for a period of time, the tissue or region of interest in the mammal can be imaged using dynamic contrast enhanced magnetic resonance imaging and tissue perfusion and extravasation can be measured.

In a fourth example, the effect of a hedgehog pathway inhibitor on the blood vessel density of a target tissue can be measured by fluorescence. For example, after administration of a hedgehog inhibitor to a mammal for a period of time, Lycospersicon esculentun lectin can be injected intravenously, followed by staining with CD31 antibodies (to visualize total vascular content of the tissue) on tissues harvested from the mammal. The stained tissue can be viewed using a confocal microscope to measure changes in tissue morphology, e.g., blood vessel perfusion, blood vessel patency, and blood vessel density.

In a fifth example, the effect of a hedgehog pathway inhibitor on stromal density in a tissue can be measured by harvesting and staining the tissues of interest. Accordingly, after administration of a hedgehog pathway inhibitor for a period of time to a mammal, a tissue sample is harvested and stained with one or more staining reagents, and viewed using confocal microscopy. Examples of staining reagents include, but are not limited to hematoxylin stain, eosin stain, Masson's trichrome stain, or Lillie's trichrome stain. Stained sections of tissue can be viewed under a confocal microscope at a magnification of about 20× to about 200×, or about 20× to about 100×, or about 20× to about 60×.

### Pharmaceutical Compositions

In certain embodiments wherein the hedgehog pathway inhibitor is administered with an agent (e.g., a therapeutic agent or an imaging agent), the hedgehog pathway inhibitor and the agent may be delivered in the same pharmaceutical composition or in different pharmaceutical compositions. In certain embodiments, the hedgehog pathway inhibitor and the agent are administered in the same pharmaceutical composition. In certain embodiments, the hedgehog pathway inhibitor and the agent are administered in different pharmaceutical compositions.

In certain embodiments, the hedgehog pathway inhibitor and the agent are administered by different routes (for example, one component is administered orally, while the other component is administered intravenously). In certain embodiments, the hedgehog pathway inhibitor and the agent are administered via the same route (e.g., both orally or both intravenously).

Pharmaceutical compositions may be formulated for administration in a solid or liquid form, such as those adapted for oral administration (for example, drenches, aqueous or non-aqueous solutions or suspensions, tablets, e.g., those targeted for buccal, sublingual, and systemic absorption, capsules, boluses, powders, granules, pastes for application to the tongue); parenteral administration (for example, by subcutaneous, intramuscular, intravenous or epidural injection such as, for example, a sterile solution or suspension, or sustained-release formulation); topical application (for example, as a cream, ointment, or a controlled-release patch or spray applied to the skin); intravaginally or intrarectally (for example, as a pessary, cream or foam); sublingually; ocularly; transdermally; pulmonarily, or nasally.

Pharmaceutical compositions may be formulated with one or more pharmaceutically acceptable carriers (additives) and/or diluents. Examples of suitable aqueous and nonaqueous carriers which may be employed in pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Pharmaceutical compositions may also contain adjuvants such as preservatives, wetting agents, emulsifying agents, dispersing agents, lubricants, and/or antioxidants. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like, into the compositions. In addition, prolonged absorption of the injectable pharmaceutical form may be brought about by the inclusion of additives which delay absorption, such as aluminum monostearate and gelatin.

Methods of preparing these formulations include the step of bringing into association one or more components of the pharmaceutical composition (i.e., the hedgehog pathway inhibitor and/or the agent), with the pharmaceutically acceptable carriers (additives), diluents and/or adjuvants. In general, the formulations can be prepared by uniformly and intimately bringing into association the one or more components with liquid carriers, or finely divided solid carriers, or both, and then, if necessary, shaping the product.

When the formulation is administered to mammals, it can be given per se or as a to pharmaceutical composition containing, for example, about 0.1 to 99%, about 10 to 50%, about 10 to 40%, about 10 to 30%, about 10 to 20%, or about 10 to 15%, of the one or more components in combination with a pharmaceutically acceptable carrier.

Actual dosage levels of the one or more components in the pharmaceutical compositions may be varied so as to obtain an amount of the component which is effective to achieve the desired therapeutic response for a particular mammal, composition, and mode of administration, without being toxic to the patient.

The selected dosage level will depend upon a variety of factors including the activity of the particular component employed, the route of administration, the time of administration, the rate of excretion or metabolism of the particular component being employed, the rate and extent of absorption, the duration of the treatment, other drugs, other compounds and/or materials used in combination with the particular component employed, the age, sex, weight, condition, general health and prior medical history of the mammal being treated, and like factors well known in the medical arts.

In general, a suitable daily dose of a component will be an amount which is the lowest dose effective to produce a therapeutic effect. Such an effective dose will generally depend upon the factors described above and herein.

When hedgehog inhibitors are administered in combination an agent (such as a chemotherapeutic agent or radiation) the daily dose of each component may be lower than the corresponding dose for single-agent therapy.

Doses of the components (e.g., the hedgehog pathway inhibitor and/or an agent) can range, for example, from about 0.0001 mg/kg to about 100 mg/kg, from about 0.001 mg/kg to about 100 mg/kg, from about 0.01 mg/kg to about 100 mg/kg, from about 0.1 mg/kg to about 100 mg/kg, from about 1 mg/kg to about 50 mg/kg, from about 0.0001 mg/kg to about 500 mg/kg, from about 0.001 mg/kg to about 500 mg/kg, from about 0.01 mg/kg to about 500 mg/kg, or from about 0.1 mg/kg to about 500 mg/kg.

The determination of the mode of administration and the correct dosage is well within the knowledge of the skilled clinician. For example, these doses can be administered daily, every other day, three times a week, twice a week, weekly, or bi-weekly. The dosing schedule can include a “drug holiday,” i.e., the composition can be administered for two weeks on, one week off, or three weeks on, one week off, or four weeks on, one week off, etc., or continuously, without a drug holiday. The compositions can be administered orally, intravenously, intraperitoneally, topically, transdermally, intramuscularly, subcutaneously, intranasally, sublingually, or by any other known route.

In certain embodiments, the hedgehog pathway inhibitor is administered at about or less than 100 mg/kg per day. In certain embodiments, the hedgehog pathway inhibitor is administered at about or less than 75 mg/kg per day. In certain embodiments, the hedgehog pathway inhibitor is administered at about or less than 50 mg/kg per day. In certain embodiments, the hedgehog pathway inhibitor is administered at about or less than 40 mg/kg per day. In certain embodiments, the hedgehog pathway inhibitor is administered at about 40 mg/kg per day.

In certain embodiments, the agent (e.g., a chemotherapeutic agent) is administered at about or less than 500 mg/kg per day. In certain embodiments, the agent is administered at about or less than 400 mg/kg per day. In certain embodiments, the agent is administered at about or less than 300 mg/kg per day. In certain embodiments, the agent is administered at about or less than 200 mg/kg. In certain embodiments, the agent is administered at about or less than 100 mg/kg per day. In certain embodiments, the agent is administered at about 100 mg/kg per day.

In certain embodiments, the hedgehog pathway inhibitor is administered at about or less than 100 mg/kg per day and the agent is administered at about or less than 500 mg/kg per day.

The invention now being generally described, it will be more readily understood by reference to the following examples, which are included merely for purposes of illustration of certain aspects and embodiments of the present invention, and are not intended to limit the invention.

### EXEMPLIFICATION

Pancreatic ductal adenocarcinoma (PDA) is profoundly insensitive to a broad variety of anti-neoplastic agents. Progress in understanding this feature of PDA has been limited by the absence of appropriate animal models. In contrast to traditional engraftment models, we found that an accurate mouse model of PDA was predominantly refractory to the chemotherapeutic gemcitabine. We implicated inefficient drug delivery as a mechanism of chemoresistance in this model and correlated this with decreased vascular density and poor intratumoral perfusion, features that are shared with human PDA. Intratumoral vascular density and gemcitabine delivery were increased upon treatment with the hedgehog pathway inhibitor, Compound A, correlating with transient disease stabilization and a significant extension of survival.

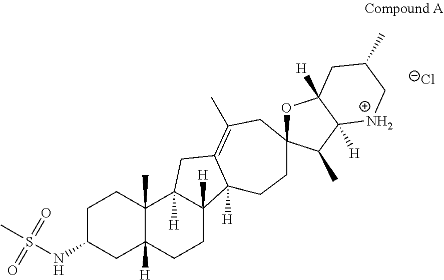

Pancreatic ductal adenocarcinoma is among the most intractable of human malignancies. Decades of effort have witnessed the failure of a multitude of chemotherapeutic regimens and the current standard-of-care therapy, gemcitabine (Gemzar, Eli Lilly), provides only a few weeks extension of survival. Currently, oncology drug development relies heavily on tumor transplantation models such as xenografts for efficacy testing of novel agents. However, existing models are typically quite responsive to numerous chemotherapeutic agents, including gemcitabine.

Genetically engineered mouse (GEM) models of cancer are an alternative to transplantation models for preclinical therapeutic evaluation. KPC mice are designed to conditionally express endogenous mutant Kras and p53 alleles in pancreatic cells, resulting in focal tumors that best mimic the pathophysiological and molecular aspects of the human disease in comparison to other GEM models of pancreatic cancer. We sought to evaluate the behavior of these tumors in response to therapy and further to investigate the mechanism of chemoresistance in this model.

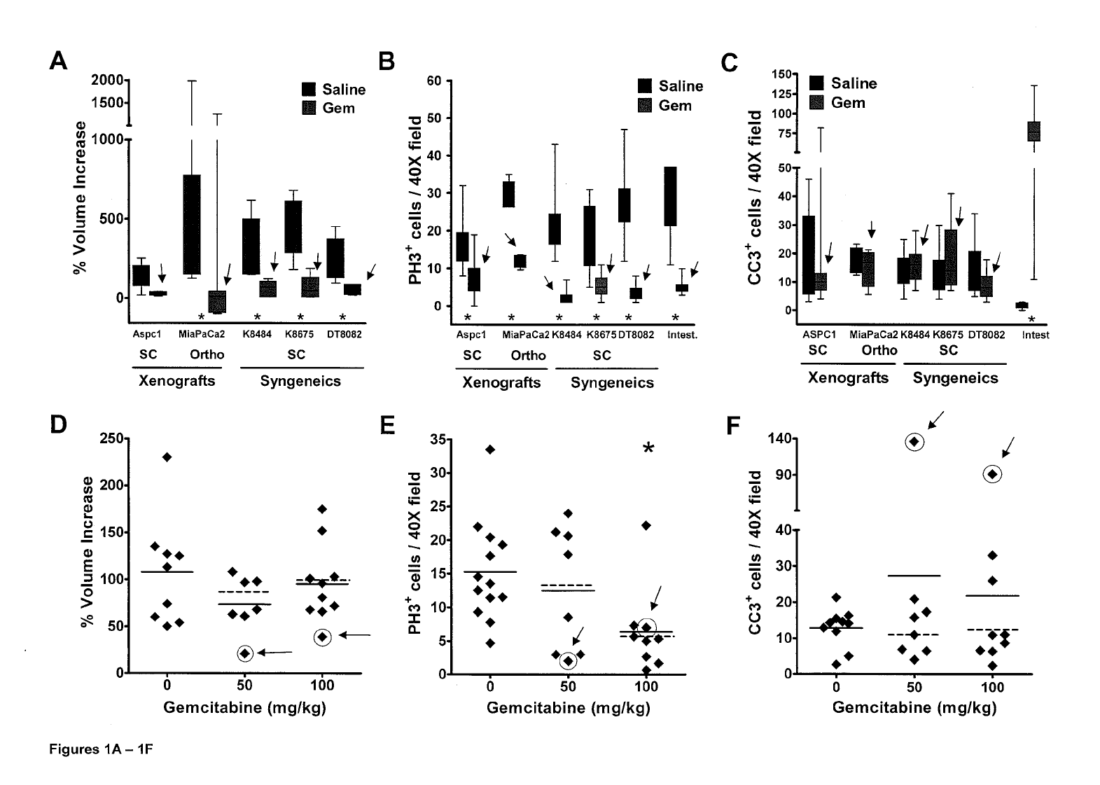

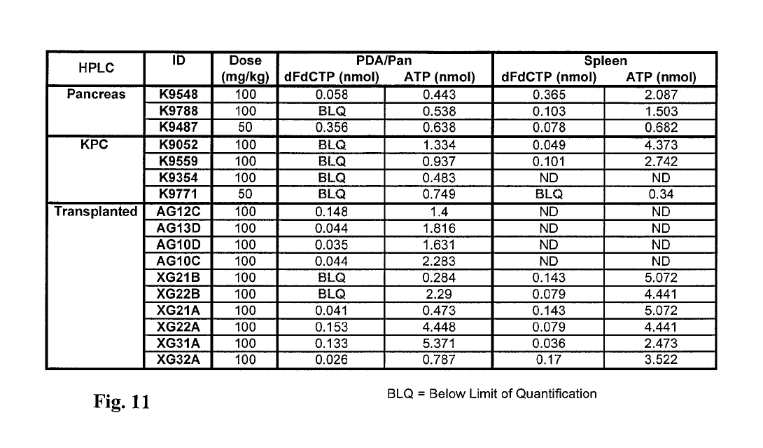

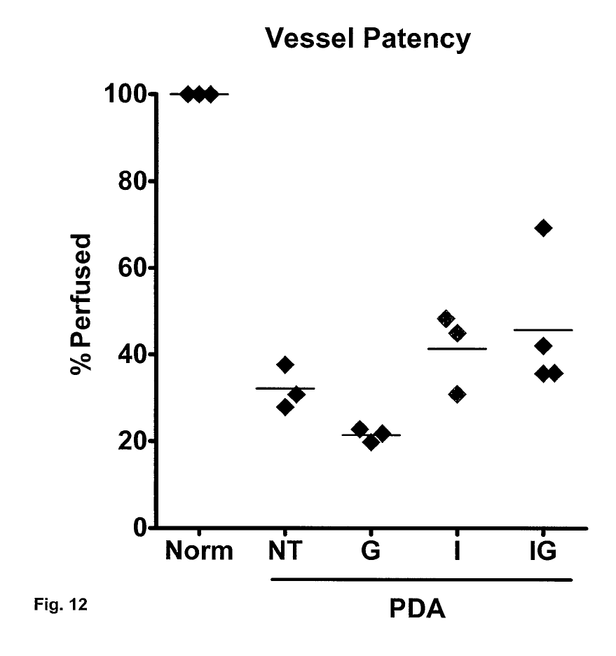

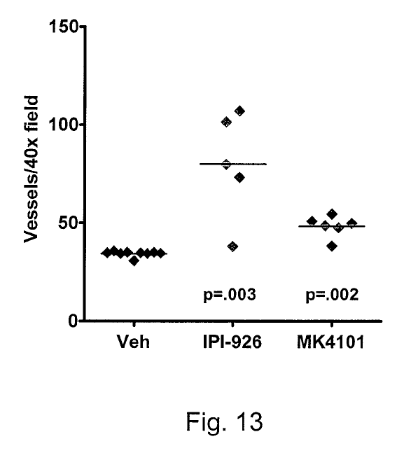

To assess the utility of GEM models of PDA in preclinical drug development, KPC mice harboring spontaneous pancreatic tumors were treated with gemcitabine and monitored by high resolution ultrasonography. The response of these tumors was compared to examples of three types of transplantation models (See Methods, FIGS. 1A-1F and FIG. 11). Gemcitabine treatment produced a substantial inhibition of tumor growth in all transplanted tumors, irrespective of human or mouse origin (FIG. 1A). In contrast, the growth of most KPC pancreatic tumors (N=15/17) appeared similar to that of control treated mice (FIG. 1D and FIG. 11). Although gemcitabine caused a reduction in the proliferative index in both transplanted and KPC tumors shortly after drug administration, this effect was less evident in KPC mice than in transplantation models and did not distinguish the two KPC mice that demonstrated an objective ultrasonographic response (FIGS. 1B and 1E). Rather, cellular apoptosis was substantially increased in these two tumors while remaining unaffected by treatment in other KPC or transplanted tumors (FIGS. 1C and 1F). Therefore, while transplanted tumor models are invariably sensitive to gemcitabine, the same treatment regimen does not influence tumor growth in the majority of KPC mice. This is consistent with the clinical activity observed with this agent wherein only 5-10% of patients demonstrate an objective radiographic response at the primary tumor site (Tempero et al., J. Clin. Oncol. (2003) 21:3402).

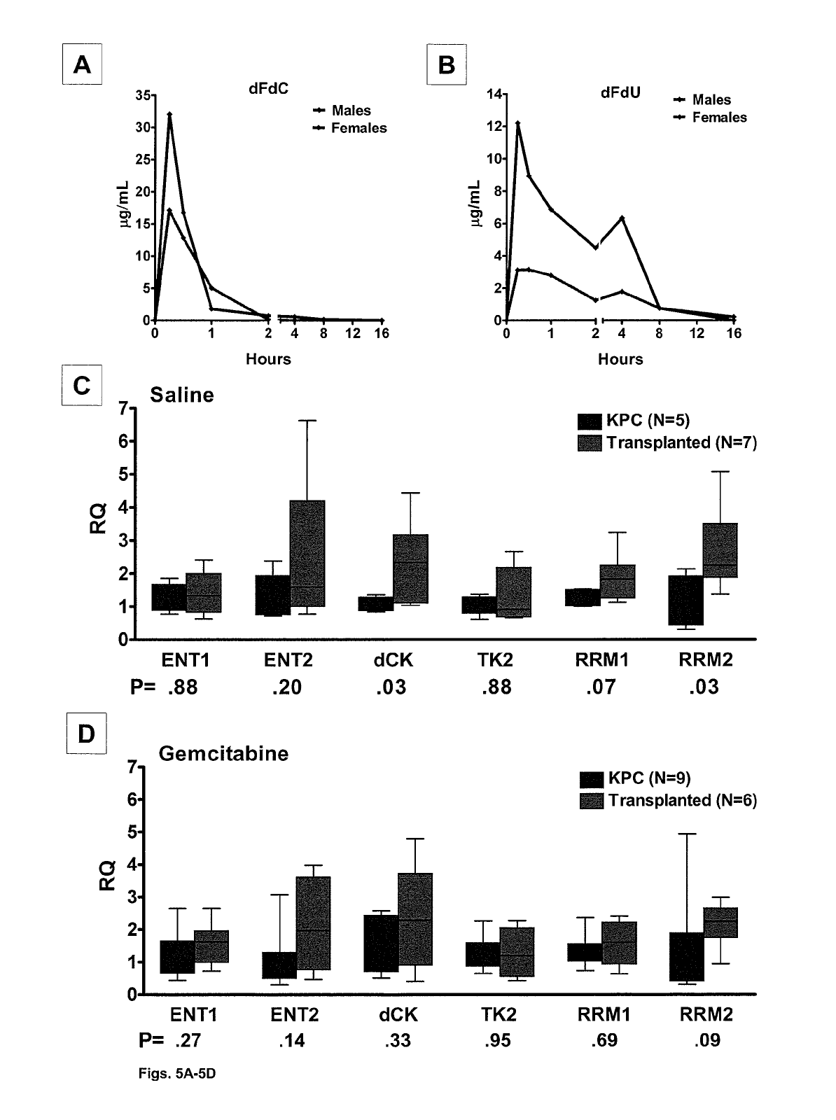

We noted that the transplantation of low-passage cells derived from KPC tumors yielded subcutaneous tumors that were nonetheless sensitive to gemcitabine treatment, suggesting that innate cellular differences did not explain their differential sensitivity. We therefore assessed pharmacological exposure to gemcitabine (2′,2′-difluorodeoxycytidine, dFdC) and its active, intracellular metabolite, gemcitabine triphosphate (2′,2′-difluorodeoxycytidine triphosphate, dFdCTP) by HPLC. Similar to the clinical studies, gemcitabine was rapidly deaminated to its inactive metabolite, difluorodeoxyuridine (dFdU) resulting in a short half-life for gemcitabine in circulating blood (FIGS. 5A and 5B). Using an approach developed for the assessment of gemcitabine metabolites in leukemia specimens, dFdCTP was present in transplanted tumor tissues and control tissues, but was absent in KPC tumors (FIG. 11). Therefore, dFdCTP accumulation in pancreatic tumor tissue distinguished transplantation and KPC models of PDA and correlated with the responsiveness of gemcitabine.

As gemcitabine sensitivity has previously been attributed to differences in the expression of equilibrative nucleotide transporter type 1 (hENT1), deoxycytidine kinase (dCK), and ribonucleotide reductase subunit M2 (RRM2), the expression of the murine homologues of these and related genes were investigated by real-time PCR in transplanted and KPC pancreatic tumors (FIGS. 5C and 5D). Increased expression was noted for dCK, the principal kinase responsible for mono-phosphorylation of dFdC, in transplanted tumors compared to KPC tumors (P=0.03, Mann-Whitney U). However, RRM2, a gene that promotes gemcitabine resistance, was also elevated in transplanted tumors (P=0.03, Mann-Whitney U). These trends were less apparent in tumors from mice treated with gemcitabine, indicating that gemcitabine resistance does not correlate with the expression of these genes (FIG. 5D).

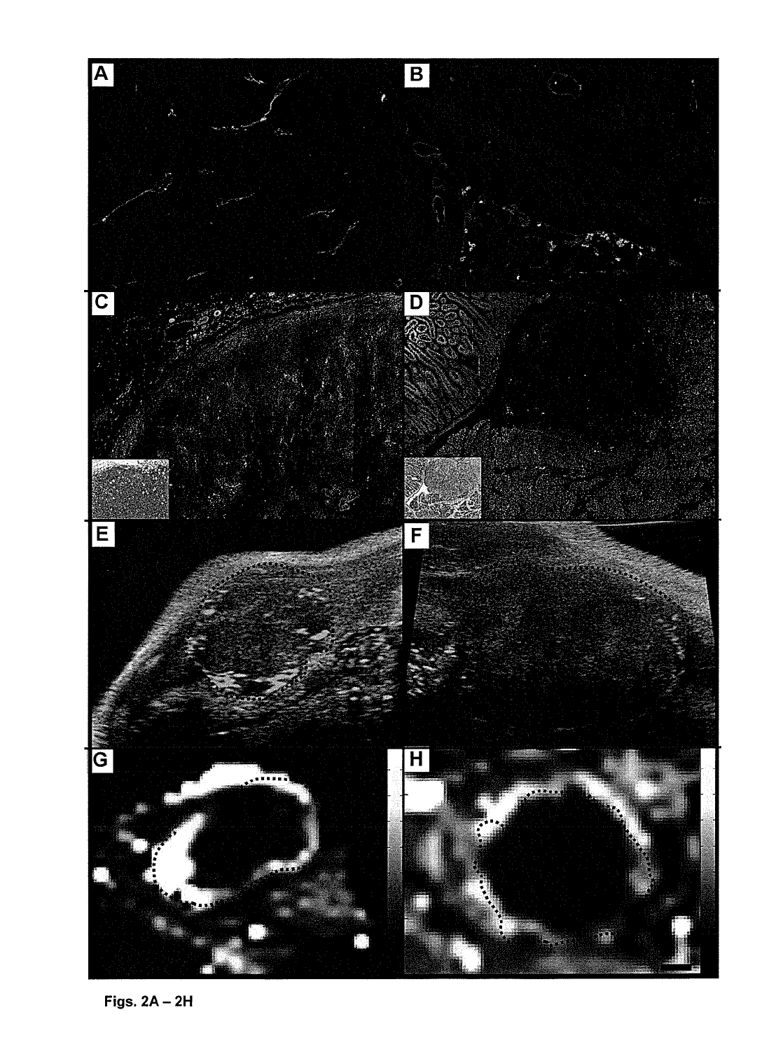

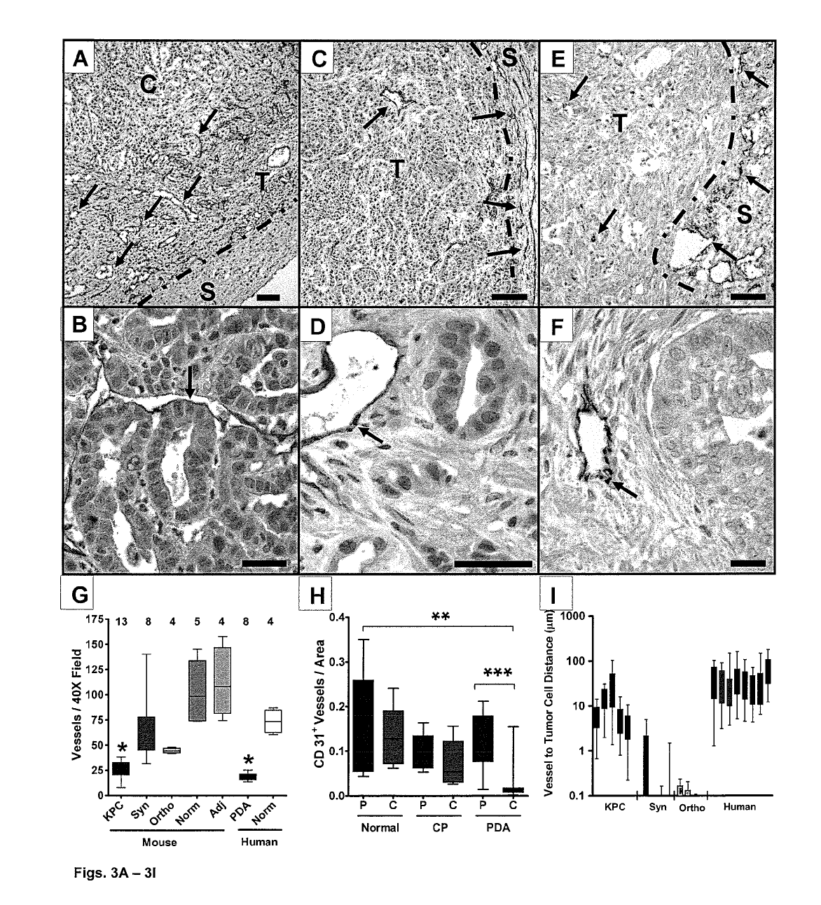

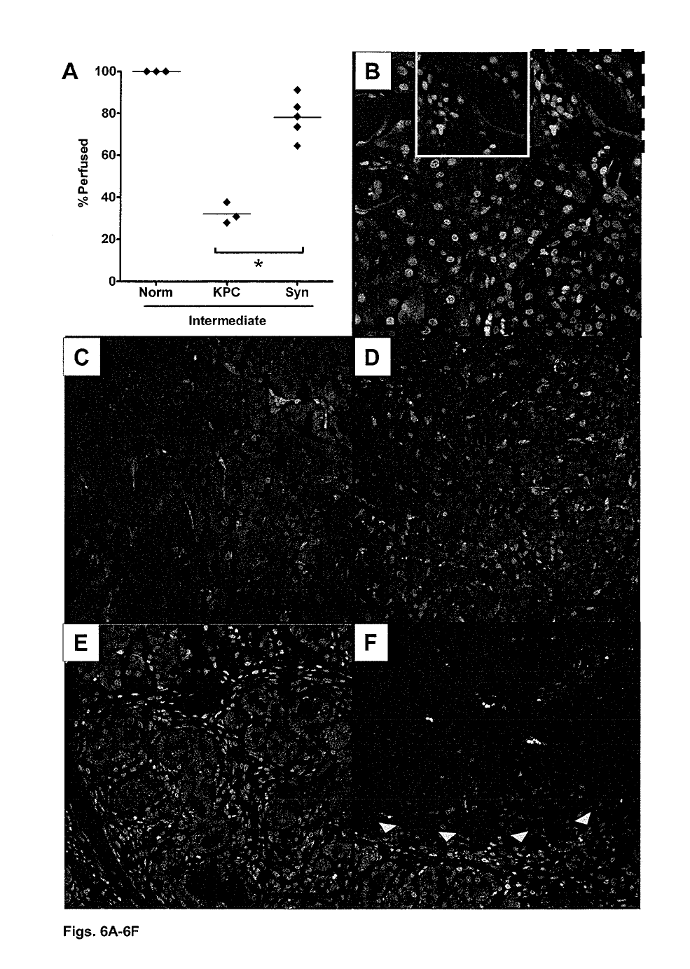

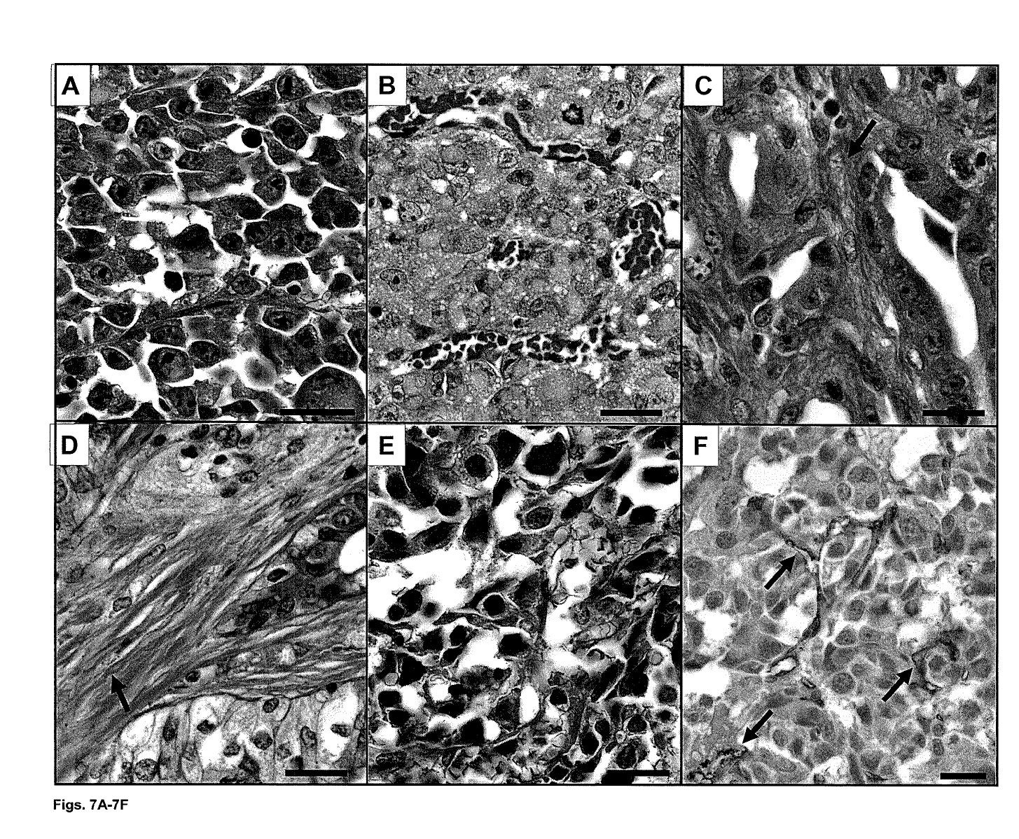

To investigate whether general abnormalities in drug delivery could contribute to the differential accumulation of gemcitabine in transplanted and KPC pancreatic tumors, the perfusion of tumor tissue was characterized in each model by multiple methods. First, functional vasculature was delineated in each model system by the intravenous infusion of Lycospersicon esculentum lectin in anesthetized animals, followed by the coimmunofluorescent detection of blood vessels in harvested tissues with CD31 antibodies. Using this approach, subcutaneous tumors demonstrated a patent vasculature, whereas KPC tumors had a poorly functional vasculature (FIGS. 2A and 2B). Indeed, only 32% of CD31⁺ blood vessels in KPC tumors were labeled with lectin, compared to 78% of transplanted tumors and uniform labeling in normal pancreatic tissues (FIG. 6A). Second, to evaluate whether small molecule intravascular delivery and drug penetration into tissues is generally impeded in KPC tumors, the fluorescent chemotherapeutic doxorubicin was co-administered intravenously with lectin (FIGS. 2C-2D and FIGS. 6B-6F). Confocal microscopy revealed a marked decrease in doxorubicin delivery to KPC pancreatic tumors in comparison to control tissues and transplanted tumors, confirming that both high molecular weight proteins and small chemicals were inefficiently delivered over a short time-course. Third, non-invasive imaging with contrast ultrasound was used to evaluate the vascular delivery of 5 μm microbubbles to tumors, clearly demonstrating the more efficient perfusion of transplanted tumors compared to KPC tumors (FIGS. 2E and 2F). Finally, the delivery of gadolinium-diethyltriaminepentaacetic acid (Gd-DTPA) was assessed by dynamic contrast enhanced magnetic resonance imaging (DCE-MRI, FIGS. 2G and 2H). We observed to significant enhancement in the periphery of transplanted tumors, while the cores exhibited a variable, heterogeneous pattern of enhancement consistent with large areas of necrosis observed by histology. In contrast, rapid and efficient delivery of Gd-DTPA was noted to the tissues surrounding KPC tumors, with little or no enhancement within the tumor body. Collectively, these four approaches demonstrated the poor delivery of small and large molecules to KPC pancreatic tumors in comparison to transplanted tumors, offering an explanation for the low accumulation of dFdCTP selectively in KPC tumors.

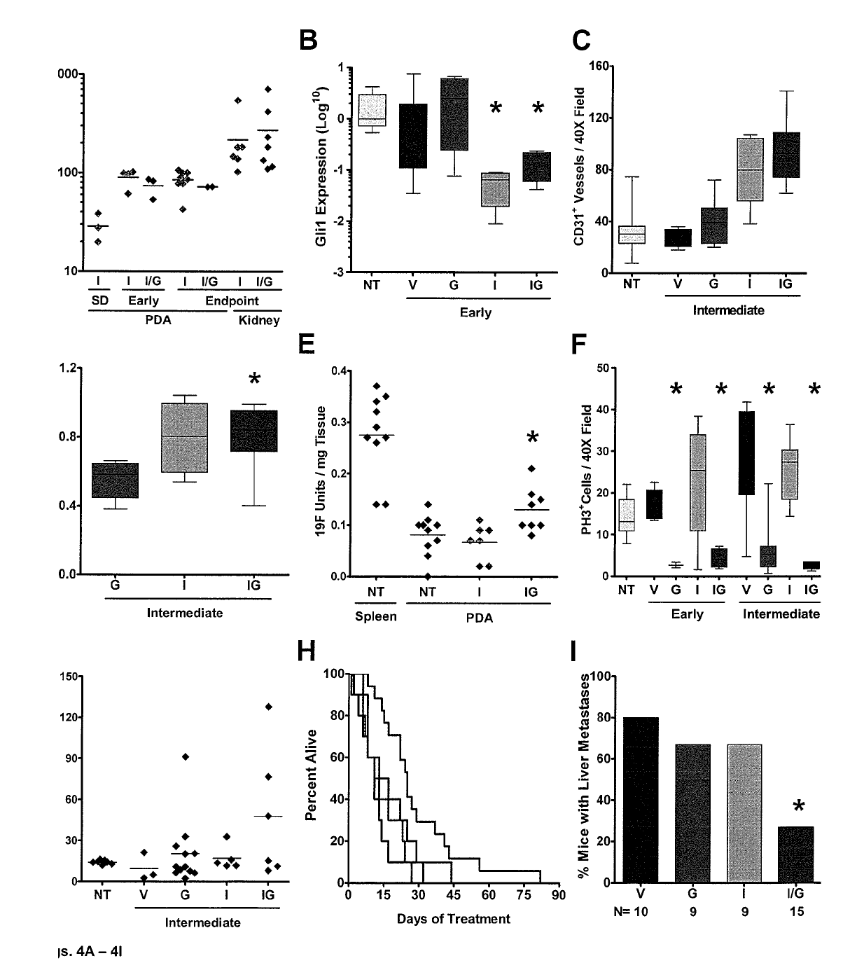

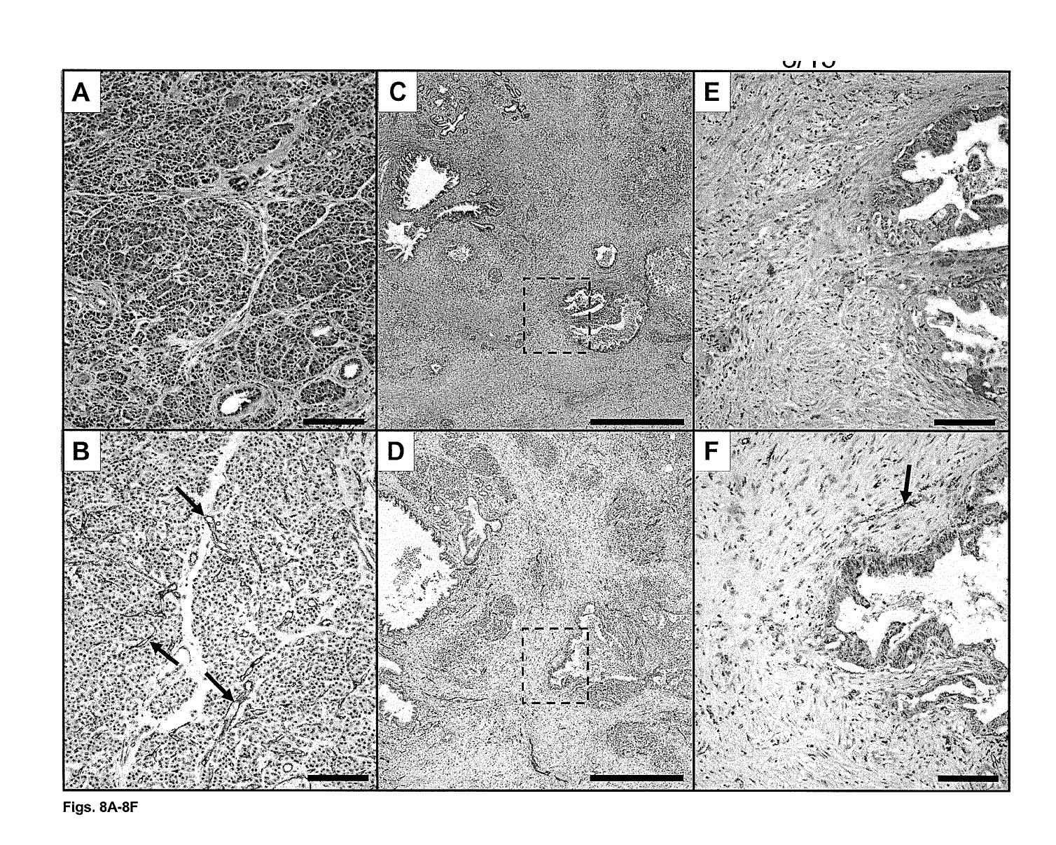

To investigate the etiology of poor tissue perfusion, the vascular content and tissue architecture was characterized in transplanted and KPC tumors. Consistent with our previous functional observations, transplanted tumors contained a dense zone of large vessels in the rim of the tumor, and a fine network of lacy vessels juxtaposed to neoplastic cells in the viable portions of tumor parenchyma (FIGS. 3A and 3B). In contrast, within the KPC tumor parenchyma, blood vessel density was markedly decreased; these vessels were often embedded within the prominent stromal matrix that is characteristic of these tumors and of primary human ductal pancreatic cancer (FIGS. 3C-3D and FIGS. 7A-7D). Most KPC tumors were surrounded by areas of preinvasive pancreatic cancer or inflammation that were densely vascularized with small and large vessels, consistent with observed enhancement surrounding KPC tumors by DCE-MRI and contrast ultrasound (FIGS. 2F and 2H). Interestingly, the Mean Vascular Density (MVD) was found to be much higher in both subcutaneous and orthotopic transplanted tumors in comparison to the invasive regions of KPC tumors (FIG. 3G), correlating with increased perfusion and gemcitabine delivery to transplanted tumors. To determine the clinical relevance of this result, we assessed the MVD in a collection of human primary PDA specimens and again found a greatly diminished vascular content in regions of overt carcinoma compared to adjacent normal pancreatic tissue (FIG. 3G, FIGS. 8A-8F). These results were extended to a larger, independent cohort of 18 human PDA specimens and compared to normal pancreatic tissues and chronic pancreatitis (CP) samples. To avoid the confounding effects caused by diffuse infiltration of adjacent pancreatic tissue, an image analysis algorithm was utilized to assess MVD in both peripheral and central regions of the samples (FIG. 3H). This approach confirmed that the core regions of pancreatic tumors are extremely hypovascular compared to CP and normal pancreas tissues (P<0.0001, Mann-Whitney U).

Finally, the distance separating intratumoral blood vessels from neoplastic cells in KPC and human pancreatic tumors was measured and compared to transplanted tumors (FIG. 3I). The average separation between the vessels and neoplastic cells in transplanted tumors was negligible, in contrast to a significantly increased distance in KPC (11.9 μm±4.6 SEM) and human (41.8 μm±4.6 SEM) pancreatic cancer samples. To extend these findings, we examined the two pancreatic tumors from KPC mice that demonstrated a cytotoxic and radiographic response. Indeed, these tumors contained very little stroma, a much higher MVD (57 and 171) and short distances between vessels and tumor cells (0.4 and 1.2 μm), compared to the averages for gemcitabine-resistant KPC tumors (FIGS. 7E and 7F).

Based on these observations, we reasoned that disrupting the stroma of pancreatic tumors might alter the vascular network and thereby enable more effective delivery of chemotherapeutic agents. Recently, Yauch et. al. demonstrated that the hedgehog (Hh) pathway participates in a paracrine signaling network in tumors and found that the genetic and pharmacological inhibition of this pathway specifically in stromal cells limited the growth of transplanted carcinoma cell lines (Yauch et al., Nature (2008) 455:406. Furthermore, the Hh pathway was shown to directly stimulate desmoplasia in a pancreatic transplantation model through the secretion of Sonic Hedgehog (Shh) ligand by neoplastic cells (Baily et al., Clin. Cancer Res. (2008) 14:5995. As Sonic Hedgehog (Shh) is overexpressed in the neoplastic cells of both human and KPC pancreatic tumors, we assessed the effects of Hh pathway inhibition on KPC tumors in combination with gemcitabine treatment.

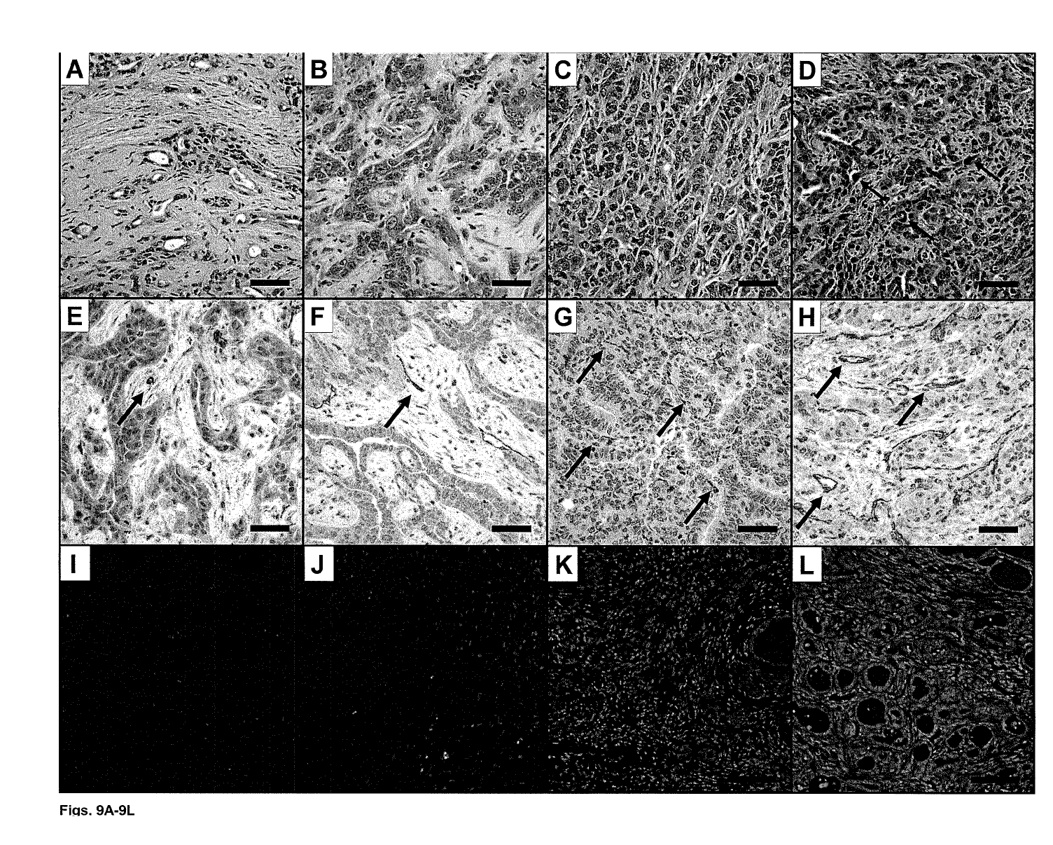

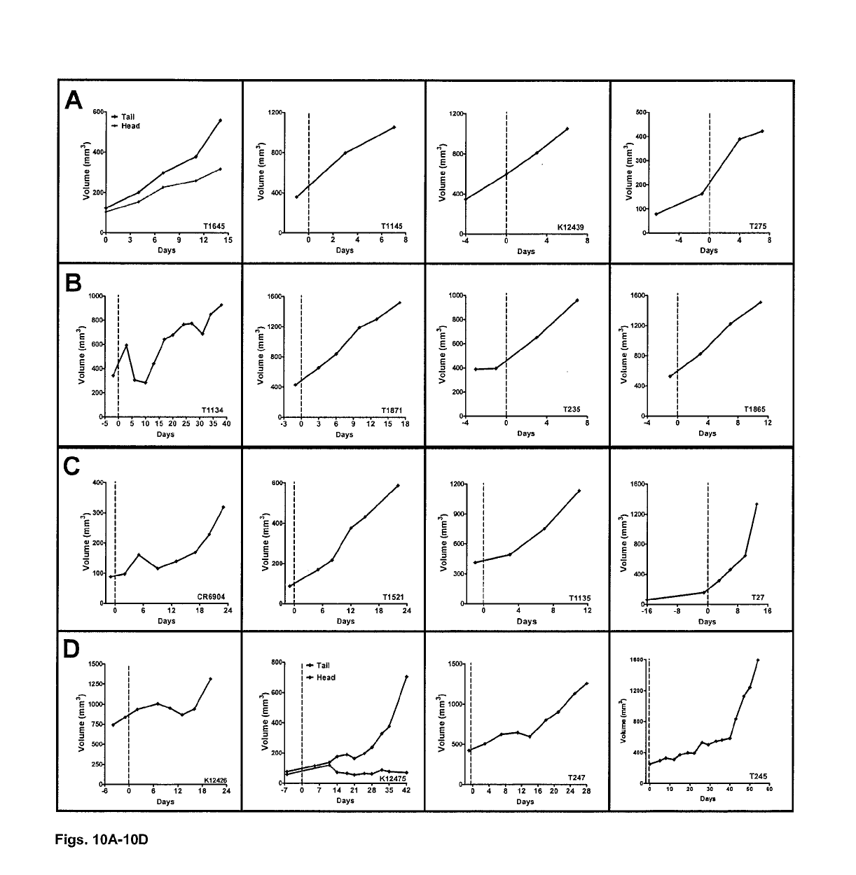

To inhibit the Hh pathway in KPC mice, we utilized the hedgehog pathway inhibitor Compound A. Oral administration of Compound A to KPC mice resulted in a measurable accumulation of drug in PDA tissues (FIG. 4A) and a significant decrease in the expression of Gli1, a transcriptional target of the Hh pathway (P<0.0001, Mann-Whitney U) (FIG. 4B). The effects on tumor architecture and perfusion were investigated in KPC mice after 8-12 days of treatment with Compound A or gemcitabine, alone or in combination (Compound A/gem). Vehicle and gemcitabine treated tumors harbored a dense stromal matrix with most tumors exhibiting a predominantly well-differentiated ductal epithelial phenotype (FIGS. 9A and 9B). In contrast, tumors treated with Compound A alone and those treated with Compound A/gem to exhibited dramatically altered histological patterns (FIGS. 9C and 9D). In particular, Compound A/gem treated tumors appeared depleted of desmoplastic stroma, resulting in densely packed ductal tumor cells. Regions of extreme nuclear and cellular atypia were commonly noted, lending to a more anaplastic appearance, particularly those treated with Compound A/gem. Finally, large areas of cavitating necrosis were frequently observed in tumors from Compound A/gem treated mice, indicative of a substantial therapeutic response.

Compound A also had a profound effect on the tumor vasculature, with a higher MVD noted in the tumors from Compound A treated mice (FIG. 4C). This effect was even more significant in Compound A/gem treated mice, where the MVD approximated that of normal pancreatic tissue. Furthermore, the intratumoral blood vessels from Compound A and Compound A/gem treated mice were positioned in close proximity to tumor cells in comparison to control and gemcitabine-treated specimens (FIGS. 9E-9H). Finally we determined that the increased MVD observed in Compound A treated mice correlated with more effective delivery of doxorubicin to tumor tissues. In particular, doxorubicin delivery to Compound A/gem treated tumors was significantly elevated over gemcitabine-only treated tumors, and this trend was evident but more variable in mice treated only with Compound A (FIG. 4D and FIGS. 9I-9L).

Given the improved delivery of doxorubicin after Compound A treatment, we sought to determine whether gemcitabine delivery was similarly improved. We developed a ¹⁹F NMR technique to measure all fluorinated metabolites derived from gemcitabine in tissue and found that the gemcitabine metabolite content in KPC tumors was only about one third that of other normal tissues (FIG. 4E). Treatment of KPC mice with Compound A/gem for 10 days resulted in a 60% increase in gemcitabine delivery compared to untreated tumors (FIG. 4E, P=0.04, Mann-Whitney U). No difference was observed in tumors treated only with Compound A perhaps reflecting the increased vasculature in Compound A/gem treated mice as compared to Compound A alone.

Having established that Compound A facilitates the delivery of gemcitabine to PDA, we investigated the effects of Compound A/gem on proliferation and apoptosis after 8-12 days of treatment. Similar to gemcitabine alone, Compound A/gem treatment produced a significant decrease in proliferation (FIG. 4F). In contrast, Compound A alone had little appreciable effect on cellular proliferation, consistent with the recent findings that human PDA cells lack primary cilia (the site of Smo activity) (Seeley et al., Cancer Res (2009) 69:422) and that conditional Smo deletion in pancreatic cells does not alter the progression of mutant Kras-induced preinvasive and invasive PDA (Nolan-Stevaux et al., Genes Dev (2009) 23:24). Interestingly, half of the tumors treated with Compound A/gem had elevated levels of Cleaved Caspase 3. While the average amount of apoptosis was not statistically significant between the cohorts (14.2 vs. 48.0, P=0.17) the trend may reflect a degree of heterogeneity in the timing of onset of apoptosis since some of the tumors with low apoptosis had substantial indications of necrosis by histology.

Finally, we treated cohorts of tumor-bearing KPC mice and monitored survival. Neither gemcitabine nor Compound A treatment had an effect on the survival of KPC mice. In contrast, combination treatment with Compound A/gem significantly extended the median survival of KPC mice by more than two-fold (P=0.001, Log Rank Test), yielding a hazard ratio of 6.36. (FIG. 4H). Furthermore, Compound A/gem treatment resulted in a significant decrease in metastases to the liver (FIG. 4I, P=0.015, Fisher's Exact). Analysis of individual tumor volumes found that most Compound A/gem-treated tumors exhibited a transient decrease in size after one to two weeks of treatment (FIGS. 10A-10C). In contrast, only a minority of gemcitabine (2/10) and Compound A (2/10) treated mice demonstrated objective ultrasonographic responses to treatment. In summary, Compound A/gem treatment of KPC mice increases the delivery of gemcitabine to PDA tumor tissue, significantly prolongs lifespan and decreases metastatic burden.

### Methods

A number of different types of mouse models are described herein. For clarity, the terms for these models have been defined below.

Genetically Engineered Mouse (GEM)—Model based on manipulation of the mouse genome, either through transgenic incorporation of exogenous DNA elements or following homologous recombination in embryonic stem cells.

transplantation model—refers to all mouse models in which tumor cells or tumor fragments are transplanted into a mouse.

xenograft—refers to models in which human tumor cells or tumor fragments are transplanted into immunodeficient mice.

syngeneic autograft—refers to models in which murine tumor cells or tumor fragments are transplanted into histocompatible, immune-competent mice.

ectopic—term that describes the site of transplantation as being different than that from which the transplanted material was derived.

subcutaneous (SC)—describes the location of an ectopic transplant as being under the skin.

orthotopic—term that describes the site of transplantation as being analogous to that from which the transplanted material was derived, in this case the pancreas.

(i) Statistical Analyses

Statistical analyses were carried out using GraphPad Prism version 5.00 for Windows, GraphPad Software, San Diego Calif. USA. Distinction of responders by Cleaved Caspase 3 was determined using Extreme Studentized Deviate outlier analysis. Significance of metastasis data was determined by Fischer's Exact test. All other comparisons were made using Mann-Whitney U test. Box plots show range, median and quartiles.

(ii) Cell Lines

The human pancreatic cancer cell line AsPc1 was cultured according to instructions. Mouse pancreatic cancer cell lines K8484, K8675 and DT8082 were isolated from tumors arising in KPC mice using a modification of the protocol described by Schreiber et al., Gastroenterology (20004) 127:250. Briefly, a 3 mm³ fragment of PDA was excised, washed in 10 mL of cold PBS, and then finely diced with sterile razors. Cells were incubated in 10 mL of collagenase solution at 37° C. for 30-45 minutes with mixing (1 mg/mL collagenase V in DMEM/F12). Cells were spun (100 rpm, 5 min.) and resuspended in 0.05% Trypsin/EDTA for 5 min. at 37° C., and then quenched with DMEM supplemented with 10% fetal calf serum and 96 μM CaCl₂. Cells were washed 3 times with DMEM/F12 medium and plated in a 6-well Biocoat dish (Becton Dickenson) in the ductal cell medium. After 3-4 passages, cells were transferred to standard plastic tissue culture dishes and grown in DMEM+10% FCS.

(iii) Subcutaneous and Orthotopic Tumor Models

1×10⁶ cells suspended in 100 μL of PBS were injected subcutaneously into the flanks of nude mice (xenografts) or into immunocompetent mice (syngeneic). For syngeneic models, recipient mice were descended from mice used to generate the KPC PDA cell lines. Orthotopic tumors with MiaPaca2 were generated as previously described (Niedergethmann et al., Br J Cancer 97, 1432 (Nov. 19, 2007)). Long (L) and short (S) axes of each tumor were measured with calipers (for subcutaneous tumors) or ultrasound (for orthotopic tumors). Tumor volume (V) was calculated: V=(L×S²)/2. Tumor volumes were normalized relative to the volume at the start of drug treatment for subcutaneous tumors. Orthotopic tumors were measured on days 7 and 20 following injection of cells and gemcitabine treatment was initiated on day 8. Tumor images were acquired using a pediatric ultrasound machine. This machine was not equipped for 3D reconstruction, so the same formula V=(L×S²)/2 was used to estimate the tumor volumes and changes. Mice were treated with gemcitabine by intraperitoneal injection on a Q3Dx4 schedule. When appropriate, a fifth dose was given on the final day four hours prior to necropsy for pharmacological analyses.

(iv) KPC Mice

KPC mice harbor heterozygous conditional mutant alleles of Kras and p53 as well as a pancreatic-specific Cre recombinase, Pdx1-Cre. Mice bearing the Kras, p53 and Cre alleles develop a full spectrum of premalignant neoplasms that stochastically undergo loss of the remaining wild-type Trp53 allele and culminate in overt invasive and metastatic PDA with a mean survival of 4.5 months. The KPC mice utilized in this paper harbor one of two conditional point-mutant p53 alleles: p53^{LSL-R172H} or p53^{LSL-R270H}. Kras^{LSL-G12D/+}, p53^{R172H/+}, Pdx1-Cre mice have been reported previously, but compound mutant mice with the latter allele, Kras^{LSL-G12D/+}, Trp53^{LSL-R270H/+}, Pdx1-Cre, have not been previously reported. These mice develop advanced pancreatic ductal adenocarcinoma that appears similar to mice harboring the Trp53^{R172H} allele.

(v) Drug Preparation

Gemcitabine (Gemzar™, Eli Lilly) powder (a ˜48% preparation of difluoro-deoxycytidine, dFdC) was purchased (Hannas, Delaware) and resuspended in sterile normal saline at 5 mg/mL dFdC. Additional Gemzar solution was provided by Addenbrooke's Hospital Pharmacy in Cambridge, UK and diluted with normal saline to 5 mg/mL dFdC. Drug was administered by intraperitoneal injections at the indicated dose.

Compound A was dissolved in a 5% aqueous solution of Hydroxypropyl-β-cyclodextran (HPBCD) to a concentration of 5 mg/mL (accounting for batch potency), with sonication and vortexing, and then sterile filtered through a 0.22 μM Millex GV syringe filter. Working solution was stored at 4° C. for up to one week.

(vi) Drug Study Treatment Groups

For FIGS. 1-3, mice were treated with either saline (20 μL/g of 0.85% NaCl) or saline containing 50 or 100 mg/kg of gemcitabine. For FIG. 4, the following four treatment groups were described at various timepoints:

vehicles: 20 μL/g 0.85% NaCl+8 μL/g 5% HPBCD;

gemcitabine: 100 mg/kg gemcitabine+8 μL/g 5% HPBCD;

Compound A: 40 mg/kg Compound A+20 μL/g 0.85% NaCl;

Compound A/gem: 40 mg/kg Compound A+100 mg/kg gemcitabine.

For survival studies, mice were enrolled following the detection of 5-10 mm diameter tumor by ultrasound. Tumors were quantified by 3D ultrasound twice weekly until endpoint.

(vii) Imaging and Quantification of KPC Pancreatic Tumors

High resolution ultrasound (US) imaging of normal and diseased mouse pancreas using the Vevo 770 System with a 35 MHz RMV scanhead (Visual Sonics, Inc.). 3D images were produced using the 3D motor arm to collect serial images at 0.25 mm intervals through the thickness of the tumor. Tumors were outlined on each 2D image and reconstructed to quantify the 3D volume using the integrated Vevo 770 software package.

(viii) Contrast Ultrasound

Mice were imaged by ultrasound as described previously (Cook et al., Methods Enzymol. (2008) 439:73). Baseline images were acquired in Contrast Mode and then an 80 μL bolus of unconjugated Vivo Micromarker suspension (VisualSonics, Inc.) was administered via tail vein catheter during acquisition of a second contrast video. The baseline image was subtracted from the contrast image and the difference was displayed with a contrast setting of 80 and a threshold setting of 0.

(ix) MRI

Magnetic resonance imaging experiments were carried out on a Varian MRI system (Varian, Inc, Palo Alto, Calif., USA) equipped with a 9.4T horizontal bore cryo-cooled superconducting magnet of 210 mm bore and a gradient set of strength 40 G/cm, 120 μsec risetime and internal diameter 120 mm. The imaging probe used was a Varian Millipede of 40 mm internal diameter. Mice were anaesthetized with Hypnorm/Hypnovel. Anatomical images were obtained using coronal T2-weighted fast spin-echo (repetition time TR=2000 ms, effective echo time TE=25 ms, echo train length 8 echoes, 512×512 points, field of view 80×80 mm, slice thickness/gap 1.5/0.5 mm, 12-15 slices) with chemical shift-selective fat suppression and respiratory gating. All other images were matched to the slice positions and field of view of the anatomical images. Baseline T1 maps were obtained from either T1-weighted RF-spoiled gradient echo imaging (TE=1.52 ms, TR=0.05/0.1/0.2/0.5/1/2/5 seconds, 128×128 points, α=60°) or inversion recovery turbo-FLASH (TE=1.52 ms, TR=3 ms, inversion time TI=0.2/0.5/1/2/5/10 seconds, 128×128 points) using a non-slice-selective hyperbolic secant adiabatic inversion pulse. The dynamic contrast-enhanced (DCE-MRI) time course was acquired using T1-weighted RF-spoiled gradient echo (TE=1.52 ms, TR=50 ms, 128×128 points, α=45°); 5 time points were acquired before injection of 0.1 mmol/kg Gd-DTPA (Magnevist, Bayer Schering Pharma AG) and 100 after, giving a total imaging time of 10 minutes. Additionally, high-resolution (256×256 points) T1-weighted images were acquired before contrast administration and after the DCE-MRI time course, and flip angle mapping data acquired to correct for coil radiofrequency inhomogeneity. DCE-MRI data were analyzed in software custom-written in MATLAB 7.4 (The Mathworks, Inc, Natick, Mass., USA) using the model of Tofts and Kermode to evaluate the pharmacokinetic parameters Ktrans and ve, and additionally calculating the area under the [gadolinium]-time curve over the first 60 seconds post-injection (IAUGC60), as recommended by a panel of experts for vascular-related studies in oncology.

(x) ¹⁹-Fluorine Nuclear Magnetic Resonance

Mice were treated with indicated regiments for 12 days. On the final day, mice from all cohorts were administered an i.p. injection of 100 mg/kg gemcitabine and sacrificed after one hour. Tissues were rapidly dissected and snap frozen in liquid nitrogen. Tissue specimens were maintained at −80° C. until subjected to nucleotide extraction. Samples were homogenized in a Qiagen TissueLyser with a 5 mm ball bearing for 2×6 minutes at 25 KHz, in the presence of 4 volumes of ice-cold acetonitrile (μL acetonitrile=4×μg of tissue). An equal volume of water was added and then the sample was spun at 14,000 rpm for 10 minutes at 4° C. Supernatant was freeze dried, resuspended in 600 μL of D₂O and transferred to a 5 mm standard NMR tube (Wilmad) for ¹⁹F NMR analysis on a Bruker 600 MHZ (14.1T) Avance NMR spectrometer using a QNP probe. Acquisition parameters included a 1D pulse sequence of ¹⁹F observation and inverse-gated ¹H decoupling, spectral sweep width of 177 ppm (100000 Hz), 4096 scans and 1.65 sec of repetition time. Total acquisition time for each sample was about 1 hr 55 min. Trichloro-fluoro-methane was used for ¹⁹F NMR chemical shift calibrations. A broad hump observed in the baseline of ¹⁹F NMR spectra was removed by application of Linear-prediction (LP) back projection to the time domain data by using 2000 (number of LP) coefficients and 128 back-prediction points prior to Fourier transformation and phase correction. All fluorine peaks were integrated using the Bruker Topspin software processing package to provide a measure of total gemcitabine metabolite concentration in the sample. These integrated values were normalized to the tissue weights and presented in arbitrary units. In pilot experiments, we found that six hours after gemcitabine dosage, all ¹⁹F signals were absent from PDA and control tissues.

(xi) Pharmacology

KPC mice harboring pancreatic tumors were treated with 40 mg/kg of Compound A by oral gavage, either singly, once daily for four days or at as part of a survival study. In addition, mice received twice weekly injections of either 100 mg/kg gemcitabine or 20 μL/g saline, as indicated. Six hours following the final dose, tissue samples were harvested, snap frozen in liquid nitrogen and stored at −80° C. Mice with abdominal ascites were excluded from analysis. Samples were analyzed by LC/MS, as described below.

(xii) Analytical Methods

Calibration standard stock solutions were prepared by dissolving Compound A at a concentration of 2.5 mg/mL in DMSO. Internal standard stock solution was prepared by dissolving deuterated Compound A (Compound A-d³) in DMSO for a final concentration of 2.5 mg/mL. Stock solutions were stored in aliquots at −80° C. until further use.

Water (HPLC grade) was obtained from Mallinckrodt Chemicals (Phillipsburg, N.J.). Acetonitrile (HPLC grade) was purchased from JT Baker (Phillipsburg, N.J.) and formic acid was supplied by Fluka Chemie (Buchs, Switzerland). DMSO was purchased from Sigma (St. Louis, Mo.). Powdered phosphate buffered saline was reconstituted with water to a 0.1 M concentration (pH 7.2)

Calibration standard and internal standard stock were thawed at room temperature. Internal standard solution was made by diluting deuterated Compound A into 10% MeOH solution for a final concentration of 25 ng/mL. Calibration curves were prepared in ACN:PBS homogenization buffer and diluted into internal standard solution. The assay had a final LLOQ of 0.78 ng/mL. In addition, ACN:PBS with and without internal standard (QC0 and blank, respectively) were included in the analytical run.

Tumor samples were homogenized in 4 volumes of ACN:PBS buffer. Pre-weighed tissue samples were added to 5 mL polycarbonate tubes (SPEX CertiPrep part number 2241-PC) containing a single steel milling ball (SPEX CertiPrep part number 2156) and were homogenized using a Geno/Grinder from SPEX CertiPrep (Metuchen, N.J.) for 2 minutes. Homogenates were then filtered using a 0.45 μm low binding hydrophilic multiscreen solvinert plate (Millipore, part number MSRLN0410) and collected in a 96-well receiving plate. The tissue filtrates were then diluted 1:1 (equal volume) and 1/100 into internal standard solution. Compound A concentrations for all tissues were preferentially determined using the 1:1 dilution unless any of the replicates for a given tissue required the higher dilution of 1/100 for accurate quantitation.

Compound A concentrations in the samples were determined from the calibration curves generated in homogenization buffer. A dilution factor of 4 was applied to the tissue samples to account for the volume of buffer added to each tissue for homogenization. When adjusting for dilution factor, the assay LLOQ is 3.1 ng/g. No correction for extraction efficiency was applied.

Sample analysis was performed on an Agilent 1200 from Agilent Technologies (Santa Clara, Calif.) coupled with an API-4000 mass spectrometer from Applied Biosystems (Foster City, Calif.) for detection of Compound A and Jervine by multiple reaction monitoring (MRM).

Twenty μL of the samples were injected on an analytical column (Symmetry IS, C18, 2.1×20 mm, 3.5 μm, from Waters, Milford, Mass.) and eluted with a 4 minute gradient from 5-95% acetonitrile in H₂O, 0.1% (v/v) formic acid. Mass spectrometric detection of Compound A and Compound A-d³ was performed by MRM with the following transitions for each compound:

TABLE 6

| Compound | Q1 mass (m/z) | Q3 mass (m/z) |
|---|---|---|
| Compound A | 505.4 | 114.1 |
| Compound A-d³ | 508.3 | 84.1 |

The data were acquired and processed using the software Analyst 1.4.1 (Applied Biosystems, Foster City, Calif.). For the standard curve samples, peak area ratios of Compound A to internal standard (jervine) were calculated and plotted against the theoretical concentrations. A weighing factor of 1/x² was applied to the data. Sample concentrations, as measured by their peak area ratios (analyte divided by internal standard), were determined from the calibration curves.

(xiii) Determination of dFdCTP Concentration by HPLC

Mice were injected i.p. with 50 or 100 mg/kg gemcitabine and sacrificed after four hours. Tissues were rapidly dissected and snap frozen in liquid nitrogen. Specimens were maintained at −80° C. until nucleotide extraction. Specimens were ground under liquid nitrogen with a mortar and pestle. The powdered contents were suspended in 0.4N perchloric acid and sonicated in an ice bath. Solids were removed by centrifugation, the pellet was washed with perchloric acid, and the supernatants were combined. Following neutralization with KOH and removal of KClO₄ by centrifugation, a portion of the supernatant was analyzed by high-pressure liquid chromatography. The amount of gemcitabine triphosphate was normalized to the ATP level determined in the same sample analysis. Samples with inadequate concentrations of ATP were excluded from analysis.

(xiv) Immunohistochemistry

Tissues were fixed in 10% formalin solution for 24 hours and transferred to 70% ethanol. Tissues were paraffin embedded, sectioned and rehydrated. For CD31 IHC, sections were unmasked in 10 mM EDTA, pH 8.0 in a pressure cooker. For all other antibodies, sections were unmasked in 10 mM citric acid in a pressure cooker. Endogenous peroxidases were quenched in 3% H₂O₂/PBS for 20 minutes. Remaining steps were carried out with Vectastain ABC kits appropriate to the species of primary antibody (Vector Labs, Burlingame, Calif.) with the following modification: blocking serum was supplemented with Protein Blocking Agent (Immunotech/BeckmanCoulter, Fullerton, Calif.) diluted 1:50. Antigens were developed with DAB Peroxidase Substrate (Vector Labs). The following antibody dilutions were used: Phospho-Histone H3, 1:100 (#9701, Cell Signaling Technology); Cleaved Caspase 3, 1:100 (#9661, Cell Signaling Technology); CD31, 1:75 (SC-1506). Slides were counterstained with hematoxylin.

(xv) Mean Vascular Density and Vascular-Tumor Distance

Tissue sections were probed with anti-CD31 antibodies and counterstained with hematoxylin. Mean vascular density (MVD) was determined as the number of CD31 positive blood vessels per 40× field, over three random fields per tumor. The distance separating intratumoral blood vessels and neoplastic cells was determined for twenty randomly chosen blood vessels per tumor. Each intratumoral blood vessels was photographed at 1000× magnification, and the distance to the four nearest tumor cells was measured and the results averaged.

(xvi) Computer Aided Image Analysis of Mean Vessel Density in Human Tissues

Frozen sections (10 um thickness) were prepared from histologically confirmed samples of infiltrating colon cancer, infiltrating pancreatic cancer or chronic pancreatitis from resection specimens and fixed in 4% paraformaldehyde for 10 minutes at 4° C. Sections were then washed in 1×TBS three times followed by incubation with blocking serum (1×TBS/5% BSA/0.04% Triton X100) for 3 hours at 4° C. Slides were washed with 1×TBS, then incubated overnight at 4° C. with primary antibodies diluted in blocking serum (1:200 dilution phycoerythrin labeled mouse anti-CD31 (#340297), Becton Dickonson Systems and 1:100 dilution rabbit anti-TEM8 (H-140), Santa Cruz Biotechnology). After washing in 1×TBS, sections were incubated with 4′,6-diamidino-2-phenylindole, dihydrochloride (DAPI). Slides were coverslipped and labeling visualized using a Nikon E800 microscope.

Immunolabeled slides were scanned at 20×, and up to five 1500×1500 pixel or 735 μm2 fields located both centrally and at the periphery of the pancreas were extracted and analyzed by color deconvolution (ImageJ software). Thresholding was used to convert the image to a binary format in which lighter background staining was eliminated and the remaining areas of staining were converted into particles, which could be individually analyzed by the software. Accounting for variations in staining intensities among slides, exclusion of background staining was based on the average intensity of the overall staining. Any particles measuring less than 150 pixels (73.5 μm²) were excluded to reduce the degree of large vessel fragmentation and the presence of single immunoreactive cells. To determine the immunoreactive tissue area, the area of the slide without immunoreactivity was subtracted from the overall area of the field. The microvessel density per tissue section was calculated by determining the average ratio of vessel area to the total tissue in five fields per tissue section.

(xvii) Vascular Labeling and Drug Diffusion

To assess the functional vasculature, biotin-conjugated Lycopersicon esculentum lectin (B1175-1 mg, Vector Laboratories) was resuspended in 425 μL PBS and mixed with 75 μL of 1 mg/mL Streptavidin-AlexaFluor 633 (S21375, Molecular Probes) (in sterile PBS). Prior to use, the lectin-avidin mixture was centrifuged 14,000 k on a microfuge for 10 minutes to remove any particulate. Fifteen minutes prior to euthanasia, 100 μl (0.4 mg total) of the conjugated lectin was administered as a slow intravenous infusion over 5 minutes, and the hemodynamics monitored closely to ensure that it was tolerated. For doxorubicin experiments, five minutes prior to euthanasia mice were also infused with a 20 mg/kg doxorubicin solution (D-1515.10 mg, in sterile saline, Sigma) over one minute.

While under terminal anesthesia, mice were perfused with 4% paraformaldehyde in PBS, pH 7.4. Perfused tissues were harvested, fixed overnight in 4% paraformaldehyde in PBS, pH 7.4 and transferred to 70% ethanol. Tissues were embedded in paraffin, sectioned, rehydrated, and counterstained with DAPI. The lectin labeling experiments were reproduced independently in a second laboratory (SRH, KI).

To evaluate the influence of lectin administration on doxorubicin distribution, 2 KPC mice where infused with doxorubicin only and processed as described above. No differences in the diffusion of doxorubicin were noted. Conversely, several mice (syngeneic, N=2; KPC, N=1) received lectin only to exclude the possibility that doxorubicin interfered with labeling of endothelial cells by Lycopersicon esculentum lectin. No differences in lectin labeling were noted in this setting.

(xviii) Laser Scanning Cytometry

Mice were perfused with 4% paraformaldehyde in PBS, pH 7.4, while under terminal anesthesia. Perfused tissues were harvested, fixed overnight in 4% paraformaldehyde in PBS, pH 7.4 and transferred to 70% ethanol. Tissues were embedded in paraffin, sectioned, rehydrated, and counterstained with DAPI. Doxorubicin fluorescence was determined by quantitative imaging cytometry using the iCys Research Imaging Cytometer (CompuCyte, Cambridge, Mass.) with iNovator software (CompuCyte).

A scanning protocol for quantification was configured with two channels. Nuclear DAPI fluorescence was excited by the 405 nm diode laser and detected in the blue (445-485 nM) channel and doxorubicin fluorescence was excited by the argon 488 nM laser and detected in the orange (565-595 nM) channel. The threshold in the DAPI channel was optimised to selectively contour individual cells allowing fluorescence measurement within the primary and peripheral nuclear contours.

High resolution tissue scans were acquired from freshly prepared tissue sections using the 63× objective and 0.5 mm step size. Tumour and control areas were defined and doxorubicin fluorescence per cell and cell area measurements were taken from within these regions. Mean fluorescence values and standard deviations for each region were determined as integral fluorescence per cell/cell area.

(xix) Immunofluorescence

Mice were infused with 30 ml of 4% PFA pH 7.4 using a Harvard Apparatus PhD 2000 syringe pump at a rate of 420 ml/min. Tissues were fixed in a 4% PFA pH 7.4 solution for 24 hours and transferred to 70% ethanol. Tissues were paraffin embedded, sectioned and rehydrated. Sections were unmasked in 10 mM citric acid in a microwave for 10 minutes. This unmasking procedure was found to effectively quench the fluorescence of doxorubicin in tissues, allowing the use of additional fluorophores for co-immunofluorescence. Sections were blocked with 10% Serum (D9663, Sigma) in TBST and washed in TBST (Tris Buffered Saline; Tween 20, 1%). The following antibody dilutions were used: CD31, 1:75 (sc-1506, Santa Cruz Biotechnology), AlexaFluor 594, 1:1000 (A11059, Invitrogen). Doxorubicin fluorescence was excited with a 488 nm laser, and emission was detected in a range from 520-620 nm. FIGS. 2B and S2E,F were imaged using a Nikon CC1Si confocal. All other images were acquired on a Leica SP5 confocal microscope.

(xx) Human Tissues

Histological stains and immunohistochemical assessment of CD31 was performed on archival paraffin patient sections from Addenbrooke's Hospital, and patient specimens from the Johns Hopkins Hospital, in accordance with institutional and national policies at the respective locations.

(xxi) RNA Isolation and Quantitative Real-Time PCR Analysis

RNA was isolated from tissues using the RNeasy kit (Qiagen). cDNA was synthesized from 1-2 ug of RNA using the AffinityScript QPCR cDNA Synthesis Kit (Stratagene). cDNA was analyzed by quantitative real-time PCR on a 7900HT Real-Time PCR system using relative quantification (ΔΔct) with the taqman gene expression assays (Applied Biosystems) (Table 7). Experiment was performed on 5 KPC tumors and 7 syngeneic tumors derived from K8484 or K8675 cells.

TABLE 7

| | |
|---|---|
| Actin | Mm00607939_s1 |
| dCK | Mm00432794_m1 |
| ENT1 | Mm00452176_m1 |
| ENT2 | Mm00432817_m1 |
| RRM1 | Mm00432794_m1 |
| RRM2 | Mm00485881_g1 |
| Gli1 | Mm00494645_m1 |
| TK2 | Mm00445175_m1 |

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of:
increasing delivery of an agent to a tissue, or treating or preventing tumor metastasis in a mammal, comprising administering a hedgehog pathway inhibitor and said agent to said tissue;
increasing blood density in a tissue, reducing stromal content in a tissue, increasing blood vessel patency in a tissue, or promoting angiogenesis in a tissue, comprising administering a hedgehog pathway inhibitor to said tissue; or
imaging a tissue, comprising the steps of administering a hedgehog inhibitor to said tissue and using an imaging technique to image said tissue; or
imaging a tissue comprises the steps of administering a hedgehog pathway inhibitor and an imaging agent to said tissue and using an imaging technique to image said tissue.

2. The method of claim 1, wherein said hedgehog pathway inhibitor and said agent are administered concurrently.

3. The method of claim 1, wherein said hedgehog pathway inhibitor and said agent are administered sequentially.

4. The method of claim 1, wherein said agent is a chemotherapeutic agent, a therapeutic agent or an imaging agent.

5. The method of claim 4, wherein said imaging agent is a magnetic resonance imaging (MRI) contrast agent, computerized axial tomography (CAT) contrast agent, or positron emission tomography (PET) contrast agent.

6. (canceled)

7. The method of claim 1, wherein said tissue comprises autochthonous tissue; stromal tissue; ischemic tissue; tumor tissue; tumor tissue exhibiting Hedgehog activation; tumor tissue exhibiting Hedgehog activation characterized by one or more of phenotypes selected from group consisting of a Patched (Ptc) loss-of-function phenotype or a Smoothened (Smo) gain-of-function phenotype; cardiac tissue; or brain tissue.

8-16. (canceled)

17. The method of claim 7, wherein said autochthonous tumor is a pancreatic tumor, a prostate tumor, a breast tumor, a desmoplastic small round cell tumor, a colon tumor, an ovarion tumor, a bladder tumor, or an osteocarcinoma.

18. The method of claim 4, wherein said agent is a chemotherapeutic agent; and wherein said administering comprises administering said hedgehog pathway inhibitor prior to initiating administration of said chemotherapeutic agent; or
said administering comprises administering said hedgehog pathway inhibitor from about 3 days to about 21 days;
said administering comprises administering said hedgehog pathway inhibitor from about 3 days to about 21 days prior to initiating administration of said chemotherapeutic agent; or
said administering comprises administering said hedgehog pathway inhibitor from about 3 days to about 14 days prior to initiating administration of said chemotherapeutic agent.

19-23. (canceled)

24. The method of claim 4, wherein said chemotherapeutic agent is selected from the group consisting of gemcitabine, capecitabine, 5-fluorouracil, floxuridine, doxifluridine, ratitrexed, methotrexate, trimetrexate, thapsigargin, taxol, paclitaxel, docetaxel, actinomycin D, dactinomycin, mercaptopurine, thioguanine, lovastatin, cytosine arabinoside, fludarabine, hydroxyurea, cytarabine, cytarabine, teniposide, topotecan, 9-aminocamptothecin, camptoirinotecan, crisnatol, busulfan, mytomycin C, treosulfan, staurosporine, 1-methyl-4-phenylpyridinium, mercaptopurine, thioguanine, cyclophosphamide, ifosfamide, EB 1089, CB 1093, KH 1060, carmustine, lomustine, mycophenolic acid, tiazofurin, ribavirin, EICAR, cisplatin, carboplatin, oxaliplatin, bevacizumab, mitomycin, dacarbazine, procarbizine, etoposides, prednisolone, trofosfamide, chlorambucil, melphalan, estramustine, dexamethasone, cytarbine, campathecins, bleomycin, doxorubicin, idarubicin, daunorubicin, doxorubicin, epirubicin, pirarubicin, zorubicin, verapamil, mitoxantrone, temozolomide, dactinomycin, plicamycin, bleomycin A2, bleomycin B2, peplomycin, asparaginase, vinblastine, vincristine, vindesine, vinorelbine, imatinib, thalidomide, leucovirin, deferoxamine, lenalidomide, bortezomib, erlotinib, gefitinib, sorafenib, erbitux, and sutinib.

25-33. (canceled)

34. The method of claim 1, wherein said administering comprises administering said hedgehog pathway inhibitor prior to initiating administration of said imaging agent.

35-39. (canceled)

40. The method of claim 1, wherein said imaging technique is ultrasound, X-ray, MRI, CAT, or PET.

41-73. (canceled)

74. The method of claim 1, wherein said hedgehog pathway inhibitor is selected from the group consisting of a compound of Formula I, Formula II, or Formula III: 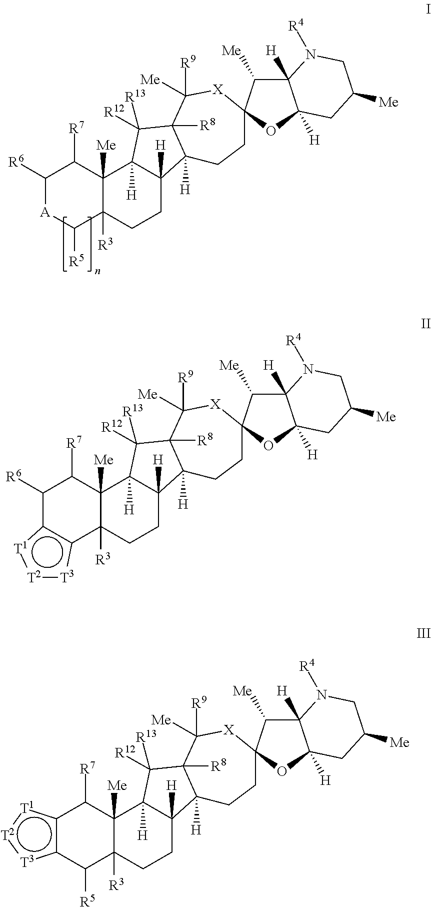
or a pharmaceutically acceptable salt thereof;
wherein A is: 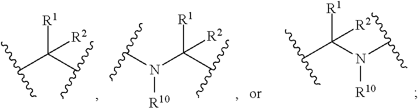
n is 0 or 1;
X is a bond or —CH₂—;
R¹ is selected from —H, optionally substituted alkyl, optionally substituted alkenyl, optionally substituted alkynyl, optionally substituted aryl, optionally substituted aralkyl, optionally substituted cycloalkyl, nitrile, optionally substituted heterocycloalkyl, optionally substituted heteroaryl, —OR¹⁰, —N(R¹⁰)(R¹⁰), —NR¹⁰SO₂R¹⁰, —N(R¹⁰)CO₂R¹⁰, —N(R¹⁰)C(O)R¹⁰, —OC(O)R¹⁰, —C(O)OR¹⁰, —N(R¹⁰)C(O)N(R¹⁰)₂, —N(R¹⁰)S(O)₂N(R¹⁰)₂, and a sugar;
R² is selected from —H, optionally substituted alkyl, optionally substituted alkenyl, optionally substituted alkynyl, optionally substituted aryl, optionally substituted aralkyl, optionally substituted cycloalkyl, nitrile, and optionally substituted heterocycloalkyl; or R¹ and R² taken together form ═O, ═S, ═N(OR), ═N(R)—, ═N(NR₂), ═C(R)₂;
R³ and R⁵, are, independently, selected from —H, optionally substituted alkyl, optionally substituted aralkyl, optionally substituted alkenyl, and optionally substituted alkynyl; or R³ and R⁵ taken together form a bond;
R⁶ and R⁷ are, independently, selected from —H, optionally substituted alkyl, optionally substituted aralkyl, optionally substituted alkenyl, and optionally substituted alkynyl; or R⁶ and R⁷ taken together form a bond;
R⁸ and R⁹ taken together form a bond;
R⁴ is selected from —H, optionally substituted alkyl, optionally substituted alkenyl, optionally substituted alkynyl, optionally substituted aryl, optionally substituted aralkyl, optionally substituted cycloalkyl, optionally substituted heterocycloalkyl, optionally substituted aralkyl, optionally substituted heteroaryl, optionally substituted heteroaralkyl, optionally substituted haloalkyl, —OR¹⁰, —C(O)R¹⁰, —CO₂R¹⁰, —SO₂R¹⁰, —C(O)N(R¹⁰)(R¹⁰), —[C(R)₂]_{q}—R¹⁰, —[(W)—N(R¹⁰)C(O)]_{q}R¹⁰, —[(W)—C(O)]_{q}R¹⁰, —[(W)—C(O)O]_{q}R¹⁰, —[(W)—OC(O)]_{q}R¹⁰, —[(W)—SO₂]_{q}R¹⁰, —[(W)—N(R¹⁰)SO₂]_{q}R¹⁰, —[(W)—C(O)N(R¹⁰)]_{q}R¹⁰, —[(W)—O]_{q}R¹⁰, —[(W)—N(R)]_{q}R¹⁰, and —[(W)—S]_{q}R¹⁰;
each q, independently, for each occurrence, is 1, 2, 3, 4, 5, or 6;
each R¹⁰ is, independently, selected from —H, optionally substituted alkyl, optionally substituted alkenyl, optionally substituted aralkyl, optionally substituted alkynyl, optionally substituted aryl, optionally substituted cycloalkyl, optionally substituted heterocycloalkyl, optionally substituted aralkyl, optionally substituted heteroaryl, optionally substituted heteroaralkyl and —[C(R)₂]ₚ—R¹¹; wherein p is 0-6; or any two occurrences of R¹⁰ on the same substituent can be taken together to form a 4-8 membered optionally substituted ring which contains 0-3 heteroatoms selected from nitrogen, oxygen, sulfur, and phosphorus;
each R¹¹ is, independently, selected from hydroxyl, —N(R)COR, —N(R)C(O)OR, —N(R)SO₂(R), —C(O)N(R)₂, —OC(O)N(R)(R), —SO₂N(R)(R), —N(R)(R), —COOR, —C(O)N(OH)(R), —OS(O)₂OR, —S(O)₂OR, —S(O)₂R, —OP(O)(OR)(OR), —NP(O)(OR)(OR), and —P(O)(OR)(OR);
each R is, independently, selected from —H, optionally substituted alkyl, optionally substituted alkenyl, optionally substituted alkynyl, optionally substituted aryl, optionally substituted cycloalkyl and optionally substituted aralkyl;
R¹² and R¹³ are, independently, selected from —H, optionally substituted alkyl, optionally substituted alkenyl, optionally substituted alkynyl, optionally substituted aryl, optionally substituted aralkyl, optionally substituted cycloalkyl, nitrile, optionally substituted heterocycloalkyl, —OR¹⁰, —N(R¹⁰)(R¹⁰), —NR¹⁰SO₂R¹⁰, —N(R¹⁰)CO₂R¹⁰, —N(R¹⁰)C(O)R¹⁰, and —OC(O)R¹⁰; or R¹² and R¹³ taken together form ═O, ═S, ═N(OR), ═N(R)—, ═N(NR₂), ═C(R)₂;
each W is, independently for each occurrence, selected from an optionally substituted alkyl diradical, optionally substituted alkenyl diradical, optionally substituted alkynyl diradical, optionally substituted aryl diradical, optionally substituted cycloalkyl diradical, optionally substituted heterocycloalkyl diradical, optionally substituted aralkyl diradical, optionally substituted heteroaryl diradical and an optionally substituted heteroaralkyl diradical; and
T¹-T²-T³ is selected from Y-B-A¹, B-Y-A¹, and A¹-B-Y; wherein each of A¹ and B is, independently, selected from nitrogen, sulfur and —C(R¹⁴)₂— and Y is selected from —O—, —S—, and —N(R¹⁵)—;
R¹⁴ is, independently, selected from —H, optionally substituted alkyl, optionally substituted alkenyl, optionally substituted alkynyl, optionally substituted cycloalkyl, optionally substituted aryl, optionally substituted aralkyl, optionally substituted heteroaryl, optionally substituted heteroaralkyl, perhaloalkyl, halo, nitro, nitrile, —SR¹⁰, —OR¹⁰, —N(R¹⁰)(R¹⁰), —C(O)R¹⁰, —CO₂R¹⁰, —OC(O)R¹⁰, —C(O)N(R¹⁰)(R¹⁰), —N(R¹⁰)C(O)R¹⁰, —N(R¹⁰)C(O)N(R¹⁰)(R¹⁰), —S(O)R¹⁰, —S(O)₂R¹⁰, —S(O)₂N(R¹⁰)(R¹⁰), —N(R¹⁰)S(O)₂R¹⁰ and —[C(R¹⁰)₂]_{q}—R¹¹; or two R¹⁴ groups together form ═O; and
R¹⁵ is selected from —H, optionally substituted alkyl, optionally substituted alkenyl, optionally substituted alkynyl, optionally substituted cycloalkyl, optionally substituted aryl, optionally substituted aralkyl, optionally substituted heteroaryl, optionally substituted heteroaralkyl, perhaloalkyl, —C(O)R¹⁰, —CO₂R¹⁰, —C(O)N(R¹⁰)(R¹⁰), —S(O)R¹⁰, —S(O)₂R¹⁰, —S(O)₂N(R¹⁰)(R¹⁰), and —[C(R)₂]_{q}—R¹¹.

75. The method of claim 74, wherein:
A is: 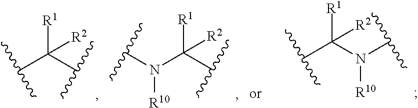
n is 0 or 1;
X is a bond or —CH₂—;
R¹ is selected from —H, optionally substituted alkyl, optionally substituted alkenyl, optionally substituted alkynyl, optionally substituted aryl, optionally substituted aralkyl, optionally substituted cycloalkyl, nitrile, optionally substituted heterocycloalkyl, —OR¹⁰, —N(R¹⁰)(R¹⁰), —NR¹⁰SO₂R¹⁰, —N(R¹⁰)CO₂R¹⁰, —N(R¹⁰)C(O)R¹⁰, —OC(O)R¹⁰, and a sugar;
R² is selected from —H, optionally substituted alkyl, optionally substituted alkenyl, optionally substituted alkynyl, optionally substituted aryl, optionally substituted aralkyl, optionally substituted cycloalkyl, nitrile, and optionally substituted heterocycloalkyl; or R¹ and R² taken together form ═O, ═S, ═N(OR), ═N(R)—, ═N(NR₂), ═C(R)₂;
R³ and R⁵, are, independently, selected from —H, optionally substituted alkyl, optionally substituted aralkyl, optionally substituted alkenyl, and optionally substituted alkynyl; or R³ and R⁵ taken together form a bond;
R⁶ and R⁷ are, independently, selected from —H, optionally substituted alkyl, optionally substituted aralkyl, optionally substituted alkenyl, and optionally substituted alkynyl; or R⁶ and R⁷ taken together form a bond;
R⁸ and R⁹ taken together form a bond;
R⁴ is selected from —H, optionally substituted alkyl, optionally substituted alkenyl, optionally substituted alkynyl, optionally substituted aryl, optionally substituted aralkyl, optionally substituted cycloalkyl, optionally substituted heterocycloalkyl, optionally substituted aralkyl, optionally substituted heteroaryl, optionally substituted heteroaralkyl, optionally substituted haloalkyl, —OR¹⁰, —C(O)R¹⁰, —CO₂R¹⁰, —SO₂R¹⁰, —C(O)N(R¹⁰)(R¹⁰), —[C(R)₂]_{q}—R¹⁰, —[(W)—N(R¹⁰)C(O)]_{q}R¹⁰, —[(W)—C(O)]_{q}R¹⁰, —[(W)—C(O)O]_{q}R¹⁰, —[(W)—OC(O)]_{q}R¹⁰, —[(W)—SO₂]_{q}R¹⁰, —[(W)—N(R¹⁰)SO₂]_{q}R¹⁰, —[(W)—C(O)N(R¹⁰)]_{q}R¹⁰, —[(W)—O]_{q}R¹⁰, —[(W)—N(R)]_{q}R¹⁰, and —[(W)—S]_{q}R¹⁰;
each q, independently, for each occurrence, is 1, 2, 3, 4, 5, or 6;
each R¹⁰ is, independently, selected from —H, optionally substituted alkyl, optionally substituted alkenyl, optionally substituted aralkyl, optionally substituted alkynyl, optionally substituted aryl, optionally substituted cycloalkyl, optionally substituted heterocycloalkyl, optionally substituted aralkyl, optionally substituted heteroaryl, optionally substituted heteroaralkyl and —[C(R)₂]ₚ—R¹¹; wherein p is 0-6; or any two occurrences of R¹⁰ on the same substituent can be taken together to form a 4-8 membered optionally substituted ring which contains 0-3 heteroatoms selected from nitrogen, oxygen, sulfur, and phosphorus;
each R¹¹ is, independently, selected from hydroxyl, —N(R)COR, —N(R)C(O)OR, —N(R)SO₂(R), —C(O)N(R)₂, —OC(O)N(R)(R), —SO₂N(R)(R), —N(R)(R), —COOR, —C(O)N(OH)(R), —OS(O)₂OR, —S(O)₂OR, —S(O)₂R, —OP(O)(OR)(OR), —NP(O)(OR)(OR), and —P(O)(OR)(OR);
each R is, independently, selected from —H, optionally substituted alkyl, optionally substituted alkenyl, optionally substituted alkynyl, optionally substituted aryl, optionally substituted cycloalkyl and optionally substituted aralkyl;
R¹² and R¹³ are, independently, selected from —H, optionally substituted alkyl, optionally substituted alkenyl, optionally substituted alkynyl, optionally substituted aryl, optionally substituted aralkyl, optionally substituted cycloalkyl, nitrile, optionally substituted heterocycloalkyl, —OR¹⁰, —N(R¹⁰)(R¹⁰), —NR¹⁰SO₂R¹⁰, —N(R¹⁰)CO₂R¹⁰, —N(R¹⁰)C(O)R¹⁰, and —OC(O)R¹⁰; or R¹² and R¹³ taken together form ═O, ═S, ═N(OR), ═N(R)—, ═N(NR₂), ═C(R)₂;
each W is, independently for each occurrence, selected from an optionally substituted alkyl diradical, optionally substituted alkenyl diradical, optionally substituted alkynyl diradical, optionally substituted aryl diradical, optionally substituted cycloalkyl diradical, optionally substituted heterocycloalkyl diradical, optionally substituted aralkyl diradical, optionally substituted heteroaryl diradical and an optionally substituted heteroaralkyl diradical; and
T¹-T²-T³ is selected from Y-B-A¹, B-Y-A¹, and A¹-B-Y; wherein each of A¹ and B is, independently, selected from nitrogen, sulfur and —C(R¹⁴)₂— and Y is selected from —O—, —S—, and —N(R¹⁵)—;
wherein R¹⁴ is, independently, selected from —H, optionally substituted alkyl, optionally substituted alkenyl, optionally substituted alkynyl, optionally substituted cycloalkyl, optionally substituted aryl, optionally substituted aralkyl, optionally substituted heteroaryl, optionally substituted heteroaralkyl, perhaloalkyl, halo, nitro, nitrile, —SR¹⁰, —OR¹⁰, —N(R¹⁰)(R¹⁰), —C(O)R¹⁰, —CO₂R¹⁰, —OC(O)R¹⁰, —C(O)N(R¹⁰)(R¹⁰), —N(R¹⁰)C(O)R¹⁰, —N(R¹⁰)C(O)N(R¹⁰)(R¹⁰), —S(O)R¹⁰, —S(O)₂R¹⁰, —S(O)₂N(R¹⁰)(R¹⁰), —N(R¹⁰)S(O)₂R¹⁰ and —[C(R¹⁰)₂]_{q}—R¹¹; and wherein R¹⁵ is selected from —H, optionally substituted alkyl, optionally substituted alkenyl, optionally substituted alkynyl, optionally substituted cycloalkyl, optionally substituted aryl, optionally substituted aralkyl, optionally substituted heteroaryl, optionally substituted heteroaralkyl, perhaloalkyl, —C(O)R¹⁰, —CO₂R¹⁰, —C(O)N(R¹⁰)(R¹⁰), S(O)R¹⁰, —S(O)₂R¹⁰, —S(O)₂N(R¹⁰)(R¹⁰), and —[C(R)₂]_{q}—R¹¹; or two R¹⁴ groups together form ═O; and
R¹⁵ is selected from —H, optionally substituted alkyl, optionally substituted alkenyl, optionally substituted alkynyl, optionally substituted cycloalkyl, optionally substituted aryl, optionally substituted aralkyl, optionally substituted heteroaryl, optionally substituted heteroaralkyl, perhaloalkyl, —C(O)R¹⁰, —CO₂R¹⁰, —C(O)N(R¹⁰)(R¹⁰), S(O)R¹⁰, —S(O)₂R¹⁰, —S(O)₂N(R¹⁰)(R¹⁰), and —[C(R)₂]_{q}—R¹¹.

76. The method of claim 74, wherein the compound is a compound of formula I-32: 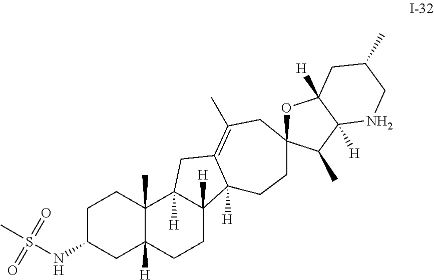
or a pharmaceutically acceptable salt thereof.

77. The method of claim 76, wherein the compound is

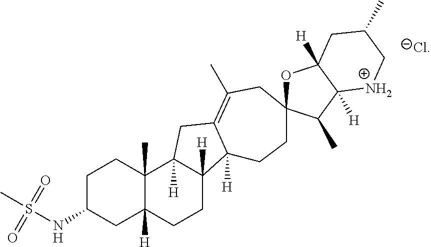

78. The method of claim 1, wherein said hedgehog pathway inhibitor is selected from MK-4101, GDC-0449, BMS-833923, LDE-225, PF-04449913, robotnikinin, and Cur-61414.

79. The method of claim 1, wherein said hedgehog pathway inhibitor is MK-4101

80-106. (canceled)
